SEQUENCE LISTING (19 sequences)

SEQ ID NO: 1 - protein, 7 aa, synthetic construct
PKKKRKV

SEQ ID NO: 2 - protein, 10 aa, synthetic construct
PKKKRKVEAS

SEQ ID NO: 3 - protein, 16 aa, synthetic construct
KRPAATKKAGQAKKKK

SEQ ID NO: 4 - protein, 9 aa, synthetic construct
PAAKRVKLD

SEQ ID NO: 5 - protein, 11 aa, synthetic construct
RQRRNELKRSP

SEQ ID NO: 6 - protein, 38 aa, synthetic construct
NQSSNFGPMKGGNFGGRSSGPYGGGGQYFAKPRNQGGY

SEQ ID NO: 7 - protein, 42 aa, synthetic construct
RMRIZFKNKGKDTAELRRRRVEVSVELRKAKKDEQILKRRNV

SEQ ID NO: 8 - protein, 8 aa, synthetic construct
VSRKRPRP

SEQ ID NO: 9 - protein, 8 aa, synthetic construct
PPKKARED

SEQ ID NO: 10 - protein, 8 aa, synthetic construct
PQPKKKPL

SEQ ID NO: 11 - protein, 12 aa, synthetic construct
SALIKKKKKMAP

SEQ ID NO: 12 - protein, 5 aa, synthetic construct
DRLRR

SEQ ID NO: 13 - protein, 7 aa, synthetic construct
PKQKKRK

SEQ ID NO: 14 - protein, 10 aa, synthetic construct
RKLKKKIKKL

SEQ ID NO: 15 - protein, 10 aa, synthetic construct
REKKKFLKRR

SEQ ID NO: 16 - protein, 20 aa, synthetic construct
KRKGDEVDGVDEVAKKKSKK

SEQ ID NO: 17 - protein, 17 aa, synthetic construct
RKCLQAGMNLEARKTKK

SEQ ID NO: 18 - protein, 288 aa, synthetic construct
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 19 - protein, 183 aa, synthetic construct
RPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRAS

(19) United States
(12) Patent Application Publication
(10) Pub. No.: US 2026/0125683 A1
(43) Pub. Date: May 7, 2026

(54) TREATMENT OF CANCERS HAVING A DRUG-RESISTANT MESENCHYMAL CELL STATE

(71) Applicant: DANA-FARBER CANCER INSTITUTE, INC., Boston, MA (US)

(72) Inventors: Andrew AGUIRRE, Boston, MA (US); Kevin KAPNER, Boston, MA (US); Kyle EVANS, Boston, MA (US)

(73) Assignee: DANA-FARBER CANCER INSTITUTE, INC., Boston, MA (US)

(21) Appl. No.: 19/327,780

(22) Filed: Sep. 12, 2025

Related U.S. Application Data
(63) Continuation of application No. PCT/US2024/019759, filed on Mar. 13, 2024.
(60) Provisional application No. 63/451,800, filed on Mar. 13, 2023.

(51) Int. Cl. C12N 15/113; A61K 48/00; C12N 9/22; C12N 15/11; C12Q 1/6886

(52) U.S. Cl. CPC C12N 15/1137; A61K 48/005; C12N 9/222; C12N 15/111; C12Q 1/6886; C07K 2319/80; C12N 2310/11; C12N 2310/14; C12N 2310/20; C12Q 2600/106; C12Q 2600/158; C12Y 207/07041

(57) ABSTRACT

The subject matter disclosed herein is generally directed to methods of treating a tumor capable of an epithelial-to-mesenchymal transition (an EMT cancer) by targeting the mesenchymal cell state. Disclosed are novel gene dependencies in the mesenchymal cell state. Also, disclosed are novel drugs that target the mesenchymal cell state.

### CROSS-REFERENCE TO RELATED APPLICATION

The present application is a continuation application of International Application No. PCT/US2024/019759, filed on Mar. 13, 2024, which is related to and claims priority under 35 U.S.C. § 119(e) to U.S. provisional patent application No. 63/451,800, entitled “Treatment of Cancers having a Drug-Resistant Mesenchymal Cell State,” filed Mar. 13, 2023. The entire content of the aforementioned patent applications is incorporated herein by this reference.

### SEQUENCE LISTING

The instant application contains a Sequence Listing which has been filed electronically in XML format and is hereby incorporated by reference in its entirety. Said XML copy, created on Sep. 12, 2025, is named “BI11045_SL.xml” and is 18,200 bytes in size.

### TECHNICAL FIELD

The subject matter disclosed herein is generally directed to methods of treating cancers having a drug-resistant mesenchymal cell state by targeting novel vulnerabilities in tumor cells expressing a mesenchymal signature.

### BACKGROUND

Epithelial-to-mesenchymal transition (EMT) occurs in cancer when a well differentiated, epithelial cancer cell undergoes a transcriptional rewiring and evolves into a more mesenchymal, poorly differentiated state. EMT in cancer has been associated with enhanced capacity for invasion as well as drug resistance to both targeted therapy agents as well as standard chemotherapy regimens. Recent single-cell RNA sequencing studies have led to an increased appreciation for the heterogeneous existence of mesenchymal cell states within adenocarcinomas at baseline, before treatment. Moreover, in the context of chemotherapy treatment, these mesenchymal cell states become enriched in the drug-resistant population of cells left after completion of therapy. Moreover, profound plasticity and EMT is postulated to occur in the context of therapy, leading to drug-resistant mesenchymal cell states. Identifying novel strategies to target these mesenchymal cell states may have profound implications for cancer therapy.

Citation or identification of any document in this application is not an admission that such a document is available as prior art to the present disclosure.

### SUMMARY

In one aspect, the instant disclosure provides a method for treating a subject having a cancer capable of an epithelial-to-mesenchymal transition (an EMT cancer), the method involving administering to the subject YM-155 or a derivative thereof, thereby treating the subject having the EMT cancer.

Another aspect of the disclosure provides a method for treating a subject having a cancer capable of an epithelial-to-mesenchymal transition (an EMT cancer), the method involving administering to the subject niclosamide or a derivative thereof, thereby treating the subject having the EMT cancer.

An additional aspect of the disclosure provides a method for treating a subject having a cancer capable of an epithelial-to-mesenchymal transition (an EMT cancer), the method involving administering to the subject one or more of bis.maltolato.oxovanadium.IV, digoxin, GS.9973, LE.135, thiram, butamben, talazoparib, cycloheximide, X2.3.DCPE, epirubicin, etoposide.phosphate, verubulin, indisulam, tasisulam, vinflunine, vindesine, dinaciclib, streptozotocin, Olaparib, PHA.680632, entinostat, SB.225002, oligomycin.A, ouabain, STF.31, lovastatin, LY2183240, BI.2536, SCH.79797, vincristine, homoharringtonine, tipifarnib, tivantinib, methotrexate, axitinib, BRD.K97651142, gossypol, and/or phloretin, thereby treating the subject having the EMT cancer.

Another aspect of the disclosure provides a method for treating a subject having a cancer capable of an epithelial-to-mesenchymal transition (an EMT cancer), the method involving administering to the subject one or more agents capable of inhibiting CDS2 expression or activity, thereby treating the subject having the EMT cancer.

An aspect of the disclosure provides a method for treating a subject having a cancer capable of an epithelial-to-mesenchymal transition (an EMT cancer), the method involving administering to the subject one or more agents capable of inhibiting the expression or activity of CDS2, ELMO2, ITGAV, AP1M1, FERMT2, CHMP4B, PTK2, ITGB5, PRKAR1A, ACTR1A, SRF, ZEB1, MTOR, DNM1L PSMB7, MOB4, TEAD1, DOCK5, ACTR3, BCAR1, UFM1, TIPARP, ILK, RSU1, WWTR1, NCKAP1, ATP6V1C1, MYH9, FOSL1, LIMS1, GNB1, EFR3A, ARHGEF7, WDR7, PKN2, CEMIP2, TEAD1, CTDNEP1, MARK3, VCL, CDC42, BCAR1, EGLN1, UFM1, GPX4, and/or CRK, thereby treating the subject having the EMT cancer.

In some embodiments, the method further includes identifying the cancer as an EMT cancer by a step involving detecting in the cancer the expression of one or more genes shown in any of FIGS. 3A-3C as positively correlated with EMT Down signatures (e.g., PTPN6, ILDR1, TSTD1, LCN2, MYO6, MFSD6, AMN, DDR1, PPL, C2orf15, AREG, CD9, FAM160A1, ENPP5, CD24, SMIM22, RAB25, PRRG2, CLDN4, RNF223, PLA2G4F, LLMO3, MAP7, MAL2, CBLC, KRTCAP3, MACC1, F11R, SPINT1, CNKSR1, CGN, IRF6, ESRP2, MPZL3, OVOL2, BSPRY, PRSS22, FXYD3, TJP2, PKP2, TMC5, RNF43, USP43, DLG3, PRKCZ, PKP3, ANO9, GRB7, JUP, CDH3, C11orf52, MYO5B, PATJ, TSPAN1, ZNF165, EPHA1, ELF3, BICDL22, TJP3, MUC20, MARVELD2, TC2N, CDS1, CLDN7, GRHL2, C1orf116, EPN3, PSD4, FAAH, OVOL1, SYNE4, IQANK1, MARVELD3, CRB3, KDF1, PRSS8, MAPK3, MAPK13, S100A14, ERBB3, GALNS, KRT19, EPS8L1, TMC4, ESRP1, SOWAHB, ST14, CDC42BPG, RAB17, CDH1, SH2D3A, LSR, B3GN3T, C1orf210, MPZL2, FUT3, SPINT2, C6orf132, PLEKHA7, TMC5, ICA1, TPD52L1, KLK8, SFTA2, KRT8, LAD1, IRF6, KLF5, PRR15, BICDL2, LIPH, LSR, FXYD3, EPCAM, MAL2, C2N, SPINT1, TCM5, ASS1, COBL, ACSL5, GPX2, MYZAP, KIAA1217, SCNN1A, B3GNT3, MISP, VGLL1, ITGB6, DOK7, HSH2D, SYT8, SH3YL1, SFN, TMEM125, TTC9, CST6, ACOT11, TUBA4A, PERP, ABHD11, BTC, CJB3, TNK1, LRRC1, ARRDC1, ANXA3, C11orf52, TCM4, ADIRF, LPAR5, GJB4, GJB5, MCTP2, TRIM31, HID1, DAPP1, SLPI, MST1R, LY75, CRYBG2, FUCA1, EPHA1, BIK, C1orf210, JUP, DDR1, LRG1, VAMP8, F3. CD9, TNK1, ILDR1, ITGB4, ESRP1, GALNT3, RNF223 MPZL2, EPCAM, B3GNT3, ST14, PTK7, GPR87, KCNK6, UPK2, HSH2D, KLF5, TMPRSS4, FRK, FAM83F, KRT19, TMC4, TC2N, PRRG2, RBM47, TC22, ITGB6, and/or TMEM125) and/or as negatively correlated with EMT Up signatures (e.g., PTCH2, TET3, AUNIP, E2F2, BEND3, CCSAP, INPP5J, RMND5B, PHF8, RAB11FIP4, OTUD3, KCNJ11, ZBTB39, STRBP, FZD3, SNK1, STRBP, PPFIA3, ADGRL1, FEM1A, MYCL, CHTOP, TICRR, CDS1, LHX4, PCNT, TCLD2, HDHD3, STRBP, CAMSAP3, C2orf15, DENND1C, IQANK1, MARVELD3, ZDHHC23, LNX2, ESRP2, BSPRY, LLGL2, SKY, CRB3, MAP7, MYH14, ERBB3, KDF1, OVOL1, HSDIIB2, GLS2, MARVELD2, ARHGAP8, PRR5, CDS1, APlAr, EPB41, TET3, ESRP2, GAN, CDC42BPG, POU2F1, DDI2, ARHGAP8, STRBP, RREB1, LRRC8B, LLGL2, GRTP1, HOOK1, BSPRY, RDH13, SPATA2L, MCM9, PRR5, and/or RDH13).

In certain embodiments, the method further includes identifying the cancer as an EMT cancer by a step involving detecting in the cancer or subject the presence of one or more of the following metabolites: stearoylcarnitine, C16.0.SM, betaine, palmitoylcarnitine, adenosine, 6.phosphogluconate, 1.methylnicotinamide, anthranilic.acid, carnosine, and/or sorbitol.

Another aspect of the disclosure provides a method for identifying and treating a subject having a cancer capable of an epithelial-to-mesenchymal transition (an EMT cancer), the method involving: (a) detecting the expression in the cancer of one or more genes identified in FIG. 5B as positively correlated with CDS2 dependency; and (b) administering to the subject one or more agents capable of inhibiting CDS2 expression or activity, thereby identifying and treating the subject having the EMT cancer.

In one embodiment, the one or more genes identified in FIG. 5B as positively correlated with CDS2 dependency (e.g., PKP3, CDS1, MAL2, ELF3, IRF6, PATJ, CDH1, MAP7, ARHGEF16, ARHGEF5, TNK1, CNKSR1, ARHGAP8, CLDN4, IQANK1, CDH3, OVOL1, C11orf52, GRB7, GRHL2, C6orf132, MARVELD3, ESRP1, MPZL2, CHMP4C, TMEM125, TACSTD2, MYO5B, PRSS22, S100A14, SRP2, CRB3, EPCAM, RAB25, CLBC, TMEM184A, CLDN7, PRSS8, PRRG2, and/or SFN) is detected in the cancer below a reference value. Optionally, the reference value is determined by comparison to an appropriate control sample lacking the EMT cancer.

In another embodiment, the one or more genes include CDS1.

A further aspect of the disclosure provides a method for identifying and treating a subject having a cancer capable of an epithelial-to-mesenchymal transition (an EMT cancer), the method involving: (a) detecting in the subject one or more metabolites identified in FIG. 6A as negatively correlated with CDS2 dependency; and (b) administering to the subject one or more agents capable of inhibiting CDS2 expression or activity, thereby identifying and treating the subject having the EMT cancer.

In one embodiment, the one or more metabolites identified in FIG. 6A as negatively correlated with CDS2 dependency (e.g., oxalate, C38.4.PC, AMP, C40.6.PC, C56.5.TAG, C50.0.TAG, trimethylamine.N.oxide, C58.7.TAG, C56.7.TAG, C36.4.PC.A, UMP, C22.1.SM, dCMP, C56.6.TAG, C58.6.TAG, C36.4.PC.B, C58.8.TAG, cytidine, CMP, C38.6.PC, alpha.glycophosphate, arachidonyl_carnitine, C38.5.PC, and/or C58.8.TAG) is detected in the subject above a reference value. Optionally, the reference value is determined by comparison to an appropriate control subject or sample lacking the EMT cancer.

In another embodiment, the administering step is performed prior to, concurrently with, or after a primary cancer treatment (e.g., a standard of care cancer treatment as described herein and/or as known in the art). Optionally, the primary cancer treatment is a targeted therapy and/or a chemotherapy.

In one embodiment, the method further involves detecting cancer cells that express a mesenchymal signature after the administering step and comparing to the number of cancer cells expressing a mesenchymal signature before the administering step. Optionally, the treatment is identified as efficacious in the subject if the number of tumor cells expressing a mesenchymal signature decreases.

In one embodiment, the one or more agents are one or more small molecules that reduce(s) the activity or expression of CDS2 or the one or more genes.

In a related embodiment, the one or more small molecule(s) is capable of binding to the active site of CDS2, such as, an antagonistic analog of 1-stearoyl-2-arachidonoyl-sn-phosphatidic acid; or an anionic phospholipid end product or analog thereof, such as a 1-stearoyl-2-arachidonoyl species; or phosphatidylinositol-(4,5)-bisphosphate or derivatives thereof.

In certain embodiments, the one or more small molecules is a small molecule degrader capable of degrading CDS2 or the one or more genes. In certain embodiments, the one or more agents is an RNAi or antisense oligonucleotide (ASO). In certain embodiments, the one or more agents is a transcriptional repressor system comprising a DNA binding element linked to or otherwise capable of complexing with a transcriptional repressor and configured to bind an enhancer of CDS2 or the one or more genes. In certain embodiments, the one or more agents is an epigenetic modification polypeptide comprising a DNA binding element linked to or otherwise capable of associating with an epigenetic modification domain such that binding of the DNA binding element at target sequence on gDNA results in one or more epigenetic modifications by the epigenetic modification domain that decreases expression of CDS2 or the one or more genes. In certain embodiments, the DNA binding element comprises a zinc finger protein or DNA-binding domain thereof, TALE protein or DNA-binding domain thereof, or a Cas nuclease protein or DNA-binding domain thereof. In certain embodiments, the one or more agents is a gene editing system configured to modify CDS2 or the one or more genes, an enhancer associated with CDS2 or the one or more genes, or a mRNA encoding CDS2 or the one or more genes, such that expression or activity of CDS2 or the one or more genes is reduced (e.g., by 10% or more, by 20% or more, by 30% or more, etc.). In certain embodiments, the gene editing system is a zinc finger nuclease, a TALEN, a meganuclease, or a CRISPR-Cas system. In certain embodiments, the gene editing system comprises a Cas polypeptide, a guide molecule capable of forming a complex with the Cas polypeptide and directing sequence-specific binding of the complex to a target sequence on the gene or enhancer associated with the gene such that one or more indels or insertions that reduce expression or activity of CDS2 or the one or more genes is introduced into the gene or the enhancer associated with CDS2 or the one or more genes. In certain embodiments, the gene editing system is a base editing system. In certain embodiments, the base editing system is a CRISPR-Cas base editing system. In certain embodiments, the gene editing system is a CRISPR prime editing system. In certain embodiments, the gene editing is a CRISPR-associated transposase (CAST) system. In certain embodiments, the gene editing system comprises an epigenetic modification polypeptide comprising a DNA binding domain linked to or otherwise capable of associating with an epigenetic modification domain such that binding of the DNA binding domain at target sequence on gDNA results in one or more epigenetic modifications by the epigenetic modification domain that decreases expression of the one or more polypeptides. In certain embodiments, the transcriptional repressor system, epigenetic modification polypeptide, or the gene editing system is encoded in a polynucleotide vector. In certain embodiments, the vector is a viral vector.

In certain embodiments, the epithelial-to-mesenchymal transition (EMT) cancer is selected from among: breast cancer, colon cancer, lung cancer, prostate cancer, testicular cancer, brain cancer, skin cancer, rectal cancer, gastric cancer, esophageal cancer, tracheal cancer, head and neck cancer, pancreatic cancer, liver cancer, ovarian cancer, cervical cancer, uterine carcinoma, vulvar cancer, mesothelioma, renal cancer, bladder cancer, thyroid cancer, bone cancer, and/or adrenocortical carcinoma.

A further aspect of the disclosure provides a method for detecting and treating a cancer capable of an epithelial-to-mesenchymal transition (an EMT cancer) in a subject, the method involving: (a) detecting one or more of: (i) expression in the cancer of one or more genes shown in any of FIGS. 3A-3C as positively correlated with EMT Down signatures and/or as negatively correlated with EMT Up signatures in a cancer of the subject; (ii) the presence in the subject of one or more of the following metabolites: stearoylcarnitine, C16.0.SM, betaine, palmitoylcarnitine, adenosine, 6.phosphogluconate, 1.methylnicotinamide, anthranilic.acid, carnosine, and/or sorbitol; (iii) expression in the cancer of one or more genes identified in FIG. 5B as positively correlated with CDS2 dependency; and/or (iv) the presence in the subject of one or more metabolites identified in FIG. 6A as negatively correlated with CDS2 dependency, thereby identifying the cancer as an EMT cancer; and (b) administering to the subject one or more of: niclosamide or a derivative thereof; YM-155 or a derivative thereof, bis.maltolato.oxovanadium.IV; digoxin; GS.9973; LE.135; thiram; butamben; talazoparib; cycloheximide; X2.3.DCPE; epirubicin; etoposide.phosphate; verubulin; indisulam; tasisulam; vinflunine; vindesine; dinaciclib; streptozotocin; Olaparib; PHA.680632; entinostat; SB.225002; oligomycin.A; ouabain; STF.31; lovastatin; LY2183240; BI.2536; SCH.79797; vincristine; homoharringtonine; tipifarnib; tivantinib; methotrexate; axitinib; BRD.K97651142; gossypol; phloretin; one or more agents capable of inhibiting CDS2 expression or activity; and/or one or more agents capable of inhibiting the expression or activity of ELMO2, ITGAV, AP1M1, FERMT2, CHMP4B, PTK2, ITGB5, PRKAR1A, ACTR1A, SRF, ZEB1, MTOR, DNM1L PSMB7, MOB4, TEAD1, DOCK5, ACTR3, BCAR1, UFM1, TIPARP, ILK, RSU1, WWTR1, NCKAP1, ATP6V1C1, MYH9, FOSL1, LIMS1, GNB1, EFR3A, ARHGEF7, WDR7, PKN2, CEMIP2, TEAD1, CTDNEP1, MARK3, VCL, CDC42, BCAR1, EGLN1, UFM1, GPX4, and/or CRK, thereby detecting and treating the EMT cancer in the subject.

Another aspect of the disclosure provides a pharmaceutical composition for treating an EMT cancer in a subject, the pharmaceutical composition including a therapeutically effective amount of one or more of: niclosamide or a derivative thereof; YM-155 or a derivative thereof; bis.maltolato.oxovanadium.IV; digoxin; GS.9973; LE.135; thiram; butamben; talazoparib; cycloheximide; X2.3.DCPE; epirubicin; etoposide.phosphate; verubulin; indisulam; tasisulam; vinflunine; vindesine; dinaciclib; streptozotocin; Olaparib; PHA.680632; entinostat; SB.225002; oligomycin.A; ouabain; STF.31; lovastatin; LY2183240; BI.2536; SCH.79797; vincristine; homoharringtonine; tipifarnib; tivantinib; methotrexate; axitinib; BRD.K97651142; gossypol; phloretin; one or more agents capable of inhibiting CDS2 expression or activity; and/or one or more agents capable of inhibiting the expression or activity of ELMO2, ITGAV, AP1M1, FERMT2, CHMP4B, PTK2, ITGB5, PRKAR1A, ACTR1A, SRF, ZEB1, MTOR, DNM1L PSMB7, MOB4, TEAD1, DOCK5, ACTR3, BCAR1, UFM1, TIPARP, ILK, RSU1, WWTR1, NCKAP1, ATP6V1C1, MYH9, FOSL1, LIMS1, GNB1, EFR3A, ARHGEF7, WDR7, PKN2, CEMIP2, TEAD1, CTDNEP1, MARK3, VCL, CDC42, BCAR1, EGLN1, UFM1, GPX4, and/or CRK, and a pharmaceutically acceptable carrier.

An additional aspect of the disclosure provides a use of one or more agent in the preparation of a medicament for treating an EMT cancer (a cancer capable of an epithelial-to-mesenchymal transition), where the one or more agent is selected from among: niclosamide or a derivative thereof; YM-155 or a derivative thereof, bis.maltolato.oxovanadium.IV; digoxin; GS.9973; LE.135; thiram; butamben; talazoparib; cycloheximide; X2.3.DCPE; epirubicin; etoposide.phosphate; verubulin; indisulam; tasisulam; vinflunine; vindesine; dinaciclib; streptozotocin; Olaparib; PHA.680632; entinostat; SB.225002; oligomycin.A; ouabain; STF.31; lovastatin; LY2183240; BI.2536; SCH.79797; vincristine; homoharringtonine; tipifarnib; tivantinib; methotrexate; axitinib; BRD.K97651142; gossypol; phloretin; one or more agents capable of inhibiting CDS2 expression or activity; and/or one or more agents capable of inhibiting the expression or activity of ELMO2, ITGAV, AP1M1, FERMT2, CHMP4B, PTK2, ITGB5, PRKAR1A, ACTR1A, SRF, ZEB1, MTOR, DNM1L PSMB7, MOB4, TEAD1, DOCK5, ACTR3, BCAR1, UFM1, TIPARP, ILK, RSU1, WWTR1, NCKAP1, ATP6V1C1, MYH9, FOSL1, LIMS1, GNB1, EFR3A, ARHGEF7, WDR7, PKN2, CEMIP2, TEAD1, CTDNEP1, MARK3, VCL, CDC42, BCAR1, EGLN1, UFM1, GPX4, and/or CRK.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present disclosure will be obtained by reference to the following detailed description that sets forth illustrative embodiments, in which the principles of the disclosure may be utilized, and the accompanying drawings of which:

FIG. 1A-1B—Investigation of the correlation of all dependencies with each of the respective EMT signatures. FIG. 1A. Investigation of EMT Down signatures (more epithelial) highlight CDS2 dependency as the top positive correlation, indicating reduced dependency in epithelial cell lines. FIG. 1B. Investigation of EMT Up signatures (more mesenchymal) highlight CDS2 dependency as a negative correlation, indicating increased dependency in mesenchymal cell lines. Dependency scores are normalized such that nonessential genes have a median score of 0 and independently identified common essential genes have a median score of −1, so a stronger dependency is indicated by a more negative dependency score. Positive correlations with dependency scores imply reduced dependency with increasing score and negative correlations imply increased dependency with decreasing score.

FIG. 2A-2B—Investigation of the EMT signature and drug sensitivity data. FIG. 2A illustrates the top and bottom correlations between the PRISM compound AUCs (measure of drug sensitivity with a higher value indicating resistance) across three independent EMT down (FIG. 2A) and up (FIG. 2A continued) signatures (Byers, Groger, and Taube). FIG. 2B illustrates the top and bottom correlations between the CTD² drug testing cohort compound AUCs with the down (FIG. 2B) and up (FIG. 2B continued) EMT signatures (Byers, Groger, and Taube).

FIG. 3A-3D—Investigation of EMT signatures and RNA expression (FIG. 3A-3C) and metabolite levels (FIG. 3D). FIG. 3A-3C shows the Pearson correlation of the RNA expression levels for each gene and the labeled three independent EMT down and up signatures (Byers (FIG. 3A), Groger (FIG. 3B), and Taube (FIG. 3C)). The top labeled points are the top positive correlations, and the bottom labeled points are the top negative correlations. Positive correlations indicate increased RNA expression for that gene with an increasing EMT score (i.e., down or up EMT score), while negative correlations indicate decreased RNA expression for that gene with an increasing EMT score. FIG. 3D shows volcano plots highlighting the top and bottom metabolite Pearson correlations for each of the three independent EMT down and up signatures (Byers, Taube, and Groger) Positive correlations indicate a higher metabolite concentration with an increasing EMT score, while negative correlations indicate a lower metabolite concentration with an increasing EMT score.

FIG. 4A-4B—CDS2 Depmap CRISPR Data. FIG. 4A shows a strong selective dependency with a low expression of CDS1 (paralog relationship). FIG. 4B shows CDS2 dependency score is correlated with CDS1 RNA expression.

FIG. 5A-5C—Investigation of CDS2 dependency with all dependencies. (FIG. 5A) and RNA expression (FIG. 5B). RNA expression is measured in log₂(TPM+1) values. FIG. 5A highlights the co-dependencies (lighter text) with CDS2 and anti-correlated dependencies (darker text) with CDS2. FIG. 5B highlights genes for which high/low RNA expression is correlated with decreased/increased dependency (lighter text) respectively. CDS1 rises as the most significantly correlated gene with CDS2 dependency, indicating that low CDS1 expression is associated with increased CDS2 dependency (and vice versa). FIG. 5C illustrates that the relationship between CDS1 expression and CDS2 dependency is unidirectional. CDS2 dependency and CDS1 expression are correlated, however CDS1 dependency and CDS2 expression are not correlated.

FIG. 6A-6C—Investigation of CDS2 dependency and metabolites. FIG. 6A. dependency, where negative correlations imply an increase in CDS2 dependency results is associated with an increase in metabolite levels. In other words, cells having a greater CDS2 dependency have an increase in the negatively correlated metabolic levels. FIGS. 6B and 6C. Correlation of CDS2 RNA expression and the Cancer Cell Line Encyclopedia (CCLE) metabolite levels was determined, where positive correlations imply an increase in CDS2 expression is associated with an increase in metabolite levels. The top 50 positively correlated metabolites (FIG. 6B) and the top 50 negatively correlated metabolites (FIG. 6C) from FIG. 6A were analyzed with Metaboanalyst software which showed the pathway enrichment (Top 25 gene sets). Freely available example dataset is available for download with no restrictions on usage (www.metaboanalyst.ca).

FIG. 7—EMT proposed targeting. illustrates targeting the mesenchymal state by inhibiting novel targets.

FIG. 8 shows that YM-155 selectively targeted cancer cells that have undergone an epithelial-to-mesenchymal transition (the KP4, Panc1, Miapaca2 and 8988T cancer cell lines indicated as mesenchymal), as compared with more epithelial cancer cells (the Panc0403, Panc0504 and 8988S cell lines indicated as epithelial).

FIG. 9 shows CRISPR-Cas validation of CDS2 dependency in epithelial cancer cell lines (Panc0403, HupT4, YAPC, at left and labeled as “epithelial”) and in mesenchymal cancer cell lines (MIAPACA2, PATU8988T, KP4, at right and labeled as “mesenchymal”), with mesenchymal cancer cell lines demonstrated to exhibit greater CDS2 dependency.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995)(M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar as such variations are appropriate to perform in the aspects and embodiments disclosed herein. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present disclosure encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present disclosure. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the disclosure. For example, in the appended claims, any of the claimed embodiments can be used in any combination.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested. As used herein “treating” includes ameliorating, curing, preventing it from becoming worse, slowing the rate of progression, or preventing the disorder from re-occurring (i.e., to prevent a relapse). In certain embodiments, the present disclosure provides for one or more therapeutic agents against combinations of targets identified. Targeting the identified combinations may provide for enhanced or otherwise previously unknown activity in the treatment of disease.

The term “in need of treatment” as used herein refers to a judgment made by a caregiver (e.g. physician, nurse, nurse practitioner, or individual in the case of humans; veterinarian in the case of animals, including non-human animals) that a subject requires or will benefit from treatment. This judgment is made based on a variety of factors that are in the realm of a caregiver's experience, but that include the knowledge that the subject is ill, or will be ill, as the result of a condition that is treatable by the compositions and therapeutic agents described herein.

The term “adjuvant therapy” as used herein refers to any treatment given after primary therapy to increase the chance of long-term disease-free survival. The term “neoadjuvant therapy” as used herein refers to any treatment given before primary therapy. The term “primary therapy” as used herein refers to the main treatment used to reduce or eliminate the cancer.

All publications, published patent documents, and patent applications cited herein are hereby incorporated by reference to the same extent as though each individual publication, published patent document, or patent application was specifically and individually indicated as being incorporated by reference.

### Overview

To identify novel targets for the drug-resistant mesenchymal cell state, Applicants performed custom analyses of diverse genomic, metabolomic, transcriptomic, functional genetic and small molecule screening datasets to identify cell state-specific features and vulnerabilities. Dependency profiling has been described for cancer cell lines (see, e.g., Boehm J S, Golub T R. An ecosystem of cancer cell line factories to support a cancer dependency map. Nat Rev Genet. 2015; 16(7):373-374. doi:10.1038/nrg3967; Yu C, Mannan A M, Yvone G M, et al. High-throughput identification of genotype-specific cancer vulnerabilities in mixtures of barcoded tumor cell lines. Nat Biotechnol. 2016; 34(4):419-423. doi:10.1038/nbt.3460; Tsherniak A, Vazquez F, Montgomery P G, et al. Defining a Cancer Dependency Map. Cell. 2017; 170(3):564-576.e16. doi:10.1016/j.cell.2017.06.010; Meyers R M, Bryan J G, McFarland J M, et al. Computational correction of copy number effect improves specificity of CRISPR-Cas9 essentiality screens in cancer cells. Nat Genet. 2017; 49(12):1779-1784. doi:10.1038/ng.3984; Dempster J M, Pacini C, Pantel S, et al. Agreement between two large pan-cancer CRISPR-Cas9 gene dependency data sets. Nat Commun. 2019; 10(1):5817. Published 2019 December 20. doi:10.1038/s41467-019-13805-y; Dempster, J. M., Rossen, J., Kazachkova, M., Pan, J., Kugener, G., Root, D. E., & Tsherniak, A. (2019). Extracting Biological Insights from the Project Achilles Genome-Scale CRISPR Screens in Cancer Cell Lines. BioRxiv, 720243; and Dempster J M, Boyle I, Vazquez F, et al. Chronos: a cell population dynamics model of CRISPR experiments that improves inference of gene fitness effects. Genome Biol. 2021; 22(1):343). Applicants have curated RNA signatures from the literature to distinguish epithelial from mesenchymal cell states in cancer dependency profiling data and applied these signatures to define multiple transcriptomic, metabolomic and proteomic features associated with either the epithelial or mesenchymal cell states. Applicants have also identified small molecule inhibitors with selective efficacy in either the epithelial or mesenchymal cell states. Moreover, Applicants have defined selective genetic dependencies representing potential new therapeutic targets for either the epithelial or mesenchymal cell state. In particular, Applicants have identified several high-priority targets.

In one embodiment, the present disclosure provides for a method of treating a tumor capable of an epithelial-to-mesenchymal transition (EMT cancer) in a subject in need thereof by administering to the subject small molecules that target and reduce or eliminate tumor cells having a mesenchymal state. In another embodiment, the present disclosure provides for a method of treating a tumor capable of an epithelial-to-mesenchymal transition (EMT cancer) in a subject in need thereof by administering to the subject one or more agents specific for a gene target that reduce or eliminate tumor cells having a mesenchymal state. Eliminating tumor cells having a mesenchymal state can reduce resistance to primary treatments.

In example embodiments, CDS2 is a mesenchymal-specific drug target. CDS2 is a synthase enzyme that generates precursors of phosphoinositide second messengers for many G-protein-coupled receptors (GPCRs) and tyrosine kinases. CDS1 and CDS2 are highly related, paralog genes. CDS2 is the top-ranked dependency in mesenchymal cells, across multiple, independent genome-wide CRISPR DepMap analyses. The EMT master regulators ZEB1/ZEB2 suppress CDS1 but not CDS2, leading to mesenchymal cell dependency on CDS2 for phosphoinositide generation. Thus, CDS2 is a novel mesenchymal cell state-specific drug target. Previously, there were no known drug development efforts directed at CDS2.

In example embodiments, Niclosamide and YM-155 are mesenchymal-selective small molecule vulnerabilities. Through querying the Broad Institute drug repurposing collection and the CTDD screening collection, Applicants have identified the anti-helminthic agent niclosamide as having selective efficacy in mesenchymal cancer cells. Additionally, Applicants have identified the survivin inhibitor YM-155 as having potent selectivity against the mesenchymal cell state.

These examples represent novel uses of these small molecules in human cancer and could provide a foundation for medicinal chemistry efforts to develop more selective drugs for the mesenchymal cell state.

### Methods of Treating Cancer by Targeting the Mesenchymal Cell State

In one example embodiment, the present disclosure provides for a method of treating a tumor capable of an epithelial-to-mesenchymal transition (EMT cancer) in a subject in need thereof by administering to the subject small molecules that target and reduce or eliminate tumor cells having a mesenchymal state. In another example embodiment, the present disclosure provides for a method of treating a tumor capable of an epithelial-to-mesenchymal transition (EMT cancer) in a subject in need thereof by administering to the subject one or more agents specific for a gene target that reduce or eliminate tumor cells having a mesenchymal state. Eliminating tumor cells having a mesenchymal state can reduce resistance to primary treatments (e.g., standard of care treatments). Applicants discovered that the specific agents and gene targets are specific to vulnerabilities in the mesenchymal tumor cell state, thus allowing Applicants for the first time to provide for novel treatment regimens. For example, the mesenchymal state can be detected before and after a primary treatment and before or after a secondary treatment targeting the mesenchymal cell state as described herein. Furthermore, because resistance is observed after a primary treatment, but may be present before the primary treatment, the agents can be administered before the primary treatment to remove any cells having a mesenchymal cell state. Thus, the treatment targeting a mesenchymal cell state can be targeted before a primary treatment. In an example embodiment, a primary treatment is administered followed by the primary treatment plus the additional agent targeting the mesenchymal cell state. In an example embodiment, the additional agent targeting the mesenchymal cell state is administered followed by the primary treatment. In an example embodiment, the additional agent targeting the mesenchymal cell state is administered followed by the primary treatment plus the additional agent targeting the mesenchymal cell state.

In one example embodiment, the one or more genes targeted are partially exposed on the cell surface, such that they can be targeted with an antibody, adoptive cell transfer, or a small molecule.

### Exemplary Cancers

The present disclosure is useful for the treatment of any cancer that undergoes epithelial-to-mesenchymal transition (EMT), hereafter referred to as an “EMT cancer.” Epithelial-mesenchymal transitions (EMTs), the acquisition of mesenchymal features from epithelial cells, occur during some biological processes and are classified into three types: the first type occurs during embryonic development, the second type is associated with adult tissue regeneration, and the third type occurs in cancer progression (Ribatti D, Tamma R, Annese T. Epithelial-Mesenchymal Transition in Cancer: A Historical Overview. Transl Oncol. 2020 June; 13(6):100773). The classical model of EMT is one in which malignant cells shed their epithelial identity and adopt a mesenchymal phenotype. EMT is characterized by loss of cell polarity, motility, and ability to remodel the extracellular matrix, and ultimately increased invasive potential (Lambert A W, Pattabiraman D R, Weinberg R A. Emerging Biological Principles of Metastasis. Cell. 2017; 168(4):670-91). Studies have suggested that EMT is a process that occurs in all epithelial tumors. In certain embodiments, the methods described herein may be used for any epithelial cancer. EMT signatures have been identified across cancer types (see, e.g., Vasaikar S V, Deshmukh A P, den Hollander P, et al. EMTome: a resource for pan-cancer analysis of epithelial-mesenchymal transition genes and signatures. Br J Cancer. 2021; 124(1):259-269). Cancers that express an EMT signature that can be treated according to the present disclosure may include, but are not limited to, breast cancer, colon cancer (e.g., colorectal carcinoma (CRC), colorectal adenocarcinoma (COAD, READ)), lung cancer, prostate cancer, testicular cancer (e.g., Testicular Germ Cell Tumors (TGCT)), brain cancer (e.g., Glioblastoma multiforme (GBM), Brain Lower Grade Glioma (LGG)), skin cancer (e.g., uveal melanoma (UVM), Skin Cutaneous Melanoma (SKCM), cutaneous squamous cell carcinoma), rectal cancer, gastric cancer (e.g., stomach adenocarcinoma (STAD)), esophageal cancer, tracheal cancer, head and neck cancer (e.g., head and neck squamous cell carcinoma (HNSCC)), pancreatic cancer (e.g., pancreatic adenocarcinoma (PAAD)), liver cancer (e.g., Hepatocellular carcinoma (HCC)), ovarian cancer, cervical cancer (CESC), uterine carcinoma, vulvar cancer, mesothelioma (MESO), renal cancer (e.g., Kidney renal clear cell carcinoma (KIRC), Kidney renal papillary cell carcinoma (KIRP)), bladder cancer (BLCA), thyroid cancer (e.g., papillary thyroid carcinoma (PTC)), bone cancer (e.g., osteosarcoma), adrenocortical carcinoma (ACC), carcinomas, sarcomas, and soft tissue cancers. Thus, the disclosure is generally applicable to any type of cancer in which expression of an EMT program occurs. In certain embodiments, the method of treatment is useful for all epithelial tumors. As used herein, epithelial tumor refers to any cancer that begins in the cells that line an organ, including but not limited to lung cancer, breast cancer, prostate cancer, colon cancer, skin cancer, head and neck cancer, ovarian cancer, cervical cancer, uterine carcinoma, and esophageal carcinoma.

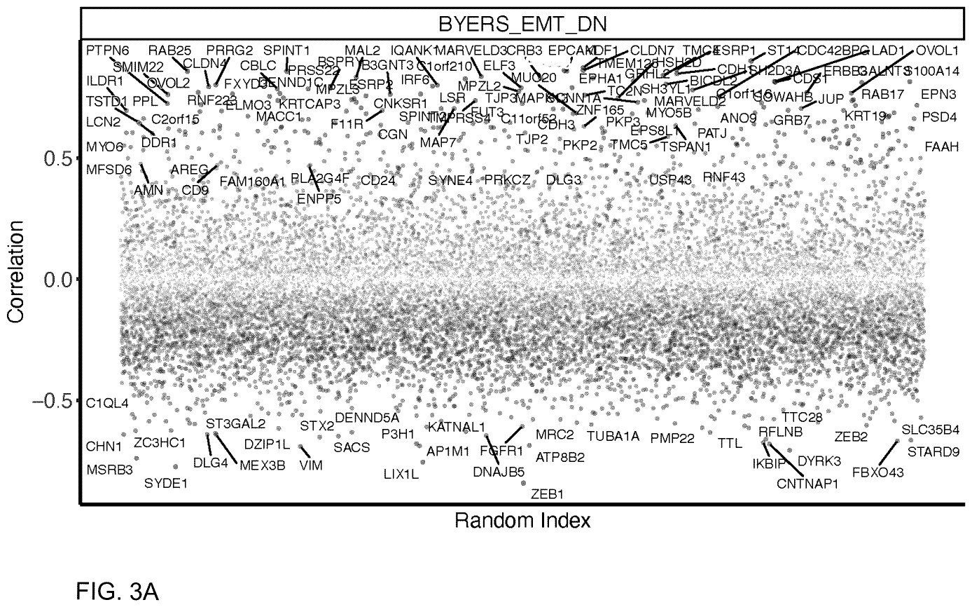

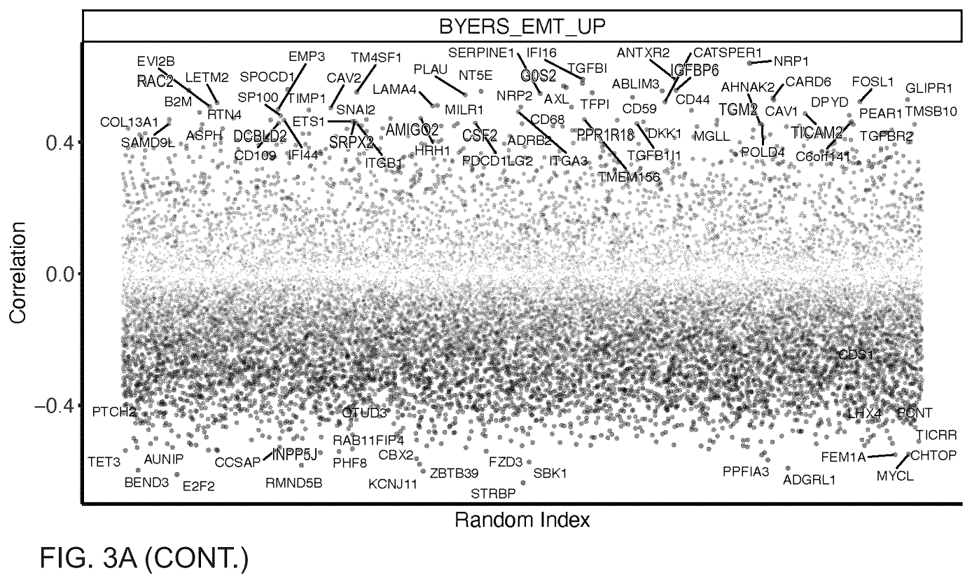

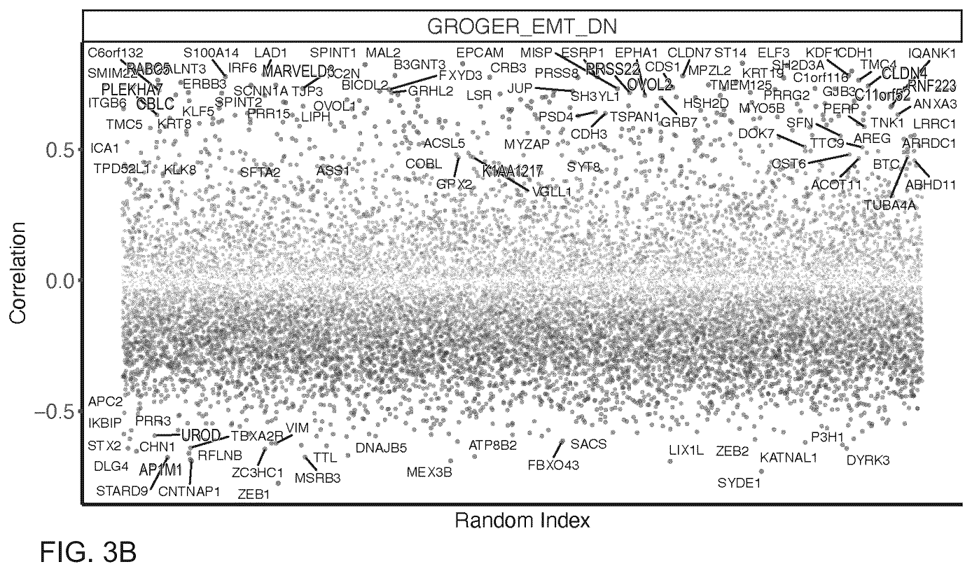

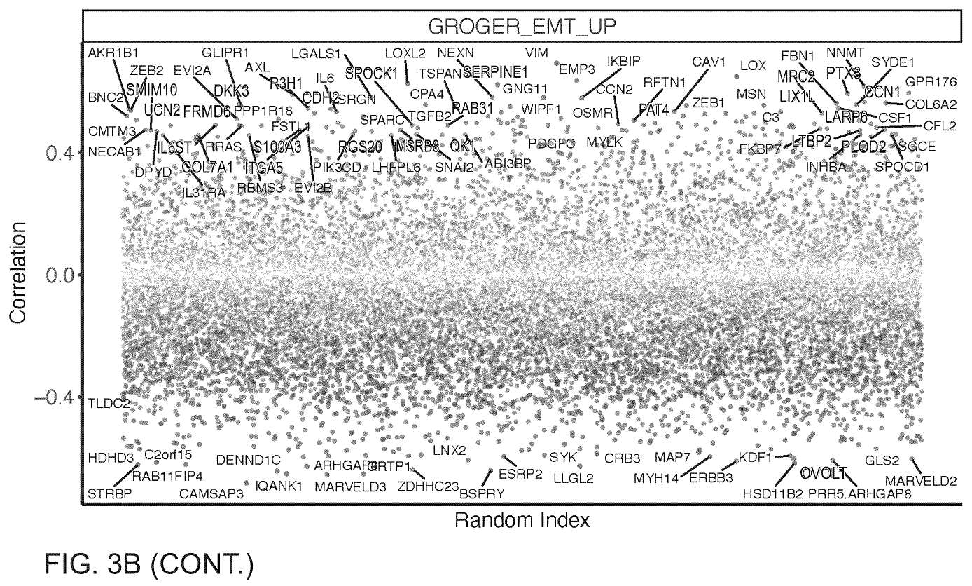

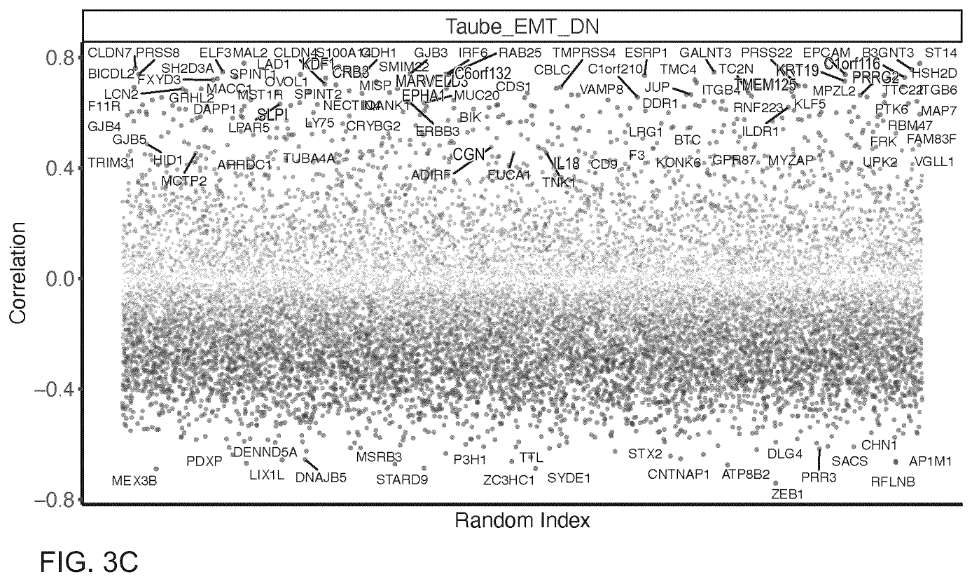

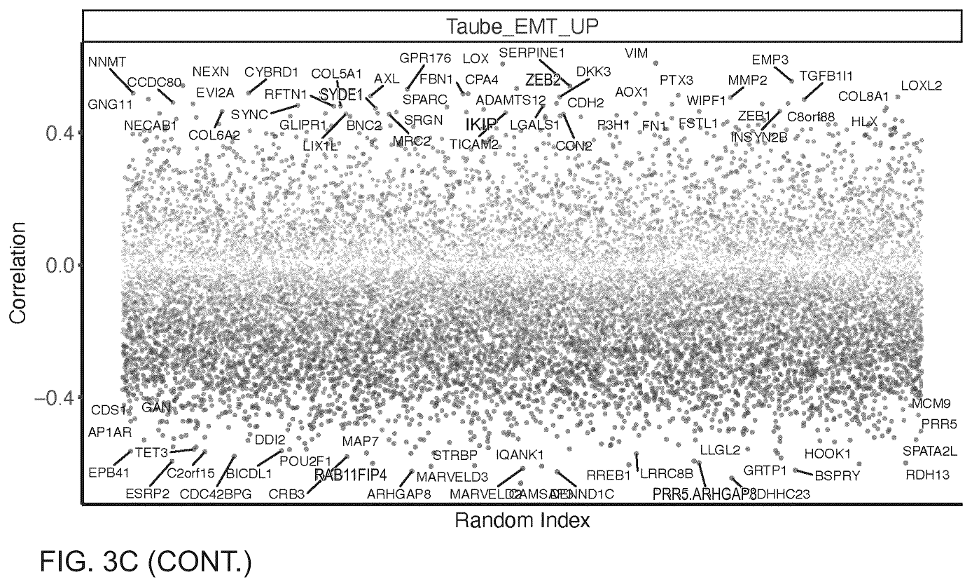

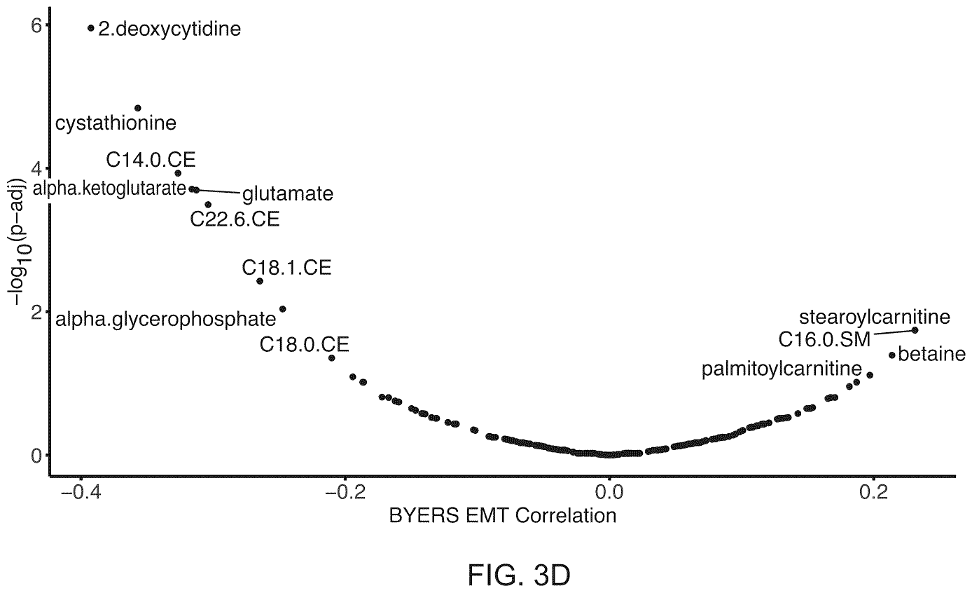

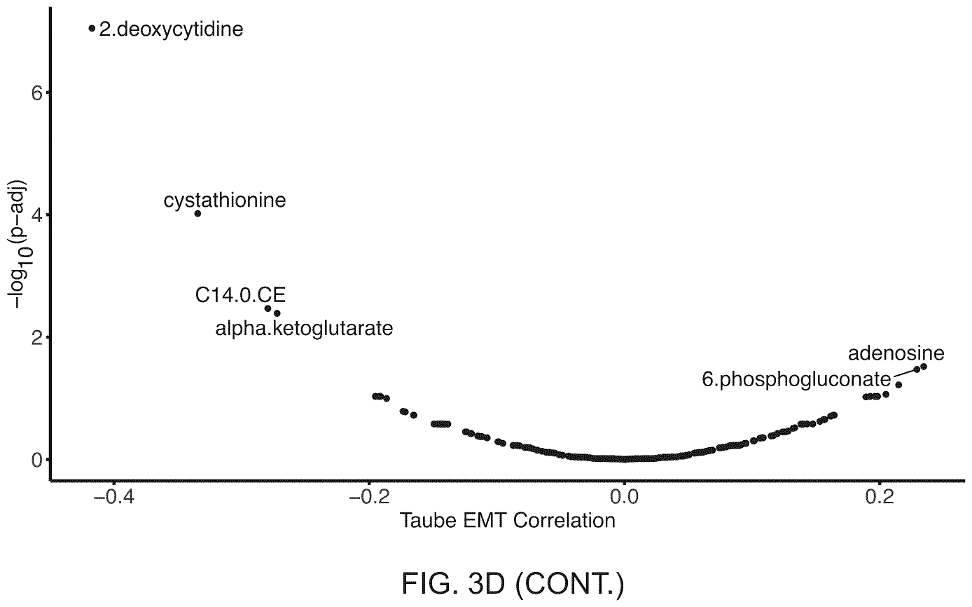

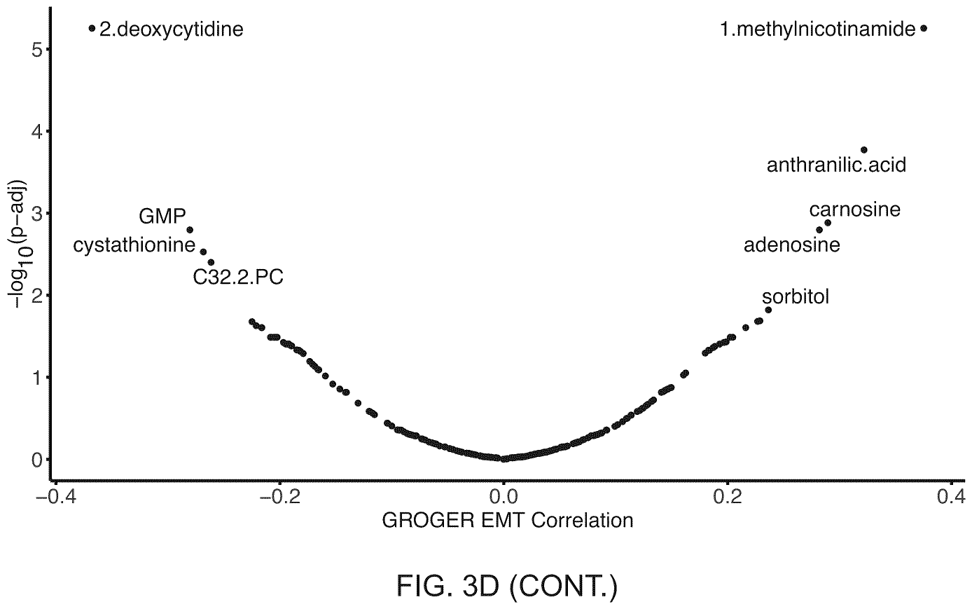

In example embodiments, the expression of one or more genes indicates a higher EMT score (i.e., down or up) and a cancer that can be treated as described herein (e.g., treating a tumor that is more mesenchymal or has an EMT up signature). In example embodiments, higher expression of positively correlated genes indicates a higher EMT score (see, FIGS. 3A, 3B, and 3C). In example embodiments, the levels of one or more metabolites indicates a higher EMT score. In example embodiments, higher metabolites selected from stearoylcarnitine, C16.0.SM, betaine, palmitoylcarnitine, adenosine, 6.phosphogluconate, 1.methylnicotinamide, anthranilic.acid, carnosine, and sorbitol indicate a higher EMT score (see, FIG. 3D).

### Example Drugs

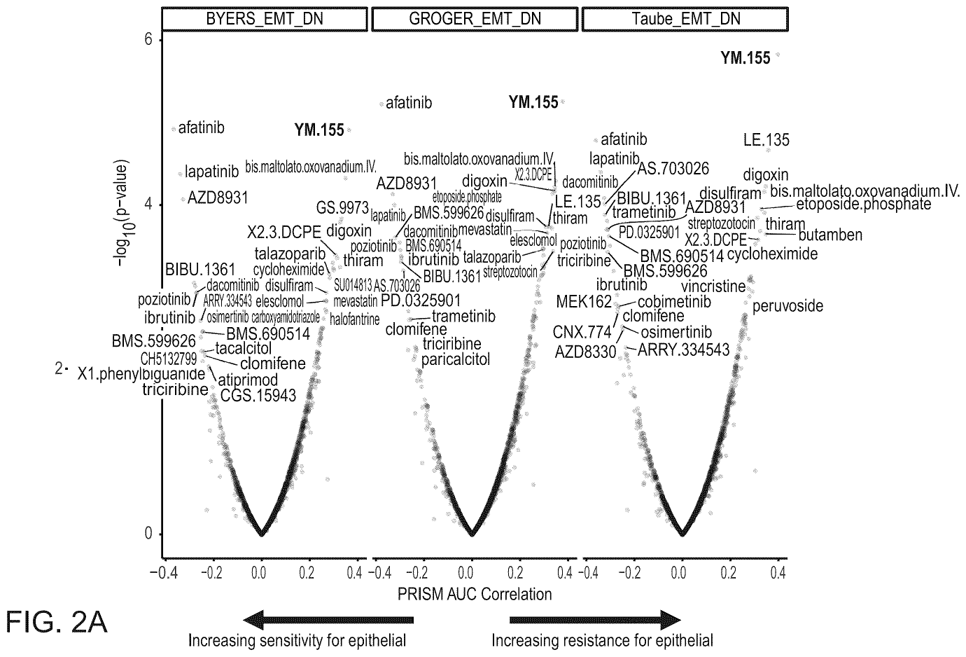

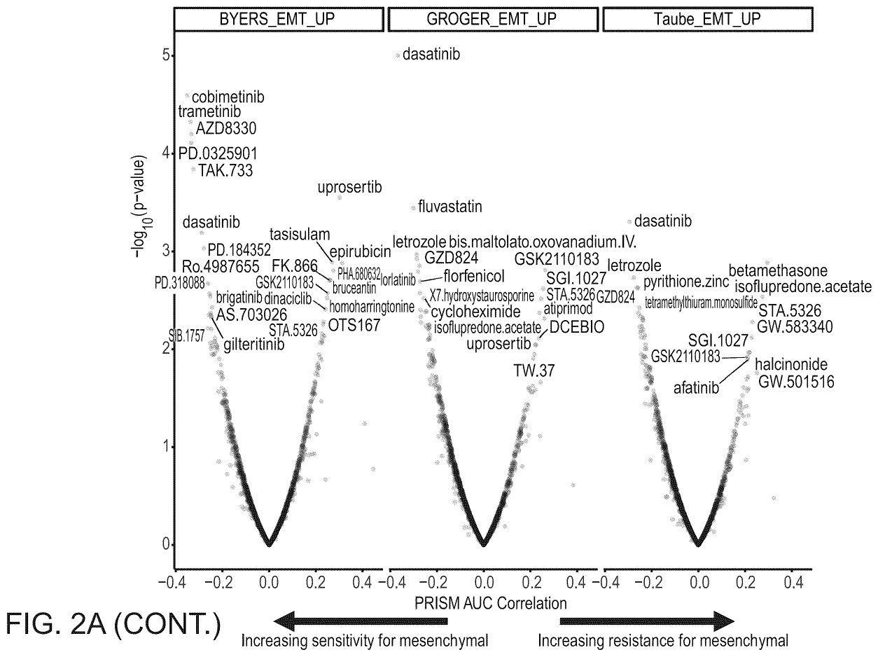

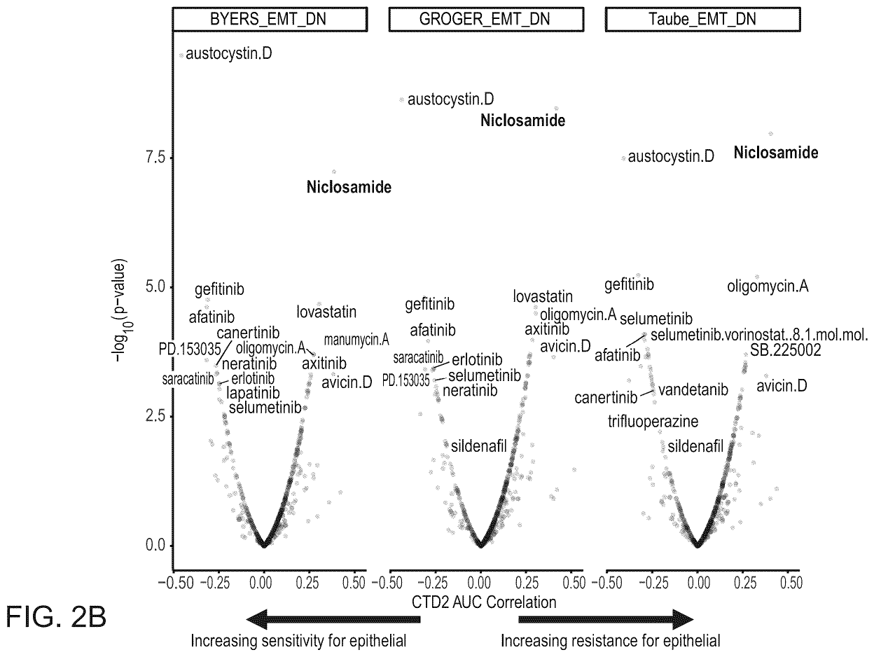

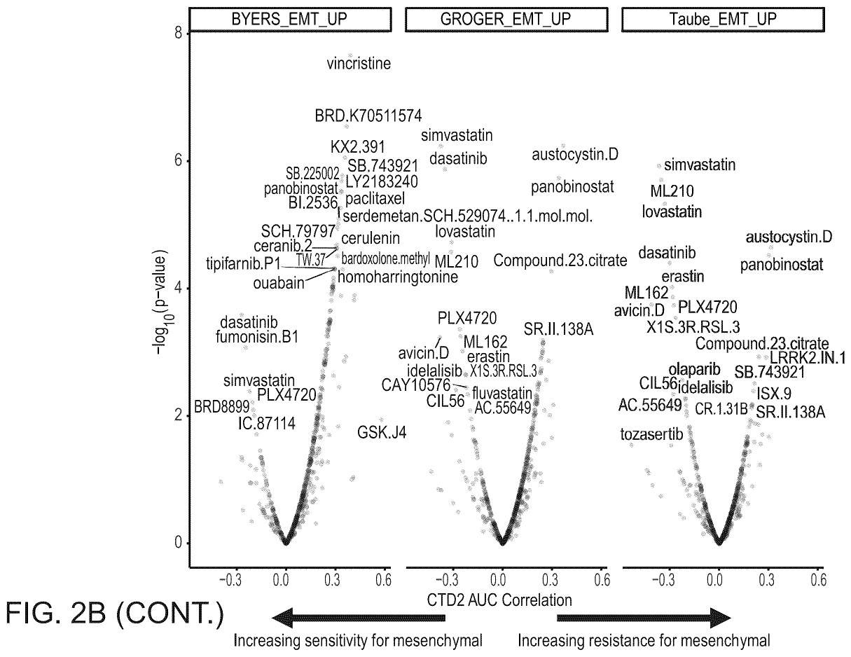

In example embodiments, drugs that therapeutically target the mesenchymal state in EMT cancers are used to treat the cancer. In example embodiments, the drug is one or more of any of the following drugs: YM-155, niclosamide, bis.maltolato.oxovanadium.IV (also known as, bis(maltolato)oxovanadium(IV); and 2-methyl-4-oxopyran-3-olate;oxovanadium(2+)), digoxin, GS.9973, LE.135 (also known as, LE-135; and 4-(5,7,7,10,10-pentamethyl-8,9-dihydronaphtho[2,3-b][1,4]benzodiazepin-13-yl)benzoic acid), thiram, butamben, talazoparib, cycloheximide, X2.3.DCPE, epirubicin, etoposide.phosphate, verubulin, indisulam, tasisulam, vinflunine, vindesine, dinaciclib, streptozotocin, Olaparib, PHA.680632, entinostat, SB.225002, oligomycin.A, ouabain, STF.31, lovastatin, LY2183240, BI.2536, SCH.79797, vincristine, homoharringtonine, tipifarnib, tivantinib, methotrexate, axitinib, BRD.K97651142, gossypol, and phloretin (see, e.g., FIG. 2). The indicated drugs were shown to be more lethal in tumor cells having a mesenchymal state, but may promote survival in tumor cells having an epithelial state (i.e., tumor cells having a mesenchymal state were sensitive to the drugs). Before the present disclosure, only considering a particular cancer that included both the epithelial and mesenchymal states (and any intermediate states), the drugs would not have been identified, in particular, drugs showing only a vulnerability in the mesenchymal state and not the epithelial state.

YM155 or YM-155 (Sepantronium Bromide; 1-(2-methoxyethyl)-2-methyl-3-(pyrazin-2-ylmethyl)benzo[f]benzimidazol-3-ium-4,9-dione;bromide) is a potent survivin suppressant by inhibiting Survivin promoter activity with IC50 of 0.54 nM in HeLa-SURP-luc and CHO-SV40-luc cells; does not significantly inhibit SV40 promoter activity, but is observed to slightly inhibit the interaction of Survivin with XIAP. YM155 down-regulates survivin and XIAP, modulates autophagy and induces autophagy-dependent DNA damage in breast cancer cells. YM 155 is a survivin suppressor. Down-regulates survivin expression and sensitizes human NSCLC cell lines to radiation. Enhances radiation-induced tumor regression in nude mice bearing NSCLC xenografts. Also induces apoptosis in TRAIL-resistant glioma cells in vitro.

Niclosamide (5-chloro-N-(2-chloro-4-nitrophenyl)-2-hydroxybenzamide) is a secondary carboxamide resulting from the formal condensation of the carboxy group of 5-chlorosalicylic acid with the amino group of 2-chloro-4-nitroaniline. It is an oral anthelmintic drug approved for use against tapeworm infections. It has a role as a piscicide, a molluscicide, an antiparasitic agent, an anticoronaviral agent, an anthelminthic drug, an apoptosis inducer and a STAT3 inhibitor. It is a member of monochlorobenzenes, a member of salicylanilides, a C-nitro compound, a secondary carboxamide and a member of benzamides. It is functionally related to a 5-chlorosalicylic acid. Niclosamide is also an orally bioavailable chlorinated salicylanilide, with anthelmintic and potential antineoplastic activity. Upon oral administration, niclosamide specifically induces degradation of the androgen receptor (AR) variant V7 (AR-V7) through the proteasome-mediated pathway. This downregulates the expression of the AR variant, inhibits AR-V7-mediated transcriptional activity, and reduces AR-V7 recruitment to the prostate-specific antigen (PSA) gene promoter. Niclosamide also prevents AR-V7-mediated STAT3 phosphorylation and activation. This inhibits AR/STAT3-mediated signaling and prevents expression of STAT3 target genes. Altogether, this may inhibit growth of AR-V7-overexpressing cancer cells. The AR-V7 variant, which is encoded by contiguous splicing of AR exons 1/2/3/CE3, is upregulated in a variety of cancer cell types, and is associated with both cancer progression and resistance to AR-targeted therapies.

Phloretin (3-(4-hydroxyphenyl)-1-(2,4,6-trihydroxyphenyl)propan-1-one) is a member of the class of dihydrochalcones that is dihydrochalcone substituted by hydroxy groups at positions 4, 2′, 4′ and 6′. It has a role as a plant metabolite and an antineoplastic agent. It is functionally related to a dihydrochalcone. Phloretin is a natural phenol which inhibits a variety of transporters. It inhibits the monocarboxylate transporters MCT1 and MCT2 (IC₅₀=28 and 14 μM, respectively), restricting the rapid transport of monocarboxylates like lactate and pyruvate across the plasma membrane. Phloretin also blocks the sodium/D-glucose cotransporter (Kᵢ=86 μM) and the human concentrative nucleoside transporter 3 (Kᵢ=32 μM).

Streptozotocin or streptozocin (INN, USP) (STZ; 2-Deoxy-2-({[methyl(nitroso)amino]carbonyl}amino)-β-D-glucopyranose) is a naturally occurring alkylating antineoplastic agent that is particularly toxic to the insulin-producing beta cells of the pancreas in mammals. It is used in medicine for treating certain cancers of the islets of Langerhans and used in medical research to produce an animal model for hyperglycemia and Alzheimer's in a large dose, as well as type 2 diabetes or type 1 diabetes with multiple low doses. Streptozotocin is approved by the U.S. Food and Drug Administration (FDA) for treating metastatic cancer of the pancreatic islet cells. Since it carries a substantial risk of toxicity and rarely cures the cancer, its use is generally limited to patients whose cancer cannot be removed by surgery. In these patients, streptozotocin can reduce the tumor size and reduce symptoms (especially hypoglycemia due to excessive insulin secretion by insulinomas).[1] A typical dose is 500 mg/m2/day by intravenous injection, for 5 days, repeated every 4-6 weeks. Due to its high toxicity to beta cells, in scientific research, streptozotocin has also been long used for inducing insulitis and diabetes on experimental animals.[2] Streptozotocin has also been used for modeling Alzheimer's disease through memory loss in mice.[3]

PHA-680632 (N-(2,6-diethylphenyl)-3-[[4-(4-methylpiperazin-1-yl)benzoyl]amino]-4,6-dihydro-1H-pyrrolo[3,4-c]pyrazole-5-carboxamide) is a novel and potent inhibitor of Aurora kinases, a small family of serine/threonine kinases regulating mitosis in chromosome segregation and cell division, that inhibits the activity of all three Aurora kinases with 50% inhibition concentration IC₅₀ values of 27 nM, 135 nM and 120 nM for Aurora A, B and C kinases respectively. PHA-680632 also cross-reacts with other kinases, including FGFR1, FLT3, LCK, PLK1, STLK2, VEGFR2 and VEGFR3, to a lesser extent and exhibits much higher IC50 values (390 to 5500 nM) compared to Aurora kinases. Moreover, PHA-680632 potently inhibits proliferation in a wide range of cancer cell types, including HCT116, A2780, HL60 and Hela cells, with IC₅₀ values ranging from 0.06 to 7.15 μM.

Gossypol (1,1′,6,6′,7,7′-Hexahydroxy-5,5′-diisopropyl-3,3′-dimethyl-2,2′-binaphthalene-8,8′-dicarbaldehyde) is an orally-active polyphenolic aldehyde with potential antineoplastic activity. Derived primarily from unrefined cottonseed oil, gossypol induces cell cycle arrest at the G0/G1 phase, thereby inhibiting DNA replication and inducing apoptosis. This agent also inhibits cell-signaling enzymes, resulting in inhibition of cell growth, and may act as a male contraceptive. Gossypol has been used in trials studying the treatment of Non-small Cell Lung Cancer.

BRD.K97651142 is an aphrocallistin derivative. Aphrocallistin has been isolated from the deep-water Hexactinellida sponge Aphrocallistes beatrix and inhibits the growth of a panel of human tumor cell lines with IC(50) values ranging from 7.5 to >100 μM and has been shown to induce G1 cell cycle arrest in the PANC-1 pancreatic carcinoma cell line (see, e.g., Wright A E, Roth G P, Hoffman J K, et al. Isolation, synthesis, and biological activity of aphrocallistin, an adenine-substituted bromotyramine metabolite from the Hexactinellida sponge Aphrocallistes beatrix. J Nat Prod. 2009; 72(6):1178-1183).

Axitinib (N-Methyl-2-[[3-[(E)-2-pyridin-2-ylethenyl]-1H-indazol-6-yl]sulfanyl]benzamide) is an orally bioavailable tyrosine kinase inhibitor. Axitinib inhibits the proangiogenic cytokines vascular endothelial growth factor (VEGF) and platelet-derived growth factor receptor (PDGF), thereby exerting an anti-angiogenic effect. Axitinib is an oral tyrosine kinase inhibitor selective for vascular endothelial growth factor (VEGF) receptors-1, -2 and -3 that is used in the therapy of advanced renal cell carcinoma.

Methotrexate ((2S)-2-[(4-{[(2,4-Diaminopteridin-6-yl)methyl](methyl)amino}benzoyl)amino]pentanedioic acid) is an antimetabolite and antifolate agent with antineoplastic and immunosuppressant activities. Methotrexate binds to and inhibits the enzyme dihydrofolate reductase, resulting in inhibition of purine nucleotide and thymidylate synthesis and, subsequently, inhibition of DNA and RNA syntheses. Methotrexate also exhibits potent immunosuppressant activity although the mechanism(s) of actions is unclear. Methotrexate is an antineoplastic and immunosuppressive agent widely used in the therapy of leukemia, lymphoma, solid tumors, psoriasis and rheumatoid arthritis.

Tivantinib ((3R,4R)-3-(5,6-Dihydro-4H-pyrrolo[3,2,1-ij]quinolin-1-yl)-4-(1H-indol-3-yl)-2,5-pyrrolidinedione) is an orally bioavailable small molecule inhibitor of c-Met with potential antineoplastic activity. c-Met inhibitor ARQ 197 binds to the c-Met protein and disrupts c-Met signal transduction pathways, which may induce cell death in tumor cells overexpressing c-Met protein or expressing constitutively activated c-Met protein. c-Met protein, the product of the proto-oncogene c-Met, is a receptor tyrosine kinase also known as hepatocyte growth factor receptor (HGFR); this protein is overexpressed or mutated in many tumor cell types and plays key roles in tumor cell proliferation, survival, invasion, and metastasis, and tumor angiogenesis.

Tipifarnib (R-115777; (+)-6-[amino(4-chlorophenyl)(1-methyl-1H-imidazol-5-yl)methyl]-4-(3-chlorophenyl)-1-methyl-2(1H)-quinolinone) is a substance that is being studied in the treatment of acute myeloid leukemia (AML) and other types of cancer. It belongs to the family of drugs called farnesyltransferase inhibitors. It is also called Zarnestra. Tipifarnib is a nonpeptidomimetic quinolinone with potential antineoplastic activity. Tipifarnib binds to and inhibits the enzyme farnesyl protein transferase, an enzyme involved in protein processing (farnesylation) for signal transduction. By inhibiting the farnesylation of proteins, this agent prevents the activation of Ras oncogenes, inhibits cell growth, induces apoptosis, and inhibits angiogenesis.

Omacetaxine mepesuccinate (formerly known as HHT or Homoharringtonine; 1-O-[(2S,3S,6R)-4-methoxy-16,18-dioxa-10-azapentacyclo[11.7.0.02,6.06,10.015,19]icosa-1(20),4,13,15(19)-tetraen-3-yl] 4-O-methyl (2R)-2-hydroxy-2-(4-hydroxy-4-methylpentyl)butanedioate) is a cephalotaxine-derived alkaloid ester obtained from Cephalotaxus harringtonia; used for the treatment of chronic or accelerated phase chronic myeloid leukemia. It has a role as an antineoplastic agent, a protein synthesis inhibitor, an apoptosis inducer and an anticoronaviral agent. It is an alkaloid ester, a tertiary alcohol, an organic heteropentacyclic compound and an enol ether. It is functionally related to a cephalotaxine.

Vincristine (methyl (1R,9R,10S,11R,12R,19R)-11-acetyloxy-12-ethyl-4-[(13S,15S,17S)-17-ethyl-17-hydroxy-13-methoxycarbonyl-1,11-diazatetracyclo[13.3.1.04,12.05,10]nonadeca-4(12),5,7,9-tetraen-13-yl]-8-formyl-10-hydroxy-5-methoxy-8,16-diazapentacyclo[10.6.1.01,9.02,7.016,19]nonadeca-2,4,6,13-tetraene-10-carboxylate) is a natural alkaloid isolated from the plant Vinca rosea Linn. Vincristine binds irreversibly to microtubules and spindle proteins in S phase of the cell cycle and interferes with the formation of the mitotic spindle, thereby arresting tumor cells in metaphase. This agent also depolymerizes microtubules and may also interfere with amino acid, cyclic AMP, and glutathione metabolism; calmodulin-dependent Ca++-transport ATPase activity; cellular respiration; and nucleic acid and lipid biosynthesis. It is used (commonly as the corresponding sulfate salt) as a chemotherapy drug for the treatment of leukemia, lymphoma, myeloma, breast cancer and head and neck cancer.

SCH 79797 (3-N-cyclopropyl-7-[(4-propan-2-ylphenyl)methyl]pyrrolo[3,2-f]quinazoline-1,3-diamine) is a non-peptide antagonist of proteinase-activated receptor 1 (PAR1). It blocks binding of the high affinity thrombin receptor-activating peptide (haTRAP) (IC₅₀=70 nM) as well as platelet aggregation induced by either haTRAP or α-thrombin (IC₅₀=0.3 and 3 μM, respectively). SCH 79797 has no activity against PAR2, PAR4, or other receptors involved in platelet activation. It also blocks PAR1 activation on vascular smooth muscle cells and endothelial cells.

The Plk1 Inhibitor BI 2536 (4-[[(7R)-8-cyclopentyl-7-ethyl-5,6,7,8-tetrahydro-5-methyl-6-oxo-2-pteridinyl]amino]-3-methoxy-N-(1-methyl-4-piperidinyl)-benzamide) is a small molecule compound with potential antineoplastic activities. BI 2536 binds to and inhibits Polo-like kinase 1 (Plk1), resulting in mitotic arrest, disruption of cytokinesis, and apoptosis in susceptible tumor cell populations. Plk1, a serine/threonine-protein kinase, is a key regulator of multiple processes fundamental to mitosis and cell division. BI 2536 is under investigation in clinical trial NCT00376623 (Efficacy and Safety of BI 2536 in Advanced or Metastatic Non Small Cell Lung Cancer).

LY2183240 (N,N-dimethyl-5-[(4-biphenyl)methyl]tetrazole-1-carboxamide) is a potent, competitive inhibitor of anandamide uptake (IC₅₀=270 μM; Kᵢ=540 μM) and hydrolysis. It increases anandamide levels in rat cerebellum (ED₅₀=1.37 mg/kg) and displays dose-dependent efficacy (3-30 mg/kg) in several rodent models of persistent pain.

Lovastatin ([(1S,3R,7S,8S,8aR)-8-[2-[(2R,4R)-4-hydroxy-6-oxooxan-2-yl]ethyl]-3,7-dimethyl-1,2,3,7,8,8a-hexahydronaphthalen-1-yl] (2S)-2-methylbutanoate) is a lactone metabolite isolated from the fungus Aspergillus terreus with cholesterol-lowering and potential antineoplastic activities. Lovastatin is hydrolyzed to the active beta-hydroxyacid form, which competitively inhibits 3-hydroxyl-3-methylgutarylcoenzyme A (HMG-CoA) reductase, an enzyme involved in cholesterol biosynthesis. In addition, this agent may induce tumor cell apoptosis and inhibit tumor cell invasiveness, possibly by inhibiting protein farnesylation and protein geranylgeranylation, and may arrest cells in the G1 phase of the cell cycle. The latter effect sensitizes tumor cells to the cytotoxic effects of ionizing radiation.

Glucose transporter 1 (Glut1) is an inducible carrier of pentoses and hexoses, including glucose. STF-31 (4-[[[[4-(1,1-dimethylethyl)phenyl]sulfonyl]amino]methyl]-N-3-pyridinyl-benzamide) is an inhibitor of Glut1 (IC₅₀=˜1 μM) that blocks glucose uptake. It induces necrosis in cancer cells that lack the von Hippel-Lindau tumor suppressor gene, which overexpress Glut1. Although STF-31 binds Glut1, suggesting a direct effect, STF-31 also inhibits nicotinamide phosphoribosyltransferase, an enzyme that induces Glut1 expression. STF-31 is also toxic to human pluripotent stem cells (hPSCs) and can be used to selectively eliminate hPSCs from mixed cultures.

Ouabain (3-[(1R,3S,5S,8R,9S,10R,11R,13R,14S,17R)-1,5,11,14-tetrahydroxy-10-(hydroxymethyl)-13-methyl-3-[(2R,3R,4R,5R,6S)-3,4,5-trihydroxy-6-methyloxan-2-yl]oxy-2,3,4,6,7,8,9,11,12,15,16,17-dodecahydro-1H-cyclopenta[a]phenanthren-17-yl]-2H-furan-5-one) is a steroid hormone that is a multi-hydroxylated alpha-L-rhamnosyl cardenoloide. It binds to and inhibits the plasma membrane Na(+)/K(+)-ATPase (sodium pump). It has been isolated naturally from Strophanthus gratus. It has a role as an EC 3.6.3.9 (Na(+)/K(+)-transporting ATPase) inhibitor, an EC 3.6.3.10 (H(+)/K(+)-exchanging ATPase) inhibitor, an EC 2.3.3.1 [citrate (Si)-synthase] inhibitor, an EC 3.1.3.41 (4-nitrophenylphosphatase) inhibitor, a plant metabolite, a cardiotonic drug, an ion transport inhibitor and an anti-arrhythmia drug. It is a cardenolide glycoside, a steroid hormone, an alpha-L-rhamnoside, a 14beta-hydroxy steroid, a 5beta-hydroxy steroid and an 11alpha-hydroxy steroid. It is a conjugate acid of an ouabain(1−). Ouabain is used to produce rapid digitalization in acute congestive heart failure and is also recommended in treatment of atrial or nodal paroxysmal tachycardia and atrial flutter.

Oligomycins ((1R,4E,5′S,6S,6′S,7R,8S,10R,11R,12S,14R,15S,16R,18E,20E,22R,25S,27R,28S,29R)-22-ethyl-7,11,14,15-tetrahydroxy-6′-[(2R)-2-hydroxypropyl]-5′,6,8,10,12,14,16,28,29-nonamethyl-3′,4′,5′,6′-tetrahydro-3H,9H,13H-spiro[2,26-dioxabicyclo[23.3.1]nonacosa-4,18,20-triene-27,2′-pyran]-3,9,13-trione) are macrolides created by Streptomyces species that can be toxic to other organisms. Different oligomycin isomers are highly specific for the disruption of mitochondrial metabolism. Oligomycin A, a dominant analog of the isomers, is an inhibitor of mitochondrial F₁Fₒ ATP synthase that induces apoptosis in a variety of cell types (average GI₅₀=270 nM). Oligomycin A exhibits antifungal, antitumor, and nematocidal activities, but has poor solubility in water and other biocompatible solvents, which limits its clinical application.

SB 225002 (1-(2-bromophenyl)-3-(2-hydroxy-4-nitrophenyl)urea; SB-225002) is a selective non-peptide inhibitor of CXCR2, inhibiting IL-8 binding to CXCR2 with an IC₅₀ value of 22 nM. SB225002 inhibits neutrophil chemotaxis in response to IL-8 in vitro (IC₅₀=20 nM) and blocks neutrophil margination induced by IL-8 in vivo (IC₅₀=30 nM). Similarly, SB 225002 reduces neutrophil influx, the production of inflammatory mediators, and tissue damage in TNBS-induced colitis in mice.

Entinostat ((Pyridin-3-yl)methyl ({4-[(2-aminophenyl)carbamoyl]phenyl}methyl)carbamate) is a synthetic benzamide derivative with potential antineoplastic activity. Entinostat binds to and inhibits histone deacetylase, an enzyme that regulates chromatin structure and gene transcription. This agent appears to exert dose-dependent effects in human leukemia cells including cyclin-dependent kinase inhibitor 1A (p21/CIP1/WAF1)-dependent growth arrest and differentiation at low drug concentrations; a marked induction of reactive oxygen species (ROS); mitochondrial damage; caspase activation; and, at higher concentrations, apoptosis. In normal cells, cyclin-dependent kinase inhibitor 1A expression has been associated with cell-cycle exit and differentiation.

Olaparib (4-[(3-[(4-cyclopropylcarbonyl)piperazin-1-yl]carbonyl)-4-fluorophenyl]methyl(2H)phthalazin-1-one) is a small molecule inhibitor of the nuclear enzyme poly(ADP-ribose) polymerase (PARP) with potential chemosensitizing, radiosensitizing, and antineoplastic activities. Olaparib selectively binds to and inhibits PARP, inhibiting PARP-mediated repair of single strand DNA breaks; PARP inhibition may enhance the cytotoxicity of DNA-damaging agents and may reverse tumor cell chemoresistance and radioresistance. PARP catalyzes post-translational ADP-ribosylation of nuclear proteins and can be activated by single-stranded DNA breaks.

Dinaciclib ((S)-3-(((3-Ethyl-5-(2-(2-hydroxyethyl)piperidin-1-yl)pyrazolo[1,5-a]pyrimidin-7-yl)amino)methyl)pyridine 1-oxide) is a pyrazolo[1,5-a]pyrimidine with potential antineoplastic activity. Dinaciclib selectively inhibits cyclin dependent kinases CDK1, CDK2, CDK5, and CDK9; inhibition of CDK1 and CDK2 may result in cell cycle repression and tumor cell apoptosis.

Vindesine (methyl (5S,7S,9S)-9-[(2β,3β,4β,5α,12β,19α)-3-(aminocarbonyl)-3,4-dihydroxy-16-methoxy-1-methyl-6,7-didehydroaspidospermidin-15-yl]-5-ethyl-5-hydroxy-1,4,5,6,7,8,9,10-octahydro-2H-3,7-methanoazacycloundecino[5,4-b]indole-9-carboxylate) is a synthetic derivative of vinblastine, a naturally occurring vinca alkaloid. Vindesine binds to and stabilizes tubulin, thereby interrupting tubulin polymerization and preventing the formation of the mitotic spindle and cell division; treated cells are unable to undergo mitosis and are arrested in metaphase. This agent also disrupts macromolecular synthesis.

Vinflunine (methyl (2β,3β,4β,5α,12β,19α)-4-(acetyloxy)-15-[(4R,6R,8S)-4-(1,1-difluoroethyl)-8-(methoxycarbonyl)-1,3,4,5,6,7,8,9-octahydro-2,6-methanoazecino[4,3-b]indol-8-yl]-3-hydroxy-16-methoxy-1-methyl-6,7-didehydroaspidospermidine-3-carboxylate) is a bi-fluorinated derivative of the semi-synthetic vinca alkaloid vinorelbine with antitubulin, antineoplastic, and antiangiogenic activities. Vinflunine inhibits tubulin assembly without any stabilization of assembled microtubules at concentrations comparable to those of other vinca alkaloids such as vincristine, vinblastine and vinorelbine; this effect on microtubule dynamics results in cell cycle arrest in mitosis and apoptosis. Compared to other vinca alkaloids, this agent binds weakly to the vinca-binding site, indicating that vinflunine may exhibit reduced neurotoxicity.

Tasisulam (N-(5-bromothiophen-2-yl)sulfonyl-2,4-dichlorobenzamide) is an acyl-sulfonamide with potential antineoplastic activity. Selectively toxic towards tumor cells, tasisulam appears to induce tumor cell apoptosis by a mitochondrial-targeted mechanism involving the loss of mitochondrial membrane potential and induction of reactive oxygen species (ROS). In combination with an angiogenesis inhibitor, this agent may exhibit synergistic antiangiogenic activity.

Indisulam (N-(-3-chloro-7-indolyl)-1,4-benzenedisulfonamide) is a novel sulfonamide compound with potential antineoplastic activity. Indisulam inhibits cyclin-dependent kinases (CDK), which regulate cell cycle progression and are usually over-expressed in cancerous cells. Inhibition of CDK results in G1/S phase arrest of the cell cycle, and may lead to induction of apoptosis and inhibition of tumor cell proliferation. In addition, indisulam also inhibits carbonic anhydrases (CA), especially isoforms IX and XII that are involved in aqueous humor production and are highly overexpressed in some types of cancers. Inhibition of CA IX and XII results in interference with ion exchange and pH in hypoxic tumor tissue and preventing chemoresistance to weakly basic antineoplastic agents.

Verubulin (N-(4-methoxyphenyl)-N,2-dimethylquinazolin-4-amine) is a quinazoline derivative with potential antineoplastic activities. Verubulin binds to and inhibits tubulin polymerization and interrupts microtubule formation, resulting in disruption of mitotic spindle assembly, cell cycle arrest in the G2/M phase, and cell death. This agent is not a substrate for several subtypes of multidrug resistance ABC transporters, and may be useful for treating multidrug resistant tumors. In addition, as a vascular disrupting agent, verubulin disrupts tumor microvasculature specifically, which may result in acute ischemia and massive tumor cell death. In addition, verubulin is able to cross the blood-brain barrier and accumulate in the brain.

Etoposide Phosphate (4′-Demethylepipodophyllotoxin 9-[4,6-O—(R)-ethylidene-O-D-glucopyranoside], 4′ (dihydrogen phosphate)) is a phosphate salt of a semisynthetic derivative of podophyllotoxin. Etoposide binds to the enzyme topoisomerase II, inducing double-strand DNA breaks, inhibiting DNA repair, and resulting in decreased DNA synthesis and tumor cell proliferation. Cells in the S and G2 phases of the cell cycle are most sensitive to this agent.

Epirubicin ((8S,10S)-10-{[(2R,4S,5R,6S)-4-Amino-5-hydroxy-6-methyloxan-2-yl]oxy}-6,8,11-trihydroxy-8-(2-hydroxyacetyl)-1-methoxy-5,7,8,9,10,12-hexahydrotetracene-5,12-dione) is a 4′-epi-isomer of the anthracycline antineoplastic antibiotic doxorubicin. Epirubicin intercalates into DNA and inhibits topoisomerase II, thereby inhibiting DNA replication and ultimately, interfering with RNA and protein synthesis. This agent also produces toxic free-radical intermediates and interacts with cell membrane lipids causing lipid peroxidation.

2,3-DCPE (2,3-DCPE Hydrochloride; 1009555-55-8; 2,3-DCPEHCl; 2,3-DCPE; 2-[3-(2,3-dichlorophenoxy)propylamino]ethanol;hydrochloride) is a proapoptotic compound with selectivity for cancer cells versus normal human cells. It induces apoptosis in vitro in various cancer cell lines (IC₅₀=0.89-2.69 μM) more effectively than in normal human fibroblasts (IC₅₀=12.6 μM). 2,3-DCPE down-regulates Bcl-xL, however forced overexpression of Bcl-xL is sufficient to block 2,3-DCPE apoptosis induction. 2,3-DCPE can induce S-phase arrest, upregulate p21, and activate ERK.

Cycloheximide (4-{(2R)-2-[(1S,3S,5S)-3,5-Dimethyl-2-oxocyclohexyl]-2-hydroxyethyl}piperidine-2,6-dione) is a dicarboximide that is 4-(2-hydroxyethyl)piperidine-2,6-dione in which one of the hydrogens attached to the carbon bearing the hydroxy group is replaced by a 3,5-dimethyl-2-oxocyclohexyl group. It is an antibiotic produced by the bacterium Streptomyces griseus. It has a role as a bacterial metabolite, a protein synthesis inhibitor, a neuroprotective agent, an anticoronaviral agent and a ferroptosis inhibitor. It is a member of piperidones, a piperidine antibiotic, an antibiotic fungicide, a dicarboximide, a secondary alcohol and a cyclic ketone. It is functionally related to a piperidine-2,6-dione. Cycloheximide is used as a fungicide and as an anticancer drug.

Talazoparib ((8S,9R)-5-Fluoro-8-(4-fluorophenyl)-9-(1-methyl-1H-1,2,4-triazol-5-yl)-2,7,8,9-tetrahydro-3H-pyrido[4,3,2-de]phthalazin-3-one) is an orally bioavailable inhibitor of the nuclear enzyme poly(ADP-ribose) polymerase (PARP) with potential antineoplastic activity. Talazoparib selectively binds to PARP and prevents PARP-mediated DNA repair of single strand DNA breaks via the base-excision repair pathway. This enhances the accumulation of DNA strand breaks, promotes genomic instability and eventually leads to apoptosis. PARP catalyzes post-translational ADP-ribosylation of nuclear proteins that signal and recruit other proteins to repair damaged DNA and is activated by single-strand DNA breaks. Talazoparib was approved by the FDA for use in germline BRCA mutated, HER2 negative, locally advanced or metastatic breast cancer.

Butamben (n-butyl-p-aminobenzoic acid) is an amino acid ester resulting from the formal condensation of the carboxy group of 4-aminobenzoic acid with the hydroxy group of butan-1-ol. Its local anesthetic properties have been used for surface anesthesia of the skin and mucous membranes, and for relief of pain and itching associated with some anorectal disorders. It has a role as a local anesthetic. It is a benzoate ester, a substituted aniline, an amino acid ester and a primary amino compound. It is functionally related to a 4-aminobenzoic acid and a butan-1-ol. It is a conjugate base of a butamben(1+).

Thiram (dimethylcarbamothioylsulfanyl N,N-dimethylcarbamodithioate) is an organic disulfide that results from the formal oxidative dimerisation of N,N-dimethyldithiocarbamic acid. It is widely used as a fungicidal seed treatment. It has a role as an antibacterial drug, an antiseptic drug and an antifungal agrochemical. It contains a dimethyldithiocarbamate. It is functionally related to a dimethyldithiocarbamic acid.

Entospletinib (GS-9973; 6-(1H-Indazol-6-yl)-N-(4-morpholinophenyl)imidazo[1,2-a]pyrazin-8-amine) is an orally available inhibitor of spleen tyrosine kinase (Syk), with potential antineoplastic activity. Upon oral administration of entospletinib, this agent may inhibit the activity of Syk, which inhibits B-cell receptor (BCR) signaling and leads to an inhibition of tumor cell activation, migration, adhesion and proliferation. Syk, a non-receptor cytoplasmic, BCR-associated tyrosine kinase, is expressed in hematopoietic tissues and is often overexpressed in hematopoietic malignancies.

Digoxin (3-[(3S,5R,8R,9S,10S,12R,13S,14S,17R)-3-[(2R,4S,5S,6R)-5-[(2S,4S,5S,6R)-5-[(2S,4S,5S,6R)-4,5-dihydroxy-6-methyloxan-2-yl]oxy-4-hydroxy-6-methyloxan-2-yl]oxy-4-hydroxy-6-methyloxan-2-yl]oxy-12,14-dihydroxy-10,13-dimethyl-1,2,3,4,5,6,7,8,9,11,12,15,16,17-tetradecahydrocyclopenta[a]phenanthren-17-yl]-2H-furan-5-one) is a cardiac glycoside. Digoxin inhibits the sodium potassium adenosine triphosphatase (ATPase) pump, thereby increasing intracellular calcium and enhancing cardiac contractility. This agent also acts directly on the atrioventricular node to suppress conduction, thereby slowing conduction velocity. Apparently due to its effects on intracellular calcium concentrations, digoxin induces apoptosis of tumor cells via a pathway involving mitochondrial cytochrome c and caspases 8 and 3.

### Example Gene Targets

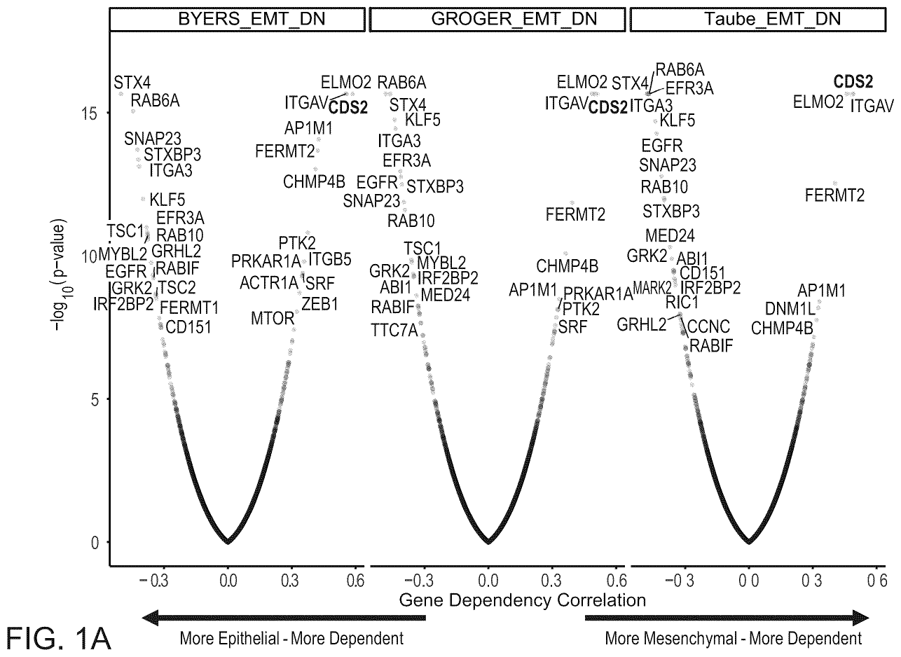

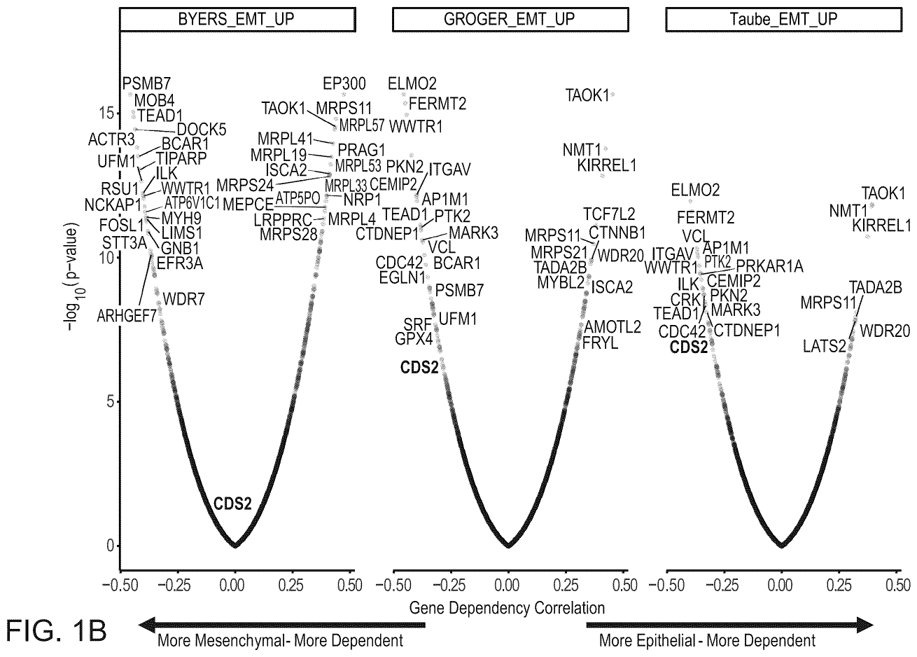

In example embodiments, genes therapeutically targeted or detected according to the present disclosure include genes expressed in tumor cells having a mesenchymal, drug resistant, state. In example embodiments, the mesenchymal state in EMT cancers is targeted using one or more agents targeting one or more genes that the mesenchymal tumor cells or tumor cells expressing a mesenchymal gene signature have a dependency on. Before the present disclosure, only considering a particular cancer that included both the epithelial and mesenchymal states (and any intermediate states), the gene targets would not have been identified, in particular, genes showing only a vulnerability in the mesenchymal state and not the epithelial state. In example embodiments, the one or more genes targeted are selected from CDS2, ELMO2, ITGAV, AP1M1, FERMT2, CHMP4B, PTK2, ITGB5, PRKAR1A, ACTR1A, SRF, ZEB1, MTOR, DNM1L PSMB7, MOB4, TEAD1, DOCK5, ACTR3, BCAR1, UFM1, TIPARP, ILK, RSU1, WWTR1, NCKAP1, ATP6V1C1, MYH9, FOSL1, LIMS1, GNB1, EFR3A, ARHGEF7, WDR7, FERMT2, PKN2, CEMIP2, TEAD1, CTDNEP1, MARK3, VCL, CDC42, BCAR1, EGLN1, UFM1, GPX4, and CRK (see, e.g., FIG. 1).

In example embodiments, CDS2 is targeted. As used herein CDS2 refers to the gene and gene product for CDP-Diacylglycerol Synthase 2 (also known as, Phosphatidate Cytidylyltransferase 2, CTP:Phosphatidate Cytidylyltransferase 2, CDP-Diglyceride Pyrophosphorylase 2, CDP-Diglyceride Synthase 2, CDP-DAG Synthase 2, CDP-DG Synthase 2, EC 2.7.7.41, CDS 2, CDP-Diacylglycerol Synthase (Phosphatidate Cytidylyltransferase) 2, CDP-Diglyceride Diphosphorylase 2, CDP-Diglyceride Synthetase 2, CDP-DG Synthetase 2, EC 2.7.7). Breakdown products of phosphoinositides are ubiquitous second messengers that function downstream of many G protein-coupled receptors and tyrosine kinases regulating cell growth, calcium metabolism, and protein kinase C activity. This gene encodes an enzyme which regulates the amount of phosphatidylinositol available for signaling by catalyzing the conversion of phosphatidic acid to CDP-diacylglycerol. This enzyme is an integral membrane protein localized to two subcellular domains, the matrix side of the inner mitochondrial membrane where it is thought to be involved in the synthesis of phosphatidylglycerol and cardiolipin and the cytoplasmic side of the endoplasmic reticulum where it functions in phosphatidylinositol biosynthesis. Two genes encoding this enzyme have been identified in humans, one mapping to human chromosome 4q21 and a second to 20p13. In example embodiments, CDS2 is encoded for in NC_060944.1:5166426-5237294 Homo sapiens isolate CHM13 chromosome 20. Reference sequences for CDS2 include HGNC: 1801; NCBI Entrez Gene: 8760; Ensembl: ENSG00000101290; OMIM: 603549; and UniProtKB/Swiss-Prot: 095674. CDS2 catalyzes the conversion of phosphatidic acid (PA) to CDP-diacylglycerol (CDP-DAG), an essential intermediate in the synthesis of phosphatidylglycerol, cardiolipin and phosphatidylinositol (D'Souza K, Kim Y J, Balla T, Epand R M. Distinct properties of the two isoforms of CDP-diacylglycerol synthase. Biochemistry. 2014; 53(47):7358-7367). CDS2 is selective for the acyl chains at the sn-1 and sn-2 positions, the most preferred species being 1-stearoyl-2-arachidonoyl-sn-phosphatidic acid. Id. Inhibition of CDS2 by phosphatidylinositol is acyl chain-dependent, with the strongest inhibition seen with the 1-stearoyl-2-arachidonoyl species. Id. Both CDS1 and CDS2 are inhibited by their anionic phospholipid end products, with phosphatidylinositol-(4,5)-bisphosphate showing the strongest inhibition. Id. In example embodiments, cancer is treated by a method comprising inhibiting CDS2 by administering an antagonistic analog of 1-stearoyl-2-arachidonoyl-sn-phosphatidic acid, or an anionic phospholipid end product or analog thereof, such as a 1-stearoyl-2-arachidonoyl species or phosphatidylinositol-(4,5)-bisphosphate or derivatives thereof. By “analog” is meant a molecule that is not identical, but has analogous functional or structural features. For example, analogs capable of binding to an enzyme binding site, but that cannot be processed by the enzyme. In chemistry, a derivative is a compound that is derived from a similar compound by a chemical reaction. Derivative may also refer to a compound that can be imagined to arise from another compound, if one atom or group of atoms is replaced with another atom or group of atoms (i.e., structural analog).

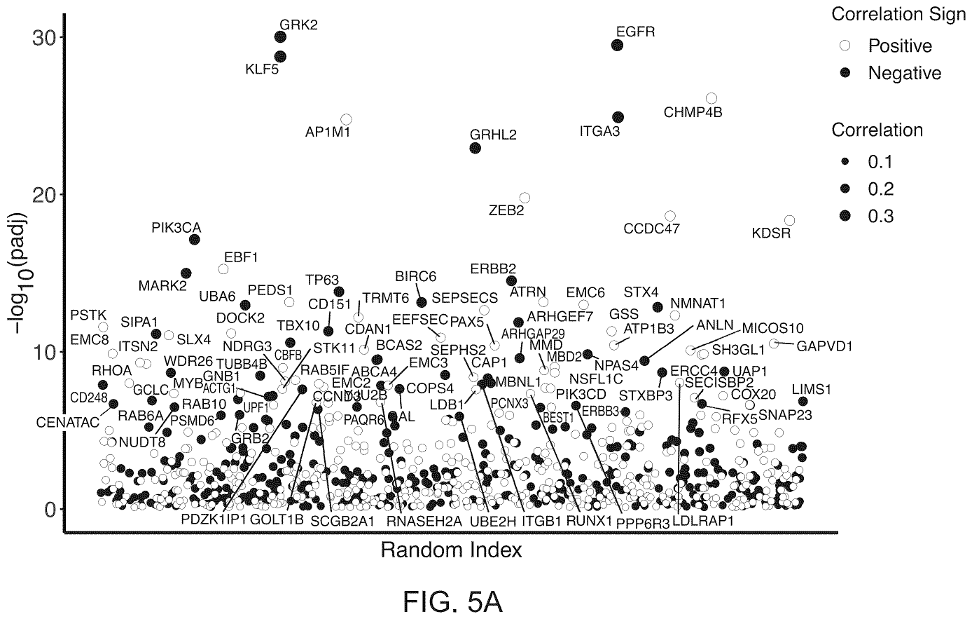

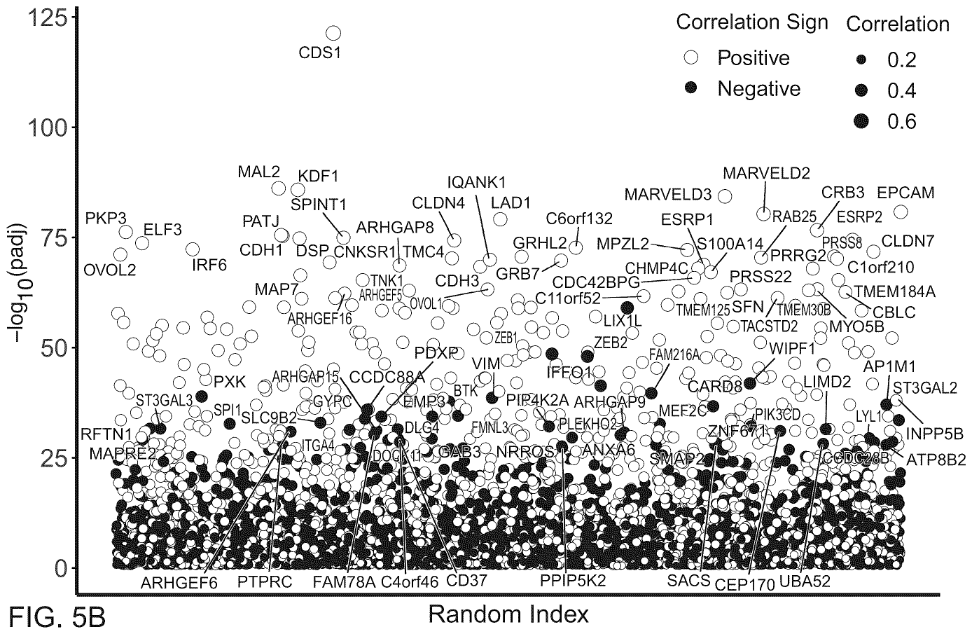

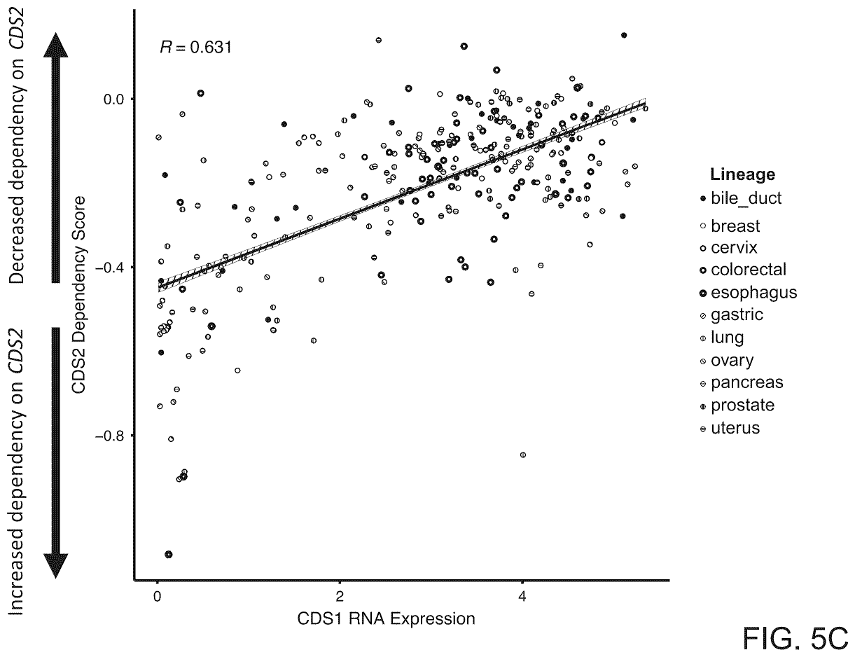

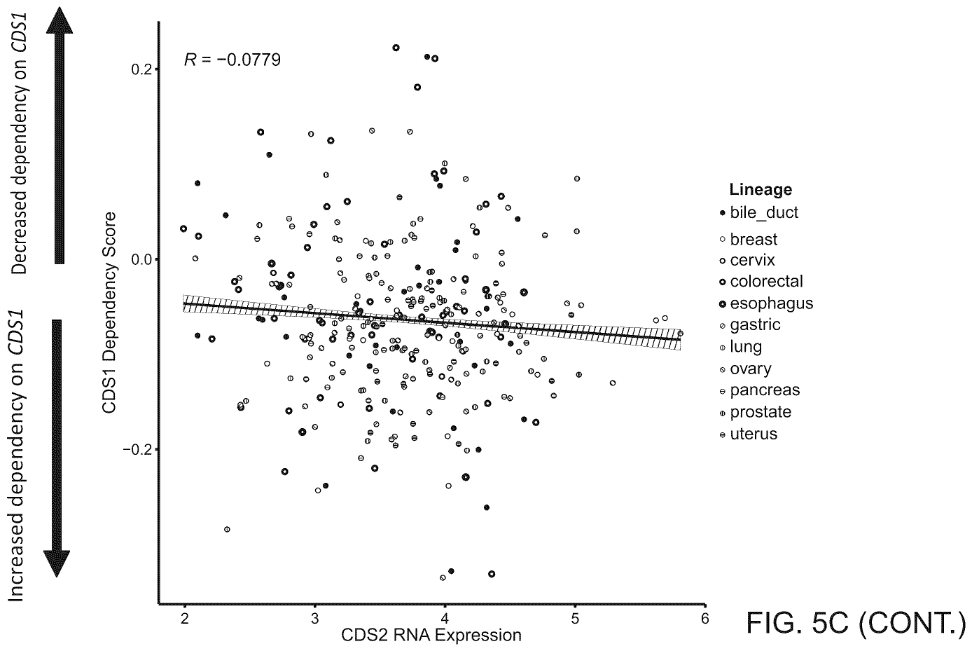

In example embodiments, CDS2 dependency is targeted and includes detecting expression of correlated genes. In example embodiments, tumor cells having a dependency on CDS2 express specific genes that are correlated with CDS2 dependency (see, e.g., FIG. 5B). In example embodiments, positively correlated genes in FIG. 5B mean that lower expression of these genes is correlated with increased dependency on CDS2. Thus, the lower expression of these genes the more the mesenchymal state is dependent upon CDS2. In other words, the cells are more vulnerable to a CDS2 inhibitor. In example embodiments, CDS1 is the most positively correlated gene with CDS2 dependency. As used herein CDS1 refers to CDP-Diacylglycerol Synthase 1. Diseases associated with CDS1 include Muscular Dystrophy, Congenital, With Cataracts And Intellectual Disability and Polymicrogyria, Bilateral Temporooccipital. Among its related pathways are Glycerophospholipid biosynthesis and Metabolism. Gene Ontology (GO) annotations related to this gene include transferase activity, transferring phosphorus-containing groups and phosphatidate cytidylyltransferase activity. An important paralog of this gene is CDS2. In example embodiments, CDS1 is encoded for in NC_000004.12:84583127-84651334 Homo sapiens chromosome 4. Reference sequences for CDS1 include HGNC: 1800; NCBI Entrez Gene: 1040; Ensembl: ENSG00000163624; OMIM®: 603548; and UniProtKB/Swiss-Prot: Q92903.

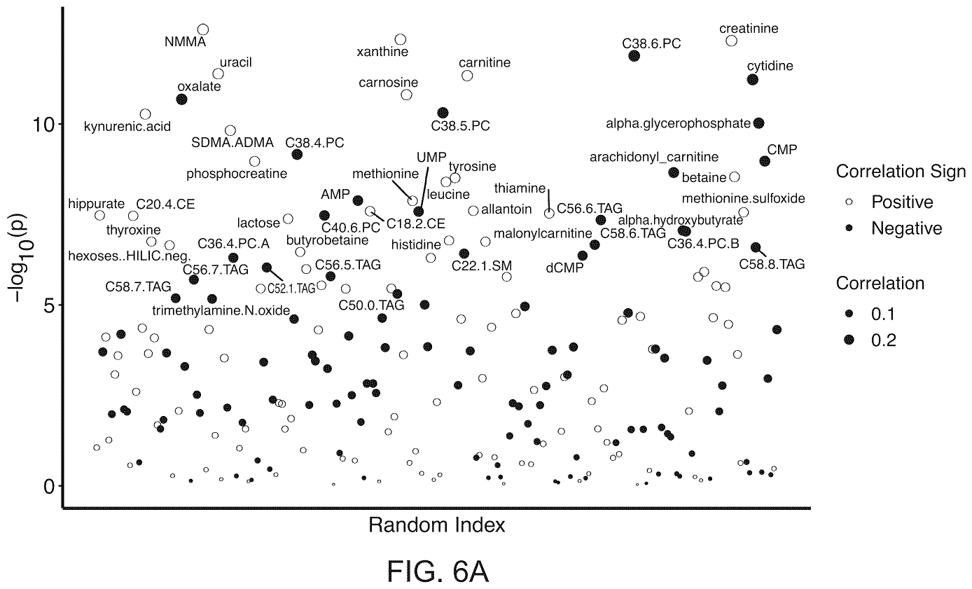

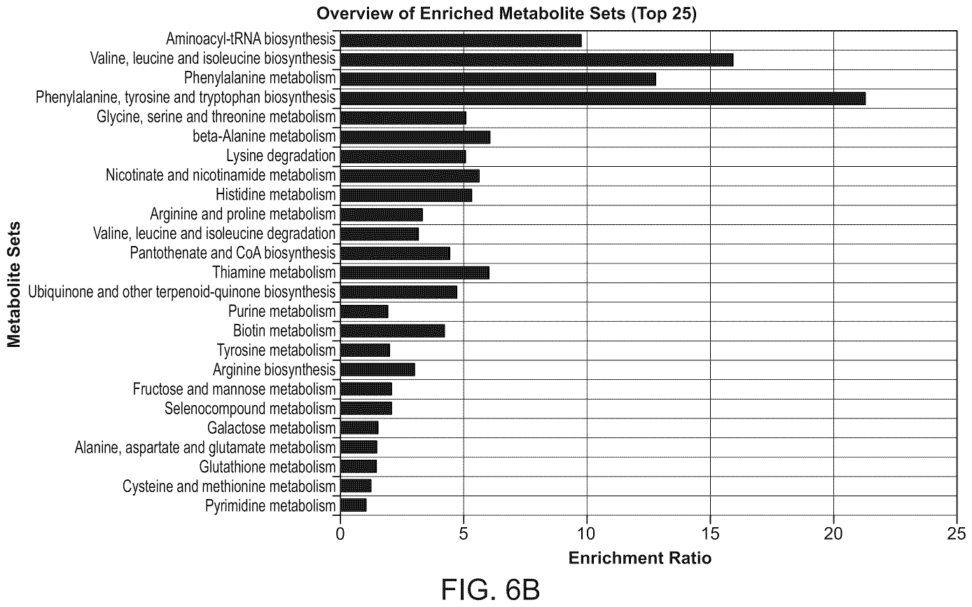

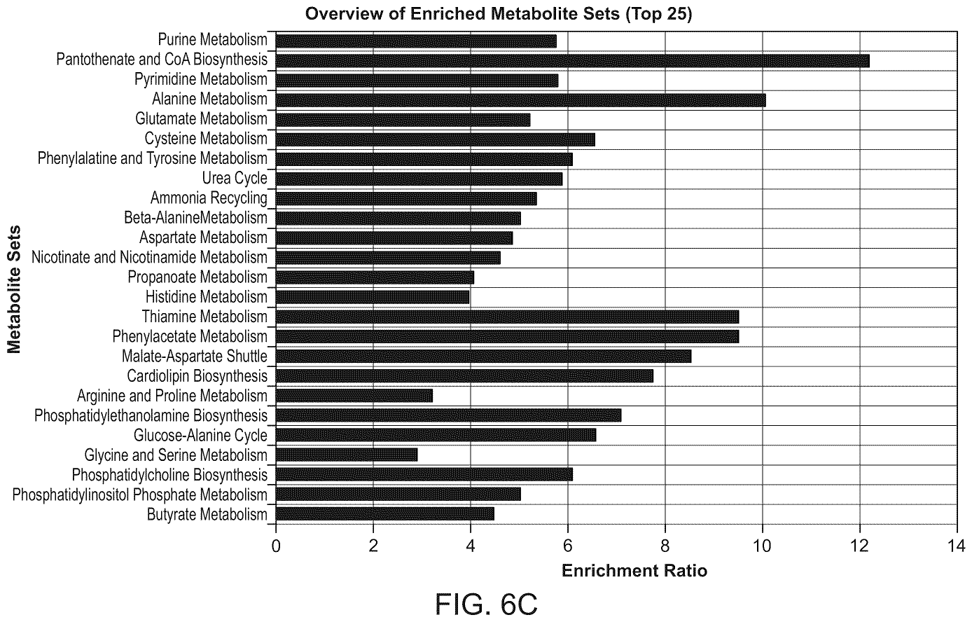

In example embodiments, CDS2 dependency is targeted and includes detecting levels of correlated metabolites. In example embodiments, tumor cells having a dependency on CDS2 express specific metabolites (see, e.g., FIG. 6A). In example embodiments, negatively correlated metabolites in FIG. 6A mean that higher levels of these metabolites is correlated with increased dependency on CDS2. Thus, the higher levels of these metabolites the more the mesenchymal state is dependent upon CDS2. In other words, the cells are more vulnerable to a CDS2 inhibitor.

In example embodiments, ELMO2 is targeted. As used herein ELMO2 refers to the gene and gene product for engulfment and cell motility 2 (also known as, VMPI; CED12; CED-12; ELMO-2; Ced-12A). ELMO2 (Engulfment And Cell Motility 2) is a Protein Coding gene. Diseases associated with ELMO2 include Vascular Malformation, Primary Intraosseous and Ramon Syndrome. Among its related pathways are Regulation of actin dynamics for phagocytic cup formation and Signaling by PTK6. Gene Ontology (GO) annotations related to this gene include binding and receptor tyrosine kinase binding. An important paralog of this gene is ELMO1. ELMO2 interacts with the dedicator of cytokinesis 1 protein. ELMO2 may function in phagocytosis of apoptotic cells and in cell migration. Alternative splicing results in multiple transcript variants. ELMO2 is involved in cytoskeletal rearrangements required for phagocytosis of apoptotic cells and cell motility. ELMO2 acts in association with DOCK1 and CRK. ELMO2 was proposed to be required in complex with DOCK1 to activate Rac Rho small GTPases. ELMO2 may enhance the guanine nucleotide exchange factor (GEF) activity of DOCK1. Interacts with the SH3-domain of DOCK1 via its SH3-binding site. Probably part of a complex with DOCK1 and RAC1. Probably part of a complex with DOCK1 and CRK isoform CRK-II. Interacts with ARHGEF16, DOCK4 and EPHA2; mediates activation of RAC1 by EPHA2 (Hiramoto-Yamaki N, Takeuchi S, Ueda S, et al. Ephexin4 and EphA2 mediate cell migration through a RhoG-dependent mechanism. J Cell Biol. 2010; 190(3):461-477). Interacts with ADGRB3 (Hamoud N, Tran V, Croteau L P, Kania A, Côté JF. G-protein coupled receptor BAI3 promotes myoblast fusion in vertebrates. Proc Natl Acad Sci USA. 2014; 111(10):3745-3750). Interacts with AUTS2. In example embodiments, ELMO2 is encoded for in NC_000020.11:c46406615-46366050 Homo sapiens chromosome 20. Reference sequences for ELMO2 include HGNC: 17233; NCBI Entrez Gene: 63916; Ensembl: ENSG00000062598; OMIM®: 606421; and UniProtKB/Swiss-Prot: Q96JJ3. In example embodiments, mesenchymal tumor cells are targeted by administering a Rac1 inhibitor (e.g., NSC23766, EHT1864, ZINC69391, 1A-116, 1D-142) (see, e.g., Arnst J L, Hein A L, Taylor M A, et al. Discovery and characterization of small molecule Rac1 inhibitors. Oncotarget. 2017; 8(21):34586-34600; and Liang J, Oyang L, Rao S, et al. Rac1, A Potential Target for Tumor Therapy. Front Oncol. 2021; 11:674426).

In example embodiments, ITGAV is targeted. As used herein ITGAV refers to the gene and gene product for integrin subunit alpha V (also known as, CD51; MSK8; VNRA; VTNR). The product of this gene belongs to the integrin alpha chain family. Integrins are heterodimeric integral membrane proteins composed of an alpha subunit and a beta subunit that function in cell surface adhesion and signaling. The encoded preproprotein is proteolytically processed to generate light and heavy chains that comprise the alpha V subunit. This subunit associates with beta 1, beta 3, beta 5, beta 6 and beta 8 subunits. The heterodimer consisting of alpha V and beta 3 subunits is also known as the vitronectin receptor. This integrin may regulate angiogenesis and cancer progression. Alternative splicing results in multiple transcript variants. Note that the integrin alpha 5 and integrin alpha V subunits are encoded by distinct genes. In example embodiments, ITGAV is encoded for in NC_000002.12:186590056-186680901 Homo sapiens chromosome 2. Reference sequences for ITGAV include HGNC: 6150; NCBI Entrez Gene: 3685; Ensembl: ENSG00000138448; OMIM®: 193210; and UniProtKB/Swiss-Prot: P06756. In example embodiments, mesenchymal tumor cells are targeted by administering a ITGAV inhibitor (see, e.g., Antithymocyte immunoglobulin, Levothyroxine, Abituzumab, Cilengitide, Etaracizumab, indomethacin) (see, e.g., Liu F, Wu Q, Han W, et al. Targeting integrin αvβ3 with indomethacin inhibits patient-derived xenograft tumour growth and recurrence in oesophageal squamous cell carcinoma. Clin Transl Med. 2021; 11(10):e548; Cheuk I W, Siu M T, Ho J C, Chen J, Shin V Y, Kwong A. ITGAV targeting as a therapeutic approach for treatment of metastatic breast cancer. Am J Cancer Res. 2020; 10(1):211-223; Yan W, Liu X, Wang Y, et al. Identifying Drug Targets in Pancreatic Ductal Adenocarcinoma Through Machine Learning, Analyzing Biomolecular Networks, and Structural Modeling. Front Pharmacol. 2020; 11:534).

In example embodiments, PTK2 is targeted. As used herein PTK2 refers to the gene and gene product for protein tyrosine kinase 2 (also known as, FAK; FADK; FAK1; FRNK; FADK 1; PPP1R71; p125FAK; pp125FAK). PTK2 (Protein Tyrosine Kinase 2) is a Protein Coding gene. Diseases associated with PTK2 include Malignant Pleural Mesothelioma and Ovarian Cancer. Among its related pathways are Regulation of actin dynamics for phagocytic cup formation and Integrin Pathway. Gene Ontology (GO) annotations related to this gene include transferase activity, transferring phosphorus-containing groups and protein tyrosine kinase activity. An important paralog of this gene is PTK2B. This gene encodes a cytoplasmic protein tyrosine kinase which is found concentrated in the focal adhesions that form between cells growing in the presence of extracellular matrix constituents. The encoded protein is a member of the FAK subfamily of protein tyrosine kinases but lacks significant sequence similarity to kinases from other subfamilies. Activation of this gene may be an important early step in cell growth and intracellular signal transduction pathways triggered in response to certain neural peptides or to cell interactions with the extracellular matrix. Several transcript variants encoding different isoforms have been found for this gene. Non-receptor protein-tyrosine kinase that plays an essential role in regulating cell migration, adhesion, spreading, reorganization of the actin cytoskeleton, formation and disassembly of focal adhesions and cell protrusions, cell cycle progression, cell proliferation and apoptosis. In example embodiments, PTK2 is encoded for in NC_000008.11:c141002079-140657900 Homo sapiens chromosome 8. Reference sequences for ITGAV include HGNC: 9611; NCBI Entrez Gene: 5747; Ensembl: ENSG00000169398; OMIM®: 600758; and UniProtKB/Swiss-Prot: Q05397. In example embodiments, mesenchymal tumor cells are targeted by administering a PTK2 inhibitor (see, e.g., Fostamatinib, Entrectinib, Pazopanib, PF-573,228 (PF-228), PF-562,271 (PF-271), NVP-226, Y15 (1,2,4,5-benzenetetraamine tetrahydrochloride), PND-1186, GSK2256098, PF-573,228, VS-6062 (PF 562,271), VS-6063 (PF-04554878 defactinib), VS-4718 (PND-1186), VS-6063, 2-({5-CHLORO-2-[(2-METHOXY-4-MORPHOLIN-4-YLPHENYL)AMINO]PYRIMIDIN-4-YL}AMINO)-N-METHYLBENZAMIDE, and 7-PYRIDIN-2-YL-N-(3,4,5-TRIMETHOXYPHENYL)-7H-PYRROLO[2,3-D]PYRIMIDIN-2-AMINE).

In example embodiments, ITGB5 is targeted. As used herein ITGB5 refers to the gene and gene product for integrin subunit beta 5. ITGB5 (Integrin Subunit Beta 5) is a Protein Coding gene. Diseases associated with ITGB5 include Villous Adenocarcinoma and Osteopetrosis, Autosomal Dominant 2. Among its related pathways are Apoptotic Pathways in Synovial Fibroblasts and Integrin Pathway. Gene Ontology (GO) annotations related to this gene include signaling receptor activity and integrin binding. An important paralog of this gene is ITGB3. This gene encodes a beta subunit of integrin, which can combine with different alpha chains to form a variety of integrin heterodimers. Integrins are integral cell-surface receptors that participate in cell adhesion as well as cell-surface mediated signaling. The alphav beta5 integrin is involved in adhesion to vitronectin. In example embodiments, ITGB5 is encoded for in NC_000003.12:c124901418-124761948 Homo sapiens chromosome 3. Reference sequences for ITGB5 include HGNC: 6160; NCBI Entrez Gene: 3693; Ensembl: ENSG00000082781; OMIM®: 147561; and UniProtKB/Swiss-Prot: P18084. In example embodiments, mesenchymal tumor cells are targeted by administering a ITGB5 inhibitor (see, e.g., Abituzumab, Cilengitide, GLPG-0187, Intetumumab).

In example embodiments, MTOR is targeted. As used herein MTOR refers to the gene and gene product for mechanistic target of rapamycin kinase. The protein encoded by this gene belongs to a family of phosphatidylinositol kinase-related kinases. These kinases mediate cellular responses to stresses such as DNA damage and nutrient deprivation. This kinase is a component of two distinct complexes, mTORC1, which controls protein synthesis, cell growth and proliferation, and mTORC2, which is a regulator of the actin cytoskeleton, and promotes cell survival and cell cycle progression. This protein acts as the target for the cell-cycle arrest and immunosuppressive effects of the FKBP12-rapamycin complex. Inhibitors of mTOR are used in organ transplants as immunosuppressants, and are being evaluated for their therapeutic potential in SARS-CoV-2 infections. Mutations in this gene are associated with Smith-Kingsmore syndrome and somatic focal cortical dysplasia type II. The ANGPTL7 gene is located in an intron of this gene. In example embodiments, MTOR is encoded for in NC_000001.11:c11262551-11106535 Homo sapiens chromosome 1. Reference sequences for MTOR include HGNC: 3942; NCBI Entrez Gene: 2475; Ensembl: ENSG00000198793; OMIM®: 601231; and UniProtKB/Swiss-Prot: P42345. In example embodiments, mesenchymal tumor cells are targeted by administering a MTOR inhibitor (see, e.g., rapamycin, temsirolimus, everolimus, dactolisib, zotarolimus, sirolimus, pimecrolimus).

In example embodiments, DNM1L is targeted. As used herein DNM1L refers to the gene and gene product for dynamin 1 like. DNM1L (Dynamin 1 Like) is a Protein Coding gene. Diseases associated with DNM1L include Encephalopathy Due To Defective Mitochondrial And Peroxisomal Fission 1 and Optic Atrophy 5. Among its related pathways are Programmed Cell Death and Apoptotic cleavage of cellular proteins. Gene Ontology (GO) annotations related to this gene include protein homodimerization activity and GTP binding. An important paralog of this gene is DNM2. This gene encodes a member of the dynamin superfamily of GTPases. The encoded protein mediates mitochondrial and peroxisomal division, and is involved in developmentally regulated apoptosis and programmed necrosis. Dysfunction of this gene is implicated in several neurological disorders, including Alzheimer's disease. Mutations in this gene are associated with the autosomal dominant disorder, encephalopathy, lethal, due to defective mitochondrial and peroxisomal fission (EMPF). Alternative splicing results in multiple transcript variants encoding different isoforms. In example embodiments, DNM1L is encoded for in NC_000012.12:32679301-32745650 Homo sapiens chromosome 12. Reference sequences for DNM1L include HGNC: 2973; NCBI Entrez Gene: 10059; Ensembl: ENSG00000087470; OMIM®: 603850; and UniProtKB/Swiss-Prot: 000429. In example embodiments, mesenchymal tumor cells are targeted by administering a DNM1L inhibitor (see, e.g., Dynasore, Dynazo) (see, also, Eschenburg S, Reubold T F. Modulation of dynamin function by small molecules. Biol Chem. 2018; 399(12):1421-1432).

In example embodiments, PSMB7 is targeted. As used herein PSMB7 refers to the gene and gene product for proteasome 20S subunit beta 7. The proteasome is a multicatalytic proteinase complex with a highly ordered ring-shaped 20S core structure. The core structure is composed of 4 rings of 28 non-identical subunits; 2 rings are composed of 7 alpha subunits and 2 rings are composed of 7 beta subunits. Proteasomes are distributed throughout eukaryotic cells at a high concentration and cleave peptides in an ATP/ubiquitin-dependent process in a non-lysosomal pathway. The encoded protein is a member of the proteasome B-type family, also known as the TiB family, and is a 20S core beta subunit in the proteasome. Expression of this catalytic subunit is downregulated by gamma interferon, and proteolytic processing is required to generate a mature subunit. A pseudogene of this gene is located on the long arm of chromosome 14. In example embodiments, PSMB7 is encoded for in NC_000009.12:c124415442-124353465 Homo sapiens chromosome 9. Reference sequences for PSMB7 include HGNC: 9544; NCBI Entrez Gene: 5695; Ensembl: ENSG00000136930; OMIM®: 604030; and UniProtKB/Swiss-Prot: Q99436. In example embodiments, mesenchymal tumor cells are targeted by administering a PSMB7 inhibitor (see, e.g., Oprozomib, Marizomib, Carfilzomib, Bortezomib).

In example embodiments, PKN2 is targeted. As used herein PKN2 refers to the gene and gene product for protein kinase N2. PKN2 (Protein Kinase N2) is a Protein Coding gene. Diseases associated with PKN2 include Toe Syndactyly, Telecanthus, And Anogenital And Renal Malformations and Bubonic Plague. Among its related pathways are Signaling by Rho GTPases and RHOC GTPase cycle. Gene Ontology (GO) annotations related to this gene include RNA binding and protein kinase activity. An important paralog of this gene is PKN1. PKC-related serine/threonine-protein kinase and Rho/Rac effector protein that participates in specific signal transduction responses in the cell. Plays a role in the regulation of cell cycle progression, actin cytoskeleton assembly, cell migration, cell adhesion, tumor cell invasion and transcription activation signaling processes. Phosphorylates CTTN in hyaluronan-induced astrocytes and hence decreases CTTN ability to associate with filamentous actin. Phosphorylates HDAC5, therefore lead to impair HDAC5 import. Direct RhoA target required for the regulation of the maturation of primordial junctions into apical junction formation in bronchial epithelial cells. Required for G2/M phases of the cell cycle progression and abscission during cytokinesis in a ECT2-dependent manner. Stimulates FYN kinase activity that is required for establishment of skin cell-cell adhesion during keratinocytes differentiation. Regulates epithelial bladder cells speed and direction of movement during cell migration and tumor cell invasion. Inhibits Akt pro-survival-induced kinase activity. Mediates Rho protein-induced transcriptional activation via the c-fos serum response factor (SRF). Involved in the negative regulation of ciliogenesis. In example embodiments, PKN2 is encoded for in NC_000001.11:88684273-88836255 Homo sapiens chromosome 1. Reference sequences for PKN2 include HGNC: 9406; NCBI Entrez Gene: 5586; Ensembl: ENSG00000065243; OMIM®: 602549; and UniProtKB/Swiss-Prot: Q16513. In example embodiments, mesenchymal tumor cells are targeted by administering a PKN2 inhibitor (see, e.g., Fostamatinib, Dasatinib, Entrectinib, Erlotinib).

In example embodiments, MARK3 is targeted. As used herein MARK3 refers to the gene and gene product for microtubule affinity regulating kinase 3. MARK3 (Microtubule Affinity Regulating Kinase 3) is a Protein Coding gene. Diseases associated with MARK3 include Visual Impairment And Progressive Phthisis Bulbi and Peutz-Jeghers Syndrome. Among its related pathways are Signaling downstream of RAS mutants and RAF/MAP kinase cascade. Gene Ontology (GO) annotations related to this gene include transferase activity, transferring phosphorus-containing groups and protein tyrosine kinase activity. An important paralog of this gene is MARK1. The protein encoded by this gene is activated by phosphorylation and in turn is involved in the phosphorylation of tau proteins MAP2 and MAP4. Several transcript variants encoding different isoforms have been found for this gene. In example embodiments, MARK3 is encoded for in NC_000014.9:103385415-103503831 Homo sapiens chromosome 14. Reference sequences for MARK3 include HGNC: 6897; NCBI Entrez Gene: 4140; Ensembl: ENSG00000075413; OMIM®: 602678; and UniProtKB/Swiss-Prot: P27448. In example embodiments, mesenchymal tumor cells are targeted by administering a MARK3 inhibitor (see, e.g., Fostamatinib, Alsterpaullone, Tamatinib, Cenisertib, CP-547632).

All gene name symbols refer to the gene as commonly known in the art. The examples described herein that refer to the human gene names are to be understood to also encompasses genes in any other organism, for example mouse genes or any other gene used in a model of disease (e.g., homologous, orthologous genes). Any reference to the gene symbol is a reference made to the entire gene or variants of the gene. Any reference to the gene symbol is also a reference made to the gene product (e.g., protein). The term, homolog, may apply to the relationship between genes separated by the event of speciation (e.g., ortholog). Orthologs are genes in different species that evolved from a common ancestral gene by speciation. Normally, orthologs retain the same function in the course of evolution. Gene symbols may be those referred to by the HUGO Gene Nomenclature Committee (HGNC) or National Center for Biotechnology Information (NCBI). A signature as described herein may encompass any of the genes described herein. The example DNA, RNA, and protein sequences can be used for the design of gene editing agents, antisense oligonucleotides (ASO), antibodies, small molecules, and RNAi.

### Standard of Care

In example embodiments, drugs specific for the mesenchymal state or agents targeting genes specific for the mesenchymal state (e.g., drug resistant tumor cells) are administered within the standard of care treatment, such that tumors resistant to the standard of care treatment are less or no longer resistant. Thus, the present disclosure encompasses combination therapies. In one embodiment, therapy comprising an agent targeting the mesenchymal state is administered within a standard of care where addition of the agent is synergistic within the steps of the standard of care. In one embodiment, the agent targets and/or shifts a tumor to a treatment responsive phenotype. The term “standard of care” as used herein refers to the current treatment that is accepted by medical experts as a proper treatment for a certain type of disease and that is widely used by healthcare professionals. Standard of care is also called best practice, standard medical care, and standard therapy.

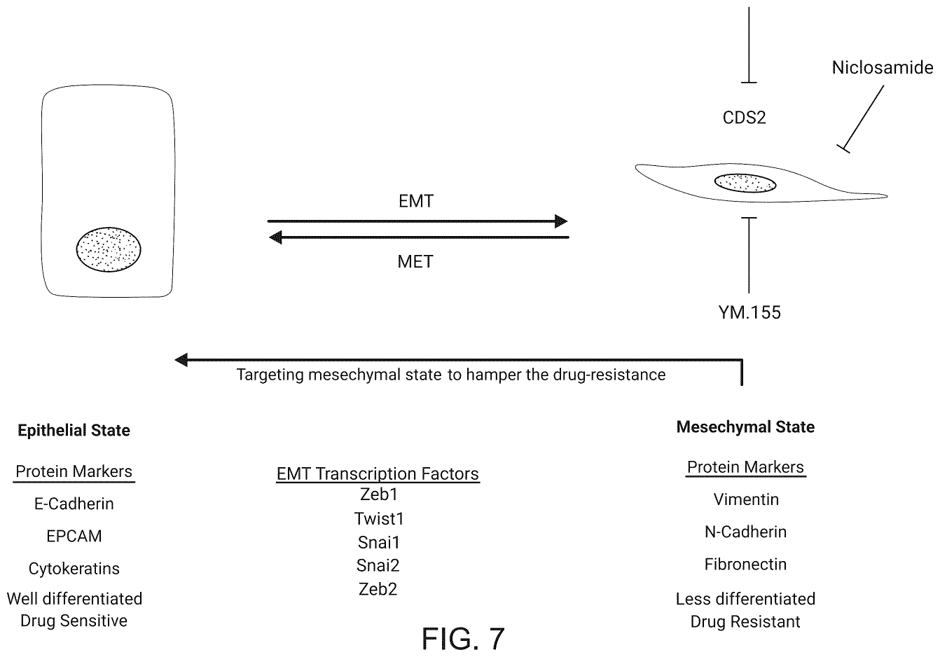

Standards of care for cancer generally include surgery, lymph node removal, radiation, chemotherapy, targeted therapies, antibodies targeting the tumor, and immunotherapy. Immunotherapy can include checkpoint blockers (CBP), chimeric antigen receptors (CARs), and adoptive T-cell therapy. As used herein, “ACT”, “adoptive cell therapy” and “adoptive cell transfer” may be used interchangeably. Adoptive cell therapy (ACT) can refer to the transfer of cells, most commonly immune-derived cells (e.g., T cells or NK cells), back into the same patient or into a new recipient host with the goal of transferring the immunologic functionality and characteristics into the new host. In example embodiments, ACT is used to target tumor cells having a mesenchymal cell state or epithelial state (e.g., see markers in FIG. 7). The adoptive transfer of autologous tumor infiltrating lymphocytes (TIL) (Zacharakis et al., (2018) Nat Med. 2018 June; 24(6):724-730; Besser et al., (2010) Clin. Cancer Res 16 (9) 2646-55; Dudley et al., (2002) Science 298 (5594): 850-4; and Dudley et al., (2005) Journal of Clinical Oncology 23 (10): 2346-57) or genetically re-directed peripheral blood mononuclear cells (Johnson et al., (2009) Blood 114 (3): 535-46; and Morgan et al., (2006) Science 314(5796) 126-9) has been used to successfully treat patients with advanced solid tumors, including melanoma, metastatic breast cancer and colorectal carcinoma, as well as patients with CD19-expressing hematologic malignancies (Kalos et al., (2011) Science Translational Medicine 3 (95): 95ra73). In certain embodiments, allogenic cells immune cells are transferred (see, e.g., Ren et al., (2017) Clin Cancer Res 23 (9) 2255-2266).

Various strategies may for example be employed to genetically modify T cells by altering the specificity of the T cell receptor (TCR) for example by introducing new TCR α and β chains with selected peptide specificity (see U.S. Pat. No. 8,697,854; PCT Patent Publications: WO2003020763, WO2004033685, WO2004044004, WO2005114215, WO2006000830, WO2008038002, WO2008039818, WO2004074322, WO2005113595, WO2006125962, WO2013166321, WO2013039889, WO2014018863, WO2014083173; U.S. Pat. No. 8,088,379).

As an alternative to, or addition to, TCR modifications, chimeric antigen receptors (CARs) may be used in order to generate immunoresponsive cells, such as T cells or natural killer cells (NK), specific for selected targets, such as malignant cells, with a wide variety of receptor chimera constructs having been described (see U.S. Pat. Nos. 5,843,728; 5,851,828; 5,912,170; 6,004,811; 6,284,240; 6,392,013; 6,410,014; 6,753,162; 8,211,422; and, PCT Publication WO9215322).

The standards of care for the most common cancers can be found on the website of National Cancer Institute (www.cancer.gov/cancertopics). A treatment clinical trial is a research study meant to help improve current treatments or obtain information on new treatments for patients with cancer. When clinical trials show that a new treatment is better than the standard treatment, the new treatment may be considered the new standard treatment. For example, standard of care for breast cancer generally includes surgery, radiation therapy, chemotherapy, hormone therapy, immunotherapy, and targeted therapy. Standard of care for bladder cancer generally includes surgery, radiation therapy, chemotherapy, immunotherapy, and targeted therapy. Standard of care for PDAC generally includes surgery (e.g., tumor resection or palliative surgery), radiation, chemotherapy, chemoradiation therapy (CRT), and targeted therapies (e.g., tyrosine kinase inhibitors, such as Tarceva (Erlotinib Hydrochloride), Sunitinib Malate, and Sutent (Sunitinib Malate); Olaparib). Standard of care for skin cancer generally includes surgery, chemotherapy, radiation therapy, immunotherapy, and targeted therapy.

In example embodiments, the standard of care includes chemotherapy. In the context of chemotherapy treatment, the mesenchymal cell states become enriched in the chemotherapy resistant population of cells left after completion of therapy. The chemotherapeutic agent cisplatin (CP) is widely used in the treatment of various types of cancer (see, e.g., Ashrafizadeh M, Zarrabi A, Hushmandi K, et al. Association of the Epithelial-Mesenchymal Transition (EMT) with Cisplatin Resistance. Int J Mol Sci. 2020; 21(11):4002). Resistance of cancer cells to CP chemotherapy has been extensively observed. Id. Among various reported mechanism(s), the epithelial-mesenchymal transition (EMT) process can significantly contribute to chemoresistance by converting the motionless epithelial cells into mobile mesenchymal cells and altering cell-cell adhesion as well as the cellular extracellular matrix, leading to invasion of tumor cells. Id.

Non-limiting examples of chemotherapy include Alkylating agents, such as, Altretamine, Bendamustine, Busulfan, Carboquone, Carmustine, Chlorambucil, Chlormethine, Chlorozotocin, Cyclophosphamide, Dacarbazine, Fotemustine, Ifosfamide, Lomustine, Melphalan, Melphalan flufenamide, Mitobronitol, Nimustine, Nitrosoureas, Pipobroman, Ranimustine, Semustine, Streptozotocin, Temozolomide, Thiotepa, Treosulfan, Triaziquone, Triethylenemelamine, Trofosfamide, and Uramustine; Anthracyclines, such as, Aclarubicin, Daunorubicin, Doxorubicin, Epirubicin, Idarubicin, Mitoxantrone, Pirarubicin, Valrubicin, and Zorubicin; Cytoskeletal disruptors (taxanes), such as, Abraxane, Cabazitaxel, Docetaxel, Larotaxel, Paclitaxel, Taxotere, and Tesetaxel; Epothilones, such as, Ixabepilone; Histone deacetylase inhibitors, such as, Vorinostat, and Romidepsin; Inhibitors of topoisomerase I, such as, Belotecan, Camptothecin, Exatecan, Gimatecan, Irinotecan, and Topotecan; Inhibitors of topoisomerase II, such as, Etoposide, Teniposide, and Tafluposide; Kinase inhibitors, such as, Bortezomib, Erlotinib, Gefitinib, Imatinib, Vemurafenib, and Vismodegib; Nucleotide analogs and precursor analogs, such as, Azacitidine, Azathioprine, Capecitabine, Cytarabine, Doxifluridine, Fluorouracil, Gemcitabine, Hydroxyurea, Mercaptopurine, Methotrexate, and Tioguanine (formerly Thioguanine); Peptide antibiotics, such as, Actinomycin, and Bleomycin; Platinum-based agents, such as, Carboplatin, Cisplatin, Dicycloplatin, Oxaliplatin, Nedaplatin, and Satraplatin; Retinoids, such as, Alitretinoin, Bexarotene, and Tretinoin; and alkaloids and derivatives, such as, Vinblastine, Vincristine, Vindesine, and Vinorelbine.

Non-limiting examples of chemotherapy also include Abraxane, Actinomycin, Alitretinoin, All-trans retinoic acid, Altretamine, Azacitidine, Azathioprine, Belotecan, Bendamustine, Bexarotene, Bleomycin, Bortezomib, Busulfan, Cabazitaxel, Camptothecin, Carboplatin, Carboquone, Carmustine, Capecitabine, Cisplatin, Chlorambucil, Chlormethine, Chlorozotocin, Cyclophosphamide, Cytarabine, Dacarbazine, Daunorubicin, Docetaxel, Doxifluridine, Doxorubicin, Epirubicin, Epothilone, Erlotinib, Etoposide, Exatecan, Fluorouracil, Fotemustine, Gefitinib, Gemcitabine, Gimatecan, Hydroxyurea, Idarubicin, Ifosfamide, Imatinib, Irinotecan, Ixabepilone, Larotaxel, Lomustine, Melphalan, Melphalan flufenamide, Mercaptopurine, Methotrexate, Mitobronitol, Mitomycin C, Mitoxantrone, Nimustine, Nitrosoureas, Oxaliplatin, Paclitaxel, Pemetrexed, Pipobroman, Ranimustine, Romidepsin, Semustine, Streptozotocin, Tafluposide, Taxotere, Temozolomide, Tesetaxel, Teniposide, Thiotepa, Tioguanine, Topotecan, Treosulfan, Tretinoin, Triaziquone, Triethylenemelamine, Valrubicin, Vemurafenib, Vinblastine, Vincristine, Vindesine, Vinorelbine, Vismodegib, and Vorinostat.

Non-limiting examples of common combination chemotherapy regimens include Cyclophosphamide, methotrexate, 5-fluorouracil, vinorelbine (CMF); Doxorubicin, cyclophosphamide (AC); Docetaxel, doxorubicin, cyclophosphamide (TAC); Doxorubicin, bleomycin, vinblastine, dacarbazine (ABVD); Mustine, vincristine, procarbazine, prednisolone (MOPP); Cyclophosphamide, doxorubicin, vincristine, prednisolone (CHOP, R-CVP); Bleomycin, etoposide, cisplatin (BEP); Epirubicin, cisplatin, 5-fluorouracil (ECF); Epirubicin, cisplatin, capecitabine (ECX); Methotrexate, vincristine, doxorubicin, cisplatin (MVAC); Cyclophosphamide, doxorubicin, vincristine, vinorelbine (CAV); 5-fluorouracil, folinic acid, oxaliplatin (FOLFOX); Gemcitabine, 5-fluorouracil (FOLFOX); and Doxorubicin, cisplatin, methotrexate, ifosfamide, etoposide (MAP/MAPIE).

In example embodiments, the standard of care includes targeted therapy. Targeted therapies may also be described as “chemotherapy” or “non-cytotoxic chemotherapy”, as “chemotherapy” strictly means only “treatment by chemicals”. Targeted therapy constitutes the use of agents specific for the deregulated proteins of cancer cells. The main categories of targeted therapy are currently small molecules and monoclonal antibodies. Small molecule targeted therapy drugs are generally inhibitors of enzymatic domains on mutated, overexpressed, or otherwise critical proteins within the cancer cell. Prominent examples are the tyrosine kinase inhibitors imatinib (Gleevec/Glivec) and gefitinib (Iressa). Other examples of small molecules include Gefitinib (Iressa, also known as ZD1839), Erlotinib, Sorafenib (Nexavar), Sunitinib (Sutent), Dasatinib (Sprycel), Lapatinib (Tykerb), Nilotinib (Tasigna), Bortezomib (Velcade), tamoxifen, Janus kinase inhibitors (e.g. tofacitinib), ALK inhibitors (e.g. crizotinib), Bcl-2 inhibitors (e.g. FDA approved venetoclax, obatoclax, navitoclax, and gossypol), PARP inhibitors (e.g. FDA approved olaparib, rucaparib, niraparib and talazoparib), PI3K inhibitors (e.g. perifosine), Apatinib, Zoptarelin doxorubicin (AN-152), Braf inhibitors (e.g., vemurafenib, dabrafenib, LGX818), MEK inhibitors (e.g., trametinib, MEK162) often in combination with BRAF inhibitors, CDK inhibitors, (e.g., PD-0332991, LEE011), Hsp90 inhibitors, Hedgehog pathway inhibitors (e.g., vismodegib and sonidegib), Salinomycin, VAL-083 (dianhydrogalactitol), Vintafolide, Temsirolimus (Torisel), Everolimus (Afinitor), Vemurafenib (Zelboraf), Trametinib (Mekinist), and Dabrafenib (Tafinlar). Other examples of molecular targeted therapeutics targeting mutated oncogenes, include PLX27892 which targets mutant B-raf in melanoma.

Monoclonal antibody therapy is another targeted therapy in which the therapeutic agent is an antibody which specifically binds to a protein on the surface of the cancer cells. Examples include the anti-HER2/neu antibody trastuzumab (Herceptin) used in breast cancer, and the anti-CD20 antibody rituximab, used in a variety of B-cell malignancies. Other examples of monoclonal antibodies include Pembrolizumab (Keytruda), Alemtuzumab, Cetuximab, Panitumumab, Bevacizumab, and Ipilimumab (Yervoy).

Targeted therapy can also involve small peptides as “homing devices” which can bind to cell surface receptors or affected extracellular matrix surrounding the tumor. Radionuclides which are attached to these peptides (e.g. RGDs) eventually kill the cancer cell if the nuclide decays in the vicinity of the cell. Oligo- or multimers of these binding motifs can lead to enhanced tumor specificity and avidity.

Photodynamic therapy (PDT) is a ternary treatment for cancer involving a photosensitizer, tissue oxygen, and light (often using lasers). PDT can be used as treatment for basal cell carcinoma (BCC) or lung cancer. PDT can also be useful in removing traces of malignant tissue after surgical removal of large tumors. For example, iridium attached to albumin creates a photosensitized molecule that can penetrate cancer cells and, after being irradiated with light, destroy the cancer cells.

Targeted therapies also include treatments that include morpholino splice switching oligonucleotides, which induce ERG exon skipping in prostate cancer models, multitargeted kinase inhibitors that inhibit the PI3K pathway with other pathways including MEK and PIM, and inhibitors of NF-κB.

### Antibodies

In example embodiments, the gene expression product of the target genes (e.g., CDS2, ELMO2, ITGAV, AP1M1, FERMT2, CHMP4B, PTK2, ITGB5, PRKAR1A, ACTR1A, SRF, ZEB1, MTOR, DNM1L PSMB7, MOB4, TEAD1, DOCK5, ACTR3, BCAR1, UFM1, TIPARP, ILK, RSU1, WWTR1, NCKAP1, ATP6V1C1, MYH9, FOSL1, LIMS1, GNB1, EFR3A, ARHGEF7, WDR7, FERMT2, PKN2, CEMIP2, TEAD1, CTDNEP1, MARK3, VCL, CDC42, BCAR1, EGLN1, UFM1, GPX4, and CRK) are targeted using antibodies designed to bind the gene expression product of each target gene. The term “antibody” is used interchangeably with the term “immunoglobulin” herein, and includes intact antibodies, fragments of antibodies, e.g., Fab, F(ab′)2 fragments, and intact antibodies and fragments that have been mutated either in their constant and/or variable region (e.g., mutations to produce chimeric, partially humanized, or fully humanized antibodies, as well as to produce antibodies with a desired trait, e.g., enhanced binding and/or reduced FcR binding). The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)2, Fabc, Fd, dAb, V_{HH} and scFv and/or Fv fragments.

As used herein, a preparation of antibody protein having less than about 50% of non-antibody protein (also referred to herein as a “contaminating protein”), or of chemical precursors, is considered to be “substantially free.” 40%, 30%, 20%, 10% and more preferably 5% (by dry weight), of non-antibody protein, or of chemical precursors is considered to be substantially free. When the antibody protein or biologically active portion thereof is recombinantly produced, it is also preferably substantially free of culture medium, i.e., culture medium represents less than about 30%, preferably less than about 20%, more preferably less than about 10%, and most preferably less than about 5% of the volume or mass of the protein preparation.

The term “antigen-binding fragment” refers to a polypeptide fragment of an immunoglobulin or antibody that binds antigen or competes with intact antibody (i.e., with the intact antibody from which they were derived) for antigen binding (i.e., specific binding). As such these antibodies or fragments thereof are included in the scope of the disclosure, provided that the antibody or fragment binds specifically to a target molecule.

It is intended that the term “antibody” encompass any Ig class or any Ig subclass (e.g. the IgG1, IgG2, IgG3, and IgG4 subclasses of IgG) obtained from any source (e.g., humans and non-human primates, and in rodents, lagomorphs, caprines, bovines, equines, ovines, etc.).

The term “Ig class” or “immunoglobulin class”, as used herein, refers to the five classes of immunoglobulin that have been identified in humans and higher mammals, IgG, IgM, IgA, IgD, and IgE. The term “Ig subclass” refers to the two subclasses of IgM (H and L), three subclasses of IgA (IgA1, IgA2, and secretory IgA), and four subclasses of IgG (IgG1, IgG2, IgG3, and IgG4) that have been identified in humans and higher mammals. The antibodies can exist in monomeric or polymeric form; for example, IgM antibodies exist in pentameric form, and IgA antibodies exist in monomeric, dimeric or multimeric form.

The term “IgG subclass” refers to the four subclasses of immunoglobulin class IgG-IgG1, IgG2, IgG3, and IgG4 that have been identified in humans and higher mammals by the heavy chains of the immunoglobulins, V1-γ4, respectively. The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind antigen. The term “domain” refers to a globular region of a heavy or light chain polypeptide comprising peptide loops (e.g., comprising 3 to 4 peptide loops) stabilized, for example, by p pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions”. The “constant” domains of an antibody light chain are referred to interchangeably as “light chain constant regions”, “light chain constant domains”, “CL” regions or “CL” domains. The “constant” domains of an antibody heavy chain are referred to interchangeably as “heavy chain constant regions”, “heavy chain constant domains”, “CH” regions or “CH” domains). The “variable” domains of an antibody light chain are referred to interchangeably as “light chain variable regions”, “light chain variable domains”, “VL” regions or “VL” domains). The “variable” domains of an antibody heavy chain are referred to interchangeably as “heavy chain constant regions”, “heavy chain constant domains”, “VH” regions or “VH” domains).

The term “region” can also refer to a part or portion of an antibody chain or antibody chain domain (e.g., a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “framework regions” or “FRs”, as defined herein.

The term “conformation” refers to the tertiary structure of a protein or polypeptide (e.g., an antibody, antibody chain, domain or region thereof). For example, the phrase “light (or heavy) chain conformation” refers to the tertiary structure of a light (or heavy) chain variable region, and the phrase “antibody conformation” or “antibody fragment conformation” refers to the tertiary structure of an antibody or fragment thereof.

The term “antibody-like protein scaffolds” or “engineered protein scaffolds” broadly encompasses proteinaceous non-immunoglobulin specific-binding agents, typically obtained by combinatorial engineering (such as site-directed random mutagenesis in combination with phage display or other molecular selection techniques). Usually, such scaffolds are derived from robust and small soluble monomeric proteins (such as Kunitz inhibitors or lipocalins) or from a stably folded extra-membrane domain of a cell surface receptor (such as protein A, fibronectin or the ankyrin repeat).

Such scaffolds have been extensively reviewed in Binz et al. (Engineering novel binding proteins from nonimmunoglobulin domains. Nat Biotechnol 2005, 23:1257-1268), Gebauer and Skerra (Engineered protein scaffolds as next-generation antibody therapeutics. Curr Opin Chem Biol. 2009, 13:245-55), Gill and Damle (Biopharmaceutical drug discovery using novel protein scaffolds. Curr Opin Biotechnol 2006, 17:653-658), Skerra (Engineered protein scaffolds for molecular recognition. J Mol Recognit 2000, 13:167-187), and Skerra (Alternative non-antibody scaffolds for molecular recognition. Curr Opin Biotechnol 2007, 18:295-304), and include without limitation affibodies, based on the Z-domain of staphylococcal protein A, a three-helix bundle of 58 residues providing an interface on two of its alpha-helices (Nygren, Alternative binding proteins: Affibody binding proteins developed from a small three-helix bundle scaffold. FEBS J 2008, 275:2668-2676); engineered Kunitz domains based on a small (ca. 58 residues) and robust, disulphide-crosslinked serine protease inhibitor, typically of human origin (e.g. LACI-D1), which can be engineered for different protease specificities (Nixon and Wood, Engineered protein inhibitors of proteases. Curr Opin Drug Discov Dev 2006, 9:261-268); monobodies or adnectins based on the 10th extracellular domain of human fibronectin III (10Fn3), which adopts an Ig-like beta-sandwich fold (94 residues) with 2-3 exposed loops, but lacks the central disulphide bridge (Koide and Koide, Monobodies: antibody mimics based on the scaffold of the fibronectin type III domain. Methods Mol Biol 2007, 352:95-109); anticalins derived from the lipocalins, a diverse family of eight-stranded beta-barrel proteins (ca. 180 residues) that naturally form binding sites for small ligands by means of four structurally variable loops at the open end, which are abundant in humans, insects, and many other organisms (Skerra, Alternative binding proteins: Anticalins—harnessing the structural plasticity of the lipocalin ligand pocket to engineer novel binding activities. FEBS J 2008, 275:2677-2683); DARPins, designed ankyrin repeat domains (166 residues), which provide a rigid interface arising from typically three repeated beta-turns (Stumpp et al., DARPins: a new generation of protein therapeutics. Drug Discov Today 2008, 13:695-701); avimers (multimerized LDLR-A module) (Silverman et al., Multivalent avimer proteins evolved by exon shuffling of a family of human receptor domains. Nat Biotechnol 2005, 23:1556-1561); and cysteine-rich knottin peptides (Kolmar, Alternative binding proteins: biological activity and therapeutic potential of cystine-knot miniproteins. FEBS J 2008, 275:2684-2690).

“Specific binding” of an antibody means that the antibody exhibits appreciable affinity for a particular antigen or epitope and, generally, does not exhibit significant cross reactivity. “Appreciable” binding includes binding with an affinity of at least 25 μM. Antibodies with affinities greater than 1×10⁷ M⁻¹ (or a dissociation coefficient of 1 μM or less or a dissociation coefficient of 1 nm or less) typically bind with correspondingly greater specificity. Values intermediate of those set forth herein are also intended to be within the scope of the present disclosure and antibodies of the disclosure bind with a range of affinities, for example, 100 nM or less, 75 nM or less, 50 nM or less, 25 nM or less, for example 10 nM or less, 5 nM or less, 1 nM or less, or in embodiments 500 pM or less, 100 pM or less, 50 pM or less or 25 pM or less. An antibody that “does not exhibit significant cross reactivity” is one that will not appreciably bind to an entity other than its target (e.g., a different epitope or a different molecule). For example, an antibody that specifically binds to a target molecule will appreciably bind the target molecule but will not significantly react with non-target molecules or peptides. An antibody specific for a particular epitope will, for example, not significantly cross-react with remote epitopes on the same protein or peptide. Specific binding can be determined according to any art-recognized means for determining such binding. Preferably, specific binding is determined according to Scatchard analysis and/or competitive binding assays.

As used herein, the term “affinity” refers to the strength of the binding of a single antigen-combining site with an antigenic determinant. Affinity depends on the closeness of stereochemical fit between antibody combining sites and antigen determinants, on the size of the area of contact between them, on the distribution of charged and hydrophobic groups, etc. Antibody affinity can be measured by equilibrium dialysis or by the kinetic BIACORE™ method. The dissociation constant, Kd, and the association constant, Ka, are quantitative measures of affinity.

As used herein, the term “monoclonal antibody” refers to an antibody derived from a clonal population of antibody-producing cells (e.g., B lymphocytes or B cells) which is homogeneous in structure and antigen specificity. The term “polyclonal antibody” refers to a plurality of antibodies originating from different clonal populations of antibody-producing cells which are heterogeneous in their structure and epitope specificity but which recognize a common antigen. Monoclonal and polyclonal antibodies may exist within bodily fluids, as crude preparations, or may be purified, as described herein.

The term “binding portion” of an antibody (or “antibody portion”) includes one or more complete domains, e.g., a pair of complete domains, as well as fragments of an antibody that retain the ability to specifically bind to a target molecule. It has been shown that the binding function of an antibody can be performed by fragments of a full-length antibody. Binding fragments are produced by recombinant DNA techniques, or by enzymatic or chemical cleavage of intact immunoglobulins. Binding fragments include Fab, Fab′, F(ab′)2, Fabc, Fd, dAb, Fv, single chains, single-chain antibodies, e.g., scFv, and single domain antibodies.

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, FR residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin.

Examples of portions of antibodies or epitope-binding proteins encompassed by the present definition include: (i) the Fab fragment, having V_{L}, C_{L}, V_{H} and C_{H}1 domains; (ii) the Fab′ fragment, which is a Fab fragment having one or more cysteine residues at the C-terminus of the C_{H}1 domain; (iii) the Fd fragment having V_{H} and C_{H}1 domains; (iv) the Fd′ fragment having V_{H} and C_{H}1 domains and one or more cysteine residues at the C-terminus of the CHI domain; (v) the Fv fragment having the V_{L} and V_{H} domains of a single arm of an antibody; (vi) the dAb fragment (Ward et al., 341 Nature 544 (1989)) which consists of a V_{H} domain or a V_{L} domain that binds antigen; (vii) isolated CDR regions or isolated CDR regions presented in a functional framework; (viii) F(ab′)₂ fragments which are bivalent fragments including two Fab′ fragments linked by a disulphide bridge at the hinge region; (ix) single chain antibody molecules (e.g., single chain Fv; scFv) (Bird et al., 242 Science 423 (1988); and Huston et al., 85 PNAS 5879 (1988)); (x) “diabodies” with two antigen binding sites, comprising a heavy chain variable domain (V_{H}) connected to a light chain variable domain (V_{L}) in the same polypeptide chain (see, e.g., EP 404,097; WO 93/11161; Hollinger et al., 90 PNAS 6444 (1993)); (xi) “linear antibodies” comprising a pair of tandem Fd segments (V_{H}-Cₕ1-V_{H}-Cₕ1) which, together with complementary light chain polypeptides, form a pair of antigen binding regions (Zapata et al., Protein Eng. 8(10):1057-62 (1995); and U.S. Pat. No. 5,641,870).

As used herein, a “blocking” antibody or an antibody “antagonist” is one which inhibits or reduces biological activity of the antigen(s) it binds. In certain embodiments, the blocking antibodies or antagonist antibodies or portions thereof described herein completely inhibit the biological activity of the antigen(s).

Antibodies may act as agonists or antagonists of the recognized polypeptides. For example, the present disclosure includes antibodies which disrupt receptor/ligand interactions either partially or fully. The disclosure features both receptor-specific antibodies and ligand-specific antibodies. The disclosure also features receptor-specific antibodies which do not prevent ligand binding but prevent receptor activation. Receptor activation (i.e., signaling) may be determined by techniques described herein or otherwise known in the art. For example, receptor activation can be determined by detecting the phosphorylation (e.g., tyrosine or serine/threonine) of the receptor or of one of its down-stream substrates by immunoprecipitation followed by western blot analysis. In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

In certain embodiments, receptors are targeted with antibodies that block ligand binding. The disclosure also features receptor-specific antibodies which both prevent ligand binding and receptor activation as well as antibodies that recognize the receptor-ligand complex. Likewise, encompassed by the disclosure are neutralizing antibodies which bind the ligand and prevent binding of the ligand to the receptor, as well as antibodies which bind the ligand, thereby preventing receptor activation, but do not prevent the ligand from binding the receptor. Further included in the disclosure are antibodies which activate the receptor. These antibodies may act as receptor agonists, i.e., potentiate or activate either all or a subset of the biological activities of the ligand-mediated receptor activation, for example, by inducing dimerization of the receptor. The antibodies may be specified as agonists, antagonists or inverse agonists for biological activities comprising the specific biological activities of the peptides disclosed herein. The antibody agonists and antagonists can be made using methods known in the art. See, e.g., PCT publication WO 96/40281; U.S. Pat. No. 5,811,097; Deng et al., Blood 92(6):1981-1988 (1998); Chen et al., Cancer Res. 58(16):3668-3678 (1998); Harrop et al., J. Immunol. 161(4):1786-1794 (1998); Zhu et al., Cancer Res. 58(15):3209-3214 (1998); Yoon et al., J. Immunol. 160(7):3170-3179 (1998); Prat et al., J. Cell. Sci. III (Pt2):237-247 (1998); Pitard et al., J. Immunol. Methods 205(2):177-190 (1997); Liautard et al., Cytokine 9(4):233-241 (1997); Carlson et al., J. Biol. Chem. 272(17):11295-11301 (1997); Taryman et al., Neuron 14(4):755-762 (1995); Muller et al., Structure 6(9):1153-1167 (1998); Bartunek et al., Cytokine 8(1):14-20 (1996).

The antibodies as defined for the present disclosure include derivatives that are modified, i.e., by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

Simple binding assays can be used to screen for or detect agents that bind to a target protein, or disrupt the interaction between proteins (e.g., a receptor and a ligand). Because certain targets of the present disclosure are transmembrane proteins, assays that use the soluble forms of these proteins rather than full-length protein can be used, in some embodiments. Soluble forms include, for example, those lacking the transmembrane domain and/or those comprising the IgV domain or fragments thereof which retain their ability to bind their cognate binding partners. Further, agents that inhibit or enhance protein interactions for use in the compositions and methods described herein, can include recombinant peptido-mimetics.

Detection methods useful in screening assays include antibody-based methods, detection of a reporter moiety, detection of cytokines as described herein, and detection of a gene signature as described herein.

Another variation of assays to determine binding of a receptor protein to a ligand protein is through the use of affinity biosensor methods. Such methods may be based on the piezoelectric effect, electrochemistry, or optical methods, such as ellipsometry, optical wave guidance, and surface plasmon resonance (SPR).

### Aptamers

In certain embodiments, the gene expression product of the target genes (e.g., CDS2, ELMO2, ITGAV, AP1M1, FERMT2, CHMP4B, PTK2, ITGB5, PRKAR1A, ACTR1A, SRF, ZEB1, MTOR, DNM1L PSMB7, MOB4, TEAD1, DOCK5, ACTR3, BCAR1, UFM1, TIPARP, ILK, RSU1, WWTR1, NCKAP1, ATP6V1C1, MYH9, FOSL1, LIMS1, GNB1, EFR3A, ARHGEF7, WDR7, FERMT2, PKN2, CEMIP2, TEAD1, CTDNEP1, MARK3, VCL, CDC42, BCAR1, EGLN1, UFM1, GPX4, and CRK) are targeted by aptamers designed to bind to one of the gene expression products of the target gene. Nucleic acid aptamers are nucleic acid species that have been engineered through repeated rounds of in vitro selection or equivalently, SELEX (systematic evolution of ligands by exponential enrichment) to bind to various molecular targets such as small molecules, proteins, nucleic acids, cells, tissues and organisms. Nucleic acid aptamers have specific binding affinity to molecules through interactions other than classic Watson-Crick base pairing. Aptamers are useful in biotechnological and therapeutic applications as they offer molecular recognition properties similar to antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications. In certain embodiments, RNA aptamers may be expressed from a DNA construct. In other embodiments, a nucleic acid aptamer may be linked to another polynucleotide sequence. The polynucleotide sequence may be a double stranded DNA polynucleotide sequence. The aptamer may be covalently linked to one strand of the polynucleotide sequence. The aptamer may be ligated to the polynucleotide sequence. The polynucleotide sequence may be configured, such that the polynucleotide sequence may be linked to a solid support or ligated to another polynucleotide sequence.

Aptamers, like peptides generated by phage display or monoclonal antibodies (“mAbs”), are capable of specifically binding to selected targets and modulating the target's activity, e.g., through binding, aptamers may block their target's ability to function. A typical aptamer is 10-15 kDa in size (30-45 nucleotides), binds its target with sub-nanomolar affinity, and discriminates against closely related targets (e.g., aptamers will typically not bind other proteins from the same gene family). Structural studies have shown that aptamers are capable of using the same types of binding interactions (e.g., hydrogen bonding, electrostatic complementarity, hydrophobic contacts, steric exclusion) that drives affinity and specificity in antibody-antigen complexes.

Aptamers have a number of desirable characteristics for use in research and as therapeutics and diagnostics including high specificity and affinity, biological efficacy, and excellent pharmacokinetic properties. In addition, they offer specific competitive advantages over antibodies and other protein biologics. Aptamers are chemically synthesized and are readily scaled as needed to meet production demand for research, diagnostic or therapeutic applications. Aptamers are chemically robust. They are intrinsically adapted to regain activity following exposure to factors such as heat and denaturants and can be stored for extended periods (>1 yr) at room temperature as lyophilized powders. Not being bound by a theory, aptamers bound to a solid support or beads may be stored for extended periods.

Oligonucleotides in their phosphodiester form may be quickly degraded by intracellular and extracellular enzymes such as endonucleases and exonucleases. Aptamers can include modified nucleotides conferring improved characteristics on the ligand, such as improved in vivo stability or improved delivery characteristics. Examples of such modifications include chemical substitutions at the ribose and/or phosphate and/or base positions. SELEX identified nucleic acid ligands containing modified nucleotides are described, e.g., in U.S. Pat. No. 5,660,985, which describes oligonucleotides containing nucleotide derivatives chemically modified at the 2′ position of ribose, 5 position of pyrimidines, and 8 position of purines, U.S. Pat. No. 5,756,703 which describes oligonucleotides containing various 2′-modified pyrimidines, and U.S. Pat. No. 5,580,737 which describes highly specific nucleic acid ligands containing one or more nucleotides modified with 2′-amino (2′-NH₂), 2′-fluoro (2′-F), and/or 2′-0-methyl (2′-OMe) substituents. Modifications of aptamers may also include modifications at exocyclic amines, substitution of 4-thiouridine, substitution of 5-bromo or 5-iodo-uracil; backbone modifications, phosphorothioate or allyl phosphate modifications, methylations, and unusual base-pairing combinations such as the isobases isocytidine and isoguanosine. Modifications can also include 3′ and 5′ modifications such as capping. As used herein, the term phosphorothioate encompasses one or more non-bridging oxygen atoms in a phosphodiester bond replaced by one or more sulfur atoms. In further embodiments, the oligonucleotides comprise modified sugar groups, for example, one or more of the hydroxyl groups is replaced with halogen, aliphatic groups, or functionalized as ethers or amines. In one embodiment, the 2′-position of the furanose residue is substituted by any of an O-methyl, O-alkyl, O-allyl, S-alkyl, S-allyl, or halo group. Methods of synthesis of 2′-modified sugars are described, e.g., in Sproat, et al., Nucl. Acid Res. 19:733-738 (1991); Cotten, et al, Nucl. Acid Res. 19:2629-2635 (1991); and Hobbs, et al, Biochemistry 12:5138-5145 (1973). Other modifications are known to one of ordinary skill in the art. In certain embodiments, aptamers include aptamers with improved off-rates as described in International Patent Publication No. WO 2009012418, “Method for generating aptamers with improved off-rates,” incorporated herein by reference in its entirety. In certain embodiments aptamers are chosen from a library of aptamers. Such libraries include, but are not limited to those described in Rohloff et al., “Nucleic Acid Ligands With Protein-like Side Chains: Modified Aptamers and Their Use as Diagnostic and Therapeutic Agents,” Molecular Therapy Nucleic Acids (2014) 3, e201. Aptamers are also commercially available (see, e.g., SomaLogic, Inc., Boulder, Colorado). In certain embodiments, the present disclosure may utilize any aptamer containing any modification as described herein.

### Genetic Modifying Agents

In certain embodiments, tumor cells having a mesenchymal state are targeted with a genetic modifying agent configured to modify one or more of the target genes (e.g., CDS2, ELMO2, ITGAV, AP1M1, FERMT2, CHMP4B, PTK2, ITGB5, PRKAR1A, ACTR1A, SRF, ZEB1, MTOR, DNM1L PSMB7, MOB4, TEAD1, DOCK5, ACTR3, BCAR1, UFM1, TIPARP, ILK, RSU1, WWTR1, NCKAP1, ATP6V1C1, MYH9, FOSL1, LIMS1, GNB1, EFR3A, ARHGEF7, WDR7, FERMT2, PKN2, CEMIP2, TEAD1, CTDNEP1, MARK3, VCL, CDC42, BCAR1, EGLN1, UFM1, GPX4, and CRK; and any combination thereof). The genetic modifying agent may comprise a programmable nuclease, such as, a CRISPR system, a zinc finger nuclease system, a TALEN, or a meganuclease. In some embodiments, a polynucleotide of the present disclosure described elsewhere herein can be modified using a genetic modifying agent. In example embodiments, the genetic modifying agent is administered using a vector, such as a viral vector or liposome. In example embodiments, the genetic modifying agent is targeted to tumor cells (see, e.g., Montaño-Samaniego M, Bravo-Estupiñan DM, Mendez-Guerrero O, Alarcón-Hernández E, Ibáñez-Hernández M. Strategies for Targeting Gene Therapy in Cancer Cells With Tumor-Specific Promoters. Front Oncol. 2020; 10:605380; and Jafari M, Kadkhodazadeh M, Shapourabadi M B, et al. Immunovirotherapy: The role of antibody based therapeutics combination with oncolytic viruses. Front Immunol. 2022; 13:1012806). In example embodiments, the genetic modifying agent is administered directly to a tumor.

### CRISPR-Cas

In one example embodiment, the genetic modifying agent is a CRISPR-Cas system. CRISPR-Cas systems comprise a Cas polypeptide and a guide sequence, wherein the guide sequence is capable of forming a CRISPR-Cas complex with the Cas polypeptide and directing site-specific binding of the CRISPR-Cas sequence to a target sequence in one or more of the target genes. The Cas polypeptide may induce a double- or single-stranded break at a designated site in the target sequence. The site of CRISPR-Cas cleavage, for most CRISPR-Cas systems, is dictated by distance from a protospacer-adjacent motif (PAM), discussed in further detail below. Accordingly, a guide sequence may be selected to direct the CRISPR-Cas system to a desired target site at or near the one or more target genes. Additionally, CRISPR systems can be used in vivo (see, e.g., Chen H, Shi M, Gilam A, et al. Hemophilia A ameliorated in mice by CRISPR-based in vivo genome editing of human Factor VIII. Sci Rep. 2019; 9(1):16838; Hana S, Peterson M, McLaughlin H, et al. Highly efficient neuronal gene knockout in vivo by CRISPR-Cas9 via neonatal intracerebroventricular injection of AAV in mice. Gene Ther. 2021; 28(10-11):646-658; and Rosenblum D, Gutkin A, Kedmi R, et al. CRISPR-Cas9 genome editing using targeted lipid nanoparticles for cancer therapy. Sci Adv. 2020; 6(47):eabc9450).

In general, a CRISPR-Cas or CRISPR system as used in herein and in documents, such as International Patent Publication No. WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g., Shmakov et al. (2015) “Discovery and Functional Characterization of Diverse Class 2 CRISPR-Cas Systems”, Molecular Cell, DOI: dx.doi.org/10.1016/j.molcel.2015.10.008.

CRISPR-Cas systems can generally fall into two classes based on their architectures of their effector molecules, which are each further subdivided by type and subtype. The two class are Class 1 and Class 2. Class 1 CRISPR-Cas systems have effector modules composed of multiple Cas proteins, some of which form crRNA-binding complexes, while Class 2 CRISPR-Cas systems include a single, multi-domain crRNA-binding protein.

In some embodiments, the CRISPR-Cas system that can be used to modify a polynucleotide of the present disclosure can be a Class 1 CRISPR-Cas system. In some embodiments, the CRISPR-Cas system that can be used to modify a polynucleotide of the present disclosure can be a Class 2 CRISPR-Cas system.

### Class 1 CRISPR-Cas Systems

In some embodiments, the CRISPR-Cas system that can be used to modify a polynucleotide of the present disclosure can be a Class 1 CRISPR-Cas system. Class 1 CRISPR-Cas systems are divided into types I, II, and IV. Makarova et al. 2020. Nat. Rev. 18: 67-83, particularly as described in FIG. 1. Type I CRISPR-Cas systems are divided into 9 subtypes (I-A, I-B, I-C, I-D, I-E, I-F1, I-F2, I-F3, and IG). Makarova et al., 2020. Class 1, Type I CRISPR-Cas systems can contain a Cas3 protein that can have helicase activity. Type III CRISPR-Cas systems are divided into 6 subtypes (III-A, III-B, III-C, III-D, III-E, and III-F). Type III CRISPR-Cas systems can contain a Cas10 that can include an RNA recognition motif called Palm and a cyclase domain that can cleave polynucleotides. Makarova et al., 2020. Type IV CRISPR-Cas systems are divided into 3 subtypes. (IV-A, IV-B, and IV-C). Makarova et al., 2020. Class 1 systems also include CRISPR-Cas variants, including Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems. Peters et al., PNAS 114 (35) (2017); DOI: 10.1073/pnas.1709035114; see also, Makarova et al. 2018. The CRISPR Journal, v. 1, n5, FIG. 5.

The Class 1 systems typically comprise a multi-protein effector complex, which can, in some embodiments, include ancillary proteins, such as one or more proteins in a complex referred to as a CRISPR-associated complex for antiviral defense (Cascade), one or more adaptation proteins (e.g., Cas1, Cas2, RNA nuclease), and/or one or more accessory proteins (e.g., Cas 4, DNA nuclease), CRISPR associated Rossman fold (CARF) domain containing proteins, and/or RNA transcriptase.

The backbone of the Class 1 CRISPR-Cas system effector complexes can be formed by RNA recognition motif domain-containing protein(s) of the repeat-associated mysterious proteins (RAMPs) family subunits (e.g., Cas 5, Cas6, and/or Cas7). RAMP proteins are characterized by having one or more RNA recognition motif domains. In some embodiments, multiple copies of RAMPs can be present. In some embodiments, the Class I CRISPR-Cas system can include 1, 2, 3, 4, 5, 6, 7, 8, 9, 10, 11, 12 or more Cas5, Cas6, and/or Cas 7 proteins. In some embodiments, the Cas6 protein is an RNAse, which can be responsible for pre-crRNA processing. When present in a Class 1 CRISPR-Cas system, Cas6 can be optionally physically associated with the effector complex.

Class 1 CRISPR-Cas system effector complexes can, in some embodiments, also include a large subunit. The large subunit can be composed of or include a Cas8 and/or Cas10 protein. See, e.g., FIGS. 1 and 2. Koonin E V, Makarova K S. 2019. Phil. Trans. R. Soc. B 374: 20180087, DOI: 10.1098/rstb.2018.0087 and Makarova et al. 2020.

Class 1 CRISPR-Cas system effector complexes can, in some embodiments, include a small subunit (for example, Cas11). See, e.g., FIGS. 1 and 2. Koonin E V, Makarova K S. 2019 Origins and Evolution of CRISPR-Cas systems. Phil. Trans. R. Soc. B 374: 20180087, DOI: 10.1098/rstb.2018.0087.

In some embodiments, the Class 1 CRISPR-Cas system can be a Type I CRISPR-Cas system. In some embodiments, the Type I CRISPR-Cas system can be a subtype I-A CRISPR-Cas system. In some embodiments, the Type I CRISPR-Cas system can be a subtype I-B CRISPR-Cas system. In some embodiments, the Type I CRISPR-Cas system can be a subtype I-C CRISPR-Cas system. In some embodiments, the Type I CRISPR-Cas system can be a subtype I-D CRISPR-Cas system. In some embodiments, the Type I CRISPR-Cas system can be a subtype I-E CRISPR-Cas system. In some embodiments, the Type I CRISPR-Cas system can be a subtype I-F1 CRISPR-Cas system. In some embodiments, the Type I CRISPR-Cas system can be a subtype I-F2 CRISPR-Cas system. In some embodiments, the Type I CRISPR-Cas system can be a subtype I-F3 CRISPR-Cas system. In some embodiments, the Type I CRISPR-Cas system can be a subtype I-G CRISPR-Cas system. In some embodiments, the Type I CRISPR-Cas system can be a CRISPR Cas variant, such as a Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems as previously described.

In some embodiments, the Class 1 CRISPR-Cas system can be a Type III CRISPR-Cas system. In some embodiments, the Type III CRISPR-Cas system can be a subtype III-A CRISPR-Cas system. In some embodiments, the Type III CRISPR-Cas system can be a subtype III-B CRISPR-Cas system. In some embodiments, the Type III CRISPR-Cas system can be a subtype III-C CRISPR-Cas system. In some embodiments, the Type III CRISPR-Cas system can be a subtype III-D CRISPR-Cas system. In some embodiments, the Type III CRISPR-Cas system can be a subtype III-E CRISPR-Cas system. In some embodiments, the Type III CRISPR-Cas system can be a subtype III-F CRISPR-Cas system.

In some embodiments, the Class 1 CRISPR-Cas system can be a Type IV CRISPR-Cas-system. In some embodiments, the Type IV CRISPR-Cas system can be a subtype IV-A CRISPR-Cas system. In some embodiments, the Type IV CRISPR-Cas system can be a subtype IV-B CRISPR-Cas system. In some embodiments, the Type IV CRISPR-Cas system can be a subtype IV-C CRISPR-Cas system.

The effector complex of a Class 1 CRISPR-Cas system can, in some embodiments, include a Cas3 protein that is optionally fused to a Cas2 protein, a Cas4, a Cas5, a Cas6, a Cas7, a Cas8, a Cas10, a Cas11, or a combination thereof. In some embodiments, the effector complex of a Class 1 CRISPR-Cas system can have multiple copies, such as 1, 2, 3, 4, 5, 6, 7, 8, 9, 10, 11, 12, 13, or 14, of any one or more Cas proteins.

### Class 2 CRISPR-Cas Systems

The compositions, systems, and methods described in greater detail elsewhere herein can be designed and adapted for use with Class 2 CRISPR-Cas systems. Thus, in some embodiments, the CRISPR-Cas system is a Class 2 CRISPR-Cas system. Class 2 systems are distinguished from Class 1 systems in that they have a single, large, multi-domain effector protein. In certain example embodiments, the Class 2 system can be a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. Each type of Class 2 system is further divided into subtypes. See Markova et al. 2020, particularly at Figure. 2. Class 2, Type II systems can be divided into 4 subtypes: II-A, II-B, 1-C1, and II-C2. Class 2, Type V systems can be divided into 17 subtypes: V-A, V-B1, V-B2, V-C, V-D, V-E, V-F1, V-F1(V-U3), V-F2, V-F3, V-G, V-H, V-I, V-K (V-U5), V-U1, V-U2, and V-U4. Class 2, Type IV systems can be divided into 5 subtypes: VI-A, VI-B1, VI-B2, VI-C, and VI-D.

The distinguishing feature of these types is that their effector complexes consist of a single, large, multi-domain protein. Type V systems differ from Type II effectors (e.g., Cas9), which contain two nuclear domains that are each responsible for the cleavage of one strand of the target DNA, with the HNH nuclease inserted inside the Ruv-C like nuclease domain sequence. The Type V systems (e.g., Cas12) only contain a RuvC-like nuclease domain that cleaves both strands. Type VI (Cas13) are unrelated to the effectors of Type II and V systems and contain two HEPN domains and target RNA. Cas13 proteins also display collateral activity that is triggered by target recognition. Some Type V systems have also been found to possess this collateral activity with two single-stranded DNA in in vitro contexts.

In some embodiments, the Class 2 system is a Type II system. In some embodiments, the Type II CRISPR-Cas system is a II-A CRISPR-Cas system. In some embodiments, the Type II CRISPR-Cas system is a II-B CRISPR-Cas system. In some embodiments, the Type I CRISPR-Cas system is a II-C1 CRISPR-Cas system. In some embodiments, the Type II CRISPR-Cas system is a II-C2 CRISPR-Cas system. In some embodiments, the Type II system is a Cas9 system. In some embodiments, the Type II system includes a Cas9.

In some embodiments, the Class 2 system is a Type V system. In some embodiments, the Type V CRISPR-Cas system is a V-A CRISPR-Cas system. In some embodiments, the Type V CRISPR-Cas system is a V-B1 CRISPR-Cas system. In some embodiments, the Type V CRISPR-Cas system is a V-B2 CRISPR-Cas system. In some embodiments, the Type V CRISPR-Cas system is a V-C CRISPR-Cas system. In some embodiments, the Type V CRISPR-Cas system is a V-D CRISPR-Cas system. In some embodiments, the Type V CRISPR-Cas system is a V-E CRISPR-Cas system. In some embodiments, the Type V CRISPR-Cas system is a V-F1 CRISPR-Cas system. In some embodiments, the Type V CRISPR-Cas system is a V-F1 (V-U3) CRISPR-Cas system. In some embodiments, the Type V CRISPR-Cas system is a V-F2 CRISPR-Cas system. In some embodiments, the Type V CRISPR-Cas system is a V-F3 CRISPR-Cas system. In some embodiments, the Type V CRISPR-Cas system is a V-G CRISPR-Cas system. In some embodiments, the Type V CRISPR-Cas system is a V-H CRISPR-Cas system. In some embodiments, the Type V CRISPR-Cas system is a V-I CRISPR-Cas system. In some embodiments, the Type V CRISPR-Cas system is a V-K (V-U5) CRISPR-Cas system. In some embodiments, the Type V CRISPR-Cas system is a V-U1 CRISPR-Cas system. In some embodiments, the Type V CRISPR-Cas system is a V-U2 CRISPR-Cas system. In some embodiments, the Type V CRISPR-Cas system is a V-U4 CRISPR-Cas system. In some embodiments, the Type V CRISPR-Cas system includes a Cas12a (Cpf1), Cas12b (C2c1), Cas12c (C2c3), Cas12d (CasY), Cas12e (CasX), Cas14, and/or CasΦ.

In some embodiments the Class 2 system is a Type VI system. In some embodiments, the Type VI CRISPR-Cas system is a VI-A CRISPR-Cas system. In some embodiments, the Type VI CRISPR-Cas system is a VI-B1 CRISPR-Cas system. In some embodiments, the Type VI CRISPR-Cas system is a VI-B2 CRISPR-Cas system. In some embodiments, the Type VI CRISPR-Cas system is a VI-C CRISPR-Cas system. In some embodiments, the Type VI CRISPR-Cas system is a VI-D CRISPR-Cas system. In some embodiments, the Type VI CRISPR-Cas system includes a Cas13a (C2c2), Cas13b (Group 29/30), Cas13c, and/or Cas13d.

### Guide Molecules

The following include general design principles that may be applied to the guide molecule. The terms guide molecule, guide sequence and guide polynucleotide refer to polynucleotides capable of guiding Cas to a target genomic locus and are used interchangeably as in foregoing cited documents such as International Patent Publication No. WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. The guide molecule can be a polynucleotide.

The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay (Qui et al. 2004. BioTechniques. 36(4)702-707). Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible and will occur to those skilled in the art.

In some embodiments, the guide molecule is an RNA. The guide molecule(s) (also referred to interchangeably herein as guide polynucleotide and guide sequence) that are included in the CRISPR-Cas or Cas based system can be any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, CA), SOAP (available at soap.genomics.org.cn), and Maq (available at maq.sourceforge.net).

A guide sequence, and hence a nucleic acid-targeting guide, may be selected to target any target nucleic acid sequence. The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (1ncRNA), and small cytoplasmatic RNA (scRNA). In some preferred embodiments, the target sequence may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

In some embodiments, a nucleic acid-targeting guide is selected to reduce the degree secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and PA Carr and GM Church, 2009, Nature Biotechnology 27(12): 1151-62).

In one example embodiment, a guide RNA or crRNA may comprise, consist essentially of, or consist of a direct repeat (DR) sequence and a guide sequence or spacer sequence. In another example embodiment, the guide RNA or crRNA may comprise, consist essentially of, or consist of a direct repeat sequence fused or linked to a guide sequence or spacer sequence. In another example embodiment, the direct repeat sequence may be located upstream (i.e., 5′) from the guide sequence or spacer sequence. In other embodiments, the direct repeat sequence may be located downstream (i.e., 3′) from the guide sequence or spacer sequence.

In one example embodiment, the crRNA comprises a stem loop, preferably a single stem loop. In one example embodiment, the direct repeat sequence forms a stem loop, preferably a single stem loop.

In one example embodiment, the spacer length of the guide RNA is from 15 to 35 nt. In another example embodiment, the spacer length of the guide RNA is at least 15 nucleotides. In another example embodiment, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and crRNA sequence are contained within a single transcript, such that hybridization between the two produces a transcript having a secondary structure, such as a hairpin.

In general, degree of complementarity is with reference to the optimal alignment of the spacer sequence and tracr sequence, along the length of the shorter of the two sequences. Optimal alignment may be determined by any suitable alignment algorithm and may further account for secondary structures, such as self-complementarity within either the spacer sequence or tracr sequence. In some embodiments, the degree of complementarity between the tracr sequence and spacer sequence along the length of the shorter of the two when optimally aligned is about or more than about 25%, 30%, 40%, 50%, 60%, 70%, 80%, 90%, 95%, 97.5%, 99%, or higher.

In some embodiments, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and tracr RNA can be 30 or 50 nucleotides in length. In some embodiments, the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it being advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In some embodiments according to the disclosure, the guide RNA (capable of guiding Cas to a target locus) may comprise (1) a guide sequence capable of hybridizing to a genomic target locus in the eukaryotic cell; (2) a tracr sequence; and (3) a tracr mate sequence. All of (1) to (3) may reside in a single RNA, i.e., an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence. Where the tracr RNA is on a different RNA than the RNA containing the guide and tracr sequence, the length of each RNA may be optimized to be shortened from their respective native lengths, and each may be independently chemically modified to protect from degradation by cellular RNase or otherwise increase stability.

Many modifications to guide sequences are known in the art and are further contemplated within the context of this disclosure. Various modifications may be used to increase the specificity of binding to the target sequence and/or increase the activity of the Cas protein and/or reduce off-target effects. Example guide sequence modifications are described in International Patent Application No. PCT US2019/045582, specifically paragraphs [0178]-[0333]. which is incorporated herein by reference.

### Target Sequences, PAMs, and PFSs

In the context of formation of a CRISPR complex, “target sequence” refers to a sequence to which a guide sequence is designed to have complementarity, where hybridization between a target sequence and a guide sequence promotes the formation of a CRISPR complex. In other words, the target polynucleotide can be a polynucleotide or a part of a polynucleotide to which a part of the guide sequence is designed to have complementarity with and to which the effector function mediated by the complex comprising the CRISPR effector protein and a guide molecule is to be directed. In some embodiments, a target sequence is located in the nucleus or cytoplasm of a cell.

PAM elements are sequences that can be recognized and bound by Cas proteins. Cas proteins/effector complexes can then unwind the dsDNA at a position adjacent to the PAM element. It will be appreciated that Cas proteins and systems target RNA do not require PAM sequences (Marraffini et al. 2010. Nature. 463:568-571). Instead, many rely on PFSs, which are discussed elsewhere herein. In one example embodiment, the target sequence should be associated with a PAM (protospacer adjacent motif) or PFS (protospacer flanking sequence or site), that is, a short sequence recognized by the CRISPR complex. Depending on the nature of the CRISPR-Cas protein, the target sequence should be selected, such that its complementary sequence in the DNA duplex (also referred to herein as the non-target sequence) is upstream or downstream of the PAM. In the embodiments, the complementary sequence of the target sequence is downstream or 3′ of the PAM or upstream or 5′ of the PAM. The precise sequence and length requirements for the PAM differ depending on the Cas protein used, but PAMs are typically 2-5 base pair sequences adjacent the protospacer (that is, the target sequence). Examples of the natural PAM sequences for different Cas proteins are provided herein below and the skilled person will be able to identify further PAM sequences for use with a given Cas protein.

The ability to recognize different PAM sequences depends on the Cas polypeptide(s) included in the system. See e.g., Gleditzsch et al. 2019. RNA Biology. 16(4):504-517. Table A (from Gleditzsch et al. 2019) below shows several Cas polypeptides and the PAM sequence they recognize.

TABLE A
Example PAM Sequences

| Cas Protein | PAM Sequence |
|---|---|
| SpCas9 | NGG/NRG |
| SaCas9 | NGRRT or NGRRN |
| NmeCas9 | NNNNGATT |
| CjCas9 | NNNNRYAC |
| StCas9 | NNAGAAW |
| Cas12a (Cpf1) (including | TTTV |
| LbCpf1 and AsCpf1) | |
| Cas12b (C2c1) | TTT, TTA, and TTC |
| Cas12c (C2c3) | TA |
| Cas12d (CasY) | TA |
| Cas12e (CasX) | 5′-TTCN-3′ |
| Cas1 | 5′-CTT-3′ |
| Cas8e | 5′-ATG-3′ |
| Type I-A | 5′-CCN-3′ |
| Type I-B | TTC, ACT, TAA, TAT, |
| | TAG, and CAC |
| Type I-C | NTTC |
| Type I-E | 5′-AAG-3′ |
| Type I-F | GG |

In a preferred embodiment, the CRISPR effector protein may recognize a 3′ PAM. In one example embodiment, the CRISPR effector protein may recognize a 3′ PAM which is 5′H, wherein H is A, C or U.

Further, engineering of the PAM Interacting (PI) domain on the Cas protein may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481-5. doi: 10.1038/nature14592. As further detailed herein, the skilled person will understand that Cas13 proteins may be modified analogously. Gao et al, “Engineered Cpf1 Enzymes with Altered PAM Specificities,” bioRxiv 091611; doi: http://dx.doi.org/10.1101/091611 (Dec. 4, 2016). Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.

PAM sequences can be identified in a polynucleotide using an appropriate design tool, which are commercially available as well as online. Such freely available tools include, but are not limited to, CRISPRFinder and CRISPRTarget. Mojica et al. 2009. Microbiol. 155(Pt. 3):733-740; Atschul et al. 1990. J. Mol. Biol. 215:403-410; Biswass et al. 2013 RNA Biol. 10:817-827; and Grissa et al. 2007. Nucleic Acid Res. 35:W52-57. Experimental approaches to PAM identification can include, but are not limited to, plasmid depletion assays (Jiang et al. 2013. Nat. Biotechnol. 31:233-239; Esvelt et al. 2013. Nat. Methods. 10:1116-1121; Kleinstiver et al. 2015. Nature. 523:481-485), screened by a high-throughput in vivo model called PAM-SCNAR (Pattanayak et al. 2013. Nat. Biotechnol. 31:839-843 and Leenay et al. 2016.Mol. Cell. 16:253), and negative screening (Zetsche et al. 2015. Cell. 163:759-771).

As previously mentioned, CRISPR-Cas systems that target RNA do not typically rely on PAM sequences. Instead, such systems typically recognize protospacer flanking sites (PFSs) instead of PAMs Thus, Type VI CRISPR-Cas systems typically recognize protospacer flanking sites (PFSs) instead of PAMs. PFSs represents an analogue to PAMs for RNA targets. Type VI CRISPR-Cas systems employ a Cas13. Some Cas13 proteins analyzed to date, such as Cas13a (C2c2) identified from Leptotrichia shahii (LShCAs13a) have a specific discrimination against G at the 3′end of the target RNA. The presence of a C at the corresponding crRNA repeat site can indicate that nucleotide pairing at this position is rejected. However, some Cas13 proteins (e.g., LwaCAs13a and PspCas13b) do not seem to have a PFS preference. See e.g., Gleditzsch et al. 2019. RNA Biology. 16(4):504-517.

Some Type VI proteins, such as subtype B, have 5′-recognition of D (G, T, A) and a 3′-motif requirement of NAN or NNA. One example is the Cas13b protein identified in Bergeyella zoohelcum (BzCas13b). See e.g., Gleditzsch et al. 2019. RNA Biology. 16(4):504-517.

Overall Type VI CRISPR-Cas systems appear to have less restrictive rules for substrate (e.g., target sequence) recognition than those that target DNA (e.g., Type V and type II).

### Sequences Related to Nucleus Targeting and Transportation

In some embodiments, one or more components (e.g., the Cas protein) in the composition for engineering cells may comprise one or more sequences related to nucleus targeting and transportation. Such sequences may facilitate the one or more components in the composition for targeting a sequence within a cell. In order to improve targeting of the CRISPR-Cas protein used in the methods of the present disclosure to the nucleus, it may be advantageous to provide one or both of these components with one or more nuclear localization sequences (NLSs).

In one example embodiment, the NLSs used in the context of the present disclosure are heterologous to the proteins. Non-limiting examples of NLSs include an NLS sequence derived from: the NLS of the SV40 virus large T-antigen, having the amino acid sequence PKKKRKV (SEQ ID NO:1) or PKKKRKVEAS (SEQ ID NO:2); the NLS from nucleoplasmin (e.g., the nucleoplasmin bipartite NLS with the sequence KRPAATKKAGQAKKKK (SEQ ID NO:3)); the c-myc NLS having the amino acid sequence PAAKRVKLD (SEQ ID NO:4) or RQRRNELKRSP (SEQ ID NO:5); the hRNPAI M9 NLS having the sequence NQSSNFGPMKGGNFGGRSSGPYGGGGQYFAKPRNQGGY (SEQ ID NO:6); the sequence RMRIZFKNKGKDTAELRRRRVEVSVELRKAKKDEQILKRRNV (SEQ ID NO:7) of the IBB domain from importin-alpha; the sequences VSRKRPRP (SEQ ID NO:8) and PPKKARED (SEQ ID NO:9) of the myoma T protein; the sequence PQPKKKPL (SEQ ID NO:10) of human p53; the sequence SALIKKKKKMAP (SEQ ID NO:11) of mouse c-abl IV; the sequences DRLRR (SEQ ID NO:12) and PKQKKRK (SEQ ID NO:13) of the influenza virus NS1; the sequence RKLKKKIKKL (SEQ ID NO:14) of the Hepatitis virus delta antigen; the sequence REKKKFLKRR (SEQ ID NO:15) of the mouse Mx1 protein; the sequence KRKGDEVDGVDEVAKKKSKK (SEQ ID NO:16) of the human poly(ADP-ribose) polymerase; and the sequence RKCLQAGMNLEARKTKK (SEQ ID NO:17) of the steroid hormone receptors (human) glucocorticoid. In general, the one or more NLSs are of sufficient strength to drive accumulation of the DNA-targeting Cas protein in a detectable amount in the nucleus of a eukaryotic cell. In general, strength of nuclear localization activity may derive from the number of NLSs in the CRISPR-Cas protein, the particular NLS(s) used, or a combination of these factors. Detection of accumulation in the nucleus may be performed by any suitable technique. For example, a detectable marker may be fused to the nucleic acid-targeting protein, such that location within a cell may be visualized, such as in combination with a means for detecting the location of the nucleus (e.g., a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of nucleic acid-targeting complex formation (e.g., assay for deaminase activity) at the target sequence, or assay for altered gene expression activity affected by DNA-targeting complex formation and/or DNA-targeting), as compared to a control not exposed to the Cas protein, or exposed to a Cas protein lacking the one or more NLSs.

The Cas proteins may be provided with 1 or more, such as with, 2, 3, 4, 5, 6, 7, 8, 9, 10, or more heterologous NLSs. In some embodiments, the proteins comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. In preferred embodiments of the Cas proteins, an NLS attached to the C-terminal of the protein.

In certain embodiments, the CRISPR-Cas protein and a functional domain protein (described further herein) are delivered to the cell or expressed within the cell as separate proteins. In these embodiments, each of the CRISPR-Cas and functional domain protein can be provided with one or more NLSs as described herein. In certain embodiments, the CRISPR-Cas and functional domain protein are delivered to the cell or expressed with the cell as a fusion protein. In these embodiments one or both of the CRISPR-Cas and functional domain protein is provided with one or more NLSs. Where the functional domain protein is fused to an adaptor protein (such as MS2) as described above, the one or more NLS can be provided on the adaptor protein, provided that this does not interfere with aptamer binding. In particular embodiments, the one or more NLS sequences may also function as linker sequences between the functional domain protein and the CRISPR-Cas protein.

In certain embodiments, guides of the disclosure comprise specific binding sites (e.g. aptamers) for adapter proteins, which may be linked to or fused to a functional domain protein or catalytic domain thereof. When such a guide forms a CRISPR complex (e.g., CRISPR-Cas protein binding to guide and target), the adapter proteins bind and the functional domain protein or catalytic domain thereof associated with the adapter protein is positioned in a spatial orientation which is advantageous for the attributed function to be effective.

The skilled person will understand that modifications to the guide which allow for binding of the adapter+nucleotide deaminase, but not proper positioning of the adapter+nucleotide deaminase (e.g., due to steric hindrance within the three-dimensional structure of the CRISPR complex) are modifications which are not intended. The one or more modified guide may be modified at the tetra loop, the stem loop 1, stem loop 2, or stem loop 3, as described herein, preferably at either the tetra loop or stem loop 2, and in some cases at both the tetra loop and stem loop 2.

In some embodiments, a component (e.g., the dead Cas protein, the functional domain protein or catalytic domain thereof, or a combination thereof) in the systems may comprise one or more nuclear export signals (NES), one or more nuclear localization signals (NLS), or any combinations thereof. In some cases, the NES may be an HIV Rev NES. In certain cases, the NES may be MAPK NES. When the component is a protein, the NES or NLS may be at the C terminus of component. Alternatively, or additionally, the NES or NLS may be at the N terminus of component. In some examples, the Cas protein and optionally said functional domain protein or catalytic domain thereof comprise one or more heterologous nuclear export signal(s) (NES(s)) or nuclear localization signal(s) (NLS(s)), preferably an HIV Rev NES or MAPK NES, preferably C-terminal.

### CRISPR-Cas Cleavage

In one example embodiment, the CRISPR-Cas system may induce a double- or single-stranded break at a designated site in the target sequence. The CRISPR-Cas system may introduce an indel, which, as used herein, refers to insertions or deletions of the DNA at particular locations on the chromosome. The site of CRISPR-Cas cleavage, for most CRISPR-Cas systems, is dictated by distance from a protospacer-adjacent motif (PAM). Accordingly, a guide sequence may be selected to direct the CRISPR-Cas system to induce cleavage at a desired target site at or near the one or more variants.

### NHEJ-Based Editing

In one example embodiment, the CRISPR-Cas system is used to introduce one or more insertions or deletions to a target sequence on the gene or enhancer associated with the gene such that one or more indels or insertions reduce expression or activity of the one or more polypeptides. More than one guide sequence may be selected to insert multiple insertion, deletions, or combination thereof. Likewise, more than one Cas protein type may be used, for example, to maximize targets sites adjacent to different PAMs. In one example embodiment, a guide sequence is selected that directs the CRISPR-Cas system to make one or more insertions or deletions within the enhancer region. In one example embodiment, a guide is selected that directs the CRISPR-Cas system to make an insertion 1, 2, 3, 4, 5, 6, 7, 8, 9, 10, 11, 12, 13, 14, 15, 16, 17, 18, 19, 20, 21, 22, 23, 24, or 25 base pairs upstream of an enhancer controlling expression of a target gene. In one example embodiment, a guide sequence is selected to that directs the CRISPR-Cas system to make an insertion 1, 2, 3, 4, 5, 6, 7, 8, 9, 10, 11, 12, 13, 14, 15, 16, 17, 18, 19, 20, 21, 22, 23, 24, or 25 base pairs downstream of an enhancer controlling expression of a target gene. In one example embodiment, a guide sequence is selected that directs the CRISPR-Cas system to make a deletion 1, 2, 3, 4, 5, 6, 7, 8, 9, 10, 11, 12, 13, 14, 15, 16, 17, 18, 19, 20, 21, 22, 23, 24, or 25 base pairs downstream of an enhancer controlling expression of a target gene. In one example embodiment, a guide sequence is selected that directs the CRISPR-Cas system to make a deletion 1, 2, 3, 4, 5, 6, 7, 8, 9, 10, 11, 12, 13, 14, 15, 16, 17, 18, 19, 20, 21, 22, 23, 24, or 25 base pairs downstream of an enhancer controlling expression of a target gene.

### HDR Template Based Editing

In one example embodiment, a donor template is provided to replace a genomic sequence in a target gene or sequence controlling expression of the target gene. A donor template may comprise an insertion sequence flanked by two homology regions. The insertion sequence comprises an edited sequence to be inserted in place of the target sequence (e.g. a portion of genomic DNA to be edited). The homology regions comprise sequences that are homologous to the genomic DNA strands at the site of the CRISPR-Cas induced double-strand break. Cellular HDR mechanisms then facilitate insertion of the insertion sequence at the site of the DSB.

Accordingly, in certain example embodiments, a donor template and guide sequence are selected to direct excision and replacement of a section of genome DNA comprising an enhancer controlling expression of a target gene or a section of genome DNA within the gene that is required for activity of the target gene. In one example embodiment, the insertion sequence comprises a transcription factor binding site that recruits a repressor to the gene.

The donor template may include a sequence which results in a change in sequence of 1, 2, 3, 4, 5, 6, 7, 8, 9, 10, 11, 12 or more nucleotides of the target sequence.

A donor template may be of any suitable length, such as about or more than about 10, 15, 20, 25, 50, 75, 100, 150, 200, 500, 1000, or more nucleotides in length. In an embodiment, the template nucleic acid may be 20+/−10, 30+/−10, 40+/−10, 50+/−10, 60+/−10, 70+/−10, 80+/−10, 90+/−10, 100+/−10, 110+/−10, 120+/−10, 130+/−10, 140+/−10, 150+/−10, 160+/−10, 170+/−10, 180+/−10, 190+/−10, 200+/−10, 210+/−10, of 220+/−10 nucleotides in length. In an embodiment, the template nucleic acid may be 30+/−20, 40+/−20, 50+/−20, 60+/−20, 70+/−20, 80+/−20, 90+/−20, 100+/−20, 110+/−20, 120+/−20, 130+/−20, 140+/−20, I 50+/−20, 160+/−20, 170+/−20, 180+/−20, 190+/−20, 200+/−20, 210+/−20, of 220+/−20 nucleotides in length. In an embodiment, the template nucleic acid is 10 to 1,000, 20 to 900, 30 to 800, 40 to 700, 50 to 600, 50 to 500, 50 to 400, 50 to 300, 50 to 200, or 50 to 100 nucleotides in length.

The homology regions of the donor template may be complementary to a portion of a polynucleotide comprising the target sequence. When optimally aligned, a donor template might overlap with one or more nucleotides of a target sequences (e.g. about or more than about 1, 5, 10, 15, 20, 25, 30, 35, 40, 45, 50, 60, 70, 80, 90, 100 or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence.

The donor template comprises a sequence to be integrated (e.g., a mutated gene). The sequence for integration may be a sequence endogenous or exogenous to the cell. Examples of a sequence to be integrated include polynucleotides encoding a protein or a non-coding RNA (e.g., a microRNA). Thus, the sequence for integration may be operably linked to an appropriate control sequence or sequences. Alternatively, the sequence to be integrated may provide a regulatory function.

Homology arms of the donor template may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000.

In one example embodiment, one or both homology arms may be shortened to avoid including certain sequence repeat elements. For example, a 5′ homology arm may be shortened to avoid a sequence repeat element. In other embodiments, a 3′ homology arm may be shortened to avoid a sequence repeat element. In some embodiments, both the 5′ and the 3′ homology arms may be shortened to avoid including certain sequence repeat elements.

The donor template may further comprise a marker. Such a marker may make it easy to screen for targeted integrations. Examples of suitable markers include restriction sites, fluorescent proteins, or selectable markers. The donor template of the disclosure can be constructed using recombinant techniques (see, for example, Sambrook et al., 2001 and Ausubel et al., 1996).

In one example embodiment, a donor template is a single-stranded oligonucleotide. When using a single-stranded oligonucleotide, 5′ and 3′ homology arms may range up to about 200 base pairs (bp) in length, e.g., at least 25, 50, 75, 100, 125, 150, 175, or 200 bp in length.

Suzuki et al. describe in vivo genome editing via CRISPR/Cas9 mediated homology-independent targeted integration (2016, Nature 540:144-149).

### Templates

In some embodiments, a composition for engineering cells comprises a template, e.g., a recombination template. A template may be a component of another vector as described herein, contained in a separate vector, or provided as a separate polynucleotide. In some embodiments, a recombination template is designed to serve as a template in homologous recombination, such as within or near a target sequence nicked or cleaved by a nucleic acid-targeting effector protein as a part of a nucleic acid-targeting complex.

In an embodiment, the template nucleic acid alters the sequence of the target position. In an embodiment, the template nucleic acid results in the incorporation of a modified, or non-naturally occurring base into the target nucleic acid.

The template sequence may undergo a breakage mediated or catalyzed recombination with the target sequence. In an embodiment, the template nucleic acid may include sequence that corresponds to a site on the target sequence that is cleaved by a Cas protein mediated cleavage event. In an embodiment, the template nucleic acid may include a sequence that corresponds to both, a first site on the target sequence that is cleaved in a first Cas protein mediated event, and a second site on the target sequence that is cleaved in a second Cas protein mediated event.

In certain embodiments, the template nucleic acid can include a sequence which results in an alteration in the coding sequence of a translated sequence, e.g., one which results in the substitution of one amino acid for another in a protein product, e.g., transforming a mutant allele into a wild type allele, transforming a wild type allele into a mutant allele, and/or introducing a stop codon, insertion of an amino acid residue, deletion of an amino acid residue, or a nonsense mutation. In certain embodiments, the template nucleic acid can include a sequence which results in an alteration in a non-coding sequence, e.g., an alteration in an exon or in a 5′ or 3′ non-translated or non-transcribed region. Such alterations include an alteration in a control element, e.g., a promoter, enhancer, and an alteration in a cis-acting or trans-acting control element.

A template nucleic acid having homology with a target position in a target gene may be used to alter the structure of a target sequence. The template sequence may be used to alter an unwanted structure, e.g., an unwanted or mutant nucleotide. The template nucleic acid may include a sequence which, when integrated, results in decreasing the activity of a positive control element; increasing the activity of a positive control element; decreasing the activity of a negative control element; increasing the activity of a negative control element; decreasing the expression of a gene; increasing the expression of a gene; increasing resistance to a disorder or disease; increasing resistance to viral entry; correcting a mutation or altering an unwanted amino acid residue conferring, increasing, abolishing or decreasing a biological property of a gene product, e.g., increasing the enzymatic activity of an enzyme, or increasing the ability of a gene product to interact with another molecule.

The template nucleic acid may include a sequence which results in a change in sequence of 1, 2, 3, 4, 5, 6, 7, 8, 9, 10, 11, 12 or more nucleotides of the target sequence.

A template polynucleotide may be of any suitable length, such as about or more than about 10, 15, 20, 25, 50, 75, 100, 150, 200, 500, 1000, or more nucleotides in length. In an embodiment, the template nucleic acid may be 20+/−10, 30+/−10, 40+/−10, 50+/−10, 60+/−10, 70+/−10, 80+/−10, 90+/−10, 100+/−10, 110+/−10, 120+/−10, 130+/−10, 140+/−10, 150+/−10, 160+/−10, 170+/−10, 180+/−10, 190+/−10, 200+/−10, 210+/−10, of 220+/−10 nucleotides in length. In an embodiment, the template nucleic acid may be 30+/−20, 40+/−20, 50+/−20, 60+/−20, 70+/−20, 80+/−20, 90+/−20, 100+/−20, 110+/−20, 120+/−20, 130+/−20, 140+/−20, 150+/−20, 160+/−20, 170+/−20, 180+/−20, 190+/−20, 200+/−20, 210+/−20, of 220+/−20 nucleotides in length. In an embodiment, the template nucleic acid is 10 to 1,000, 20 to 900, 30 to 800, 40 to 700, 50 to 600, 50 to 500, 50 to 400, 50 to 300, 50 to 200, or 50 to 100 nucleotides in length.

In some embodiments, the template polynucleotide is complementary to a portion of a polynucleotide comprising the target sequence. When optimally aligned, a template polynucleotide might overlap with one or more nucleotides of a target sequences (e.g. about or more than about 1, 5, 10, 15, 20, 25, 30, 35, 40, 45, 50, 60, 70, 80, 90, 100 or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence.

The exogenous polynucleotide template comprises a sequence to be integrated (e.g., a mutated gene). The sequence for integration may be a sequence endogenous or exogenous to the cell. Examples of a sequence to be integrated include polynucleotides encoding a protein or a non-coding RNA (e.g., a microRNA). Thus, the sequence for integration may be operably linked to an appropriate control sequence or sequences. Alternatively, the sequence to be integrated may provide a regulatory function.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000

In certain embodiments, one or both homology arms may be shortened to avoid including certain sequence repeat elements. For example, a 5′ homology arm may be shortened to avoid a sequence repeat element. In other embodiments, a 3′ homology arm may be shortened to avoid a sequence repeat element. In some embodiments, both the 5′ and the 3′ homology arms may be shortened to avoid including certain sequence repeat elements.

In some methods, the exogenous polynucleotide template may further comprise a marker. Such a marker may make it easy to screen for targeted integrations. Examples of suitable markers include restriction sites, fluorescent proteins, or selectable markers. The exogenous polynucleotide template of the disclosure can be constructed using recombinant techniques (see, for example, Sambrook et al., 2001 and Ausubel et al., 1996).

In certain embodiments, a template nucleic acid for correcting a mutation may designed for use as a single-stranded oligonucleotide. When using a single-stranded oligonucleotide, 5′ and 3′ homology arms may range up to about 200 base pairs (bp) in length, e.g., at least 25, 50, 75, 100, 125, 150, 175, or 200 bp in length.

Suzuki et al. describe in vivo genome editing via CRISPR/Cas9 mediated homology-independent targeted integration (2016, Nature 540:144-149).

### Specialized Cas-Based Systems

In some embodiments, the system is a Cas-based system that is capable of performing a specialized function or activity. For example, the Cas protein may be fused, operably coupled to, or otherwise associated with one or more functionals domains. In certain example embodiments, the Cas protein may be a catalytically dead Cas protein (“dCas”) and/or have nickase activity. A nickase is a Cas protein that cuts only one strand of a double stranded target. In such embodiments, the dCas or nickase provide a sequence specific targeting functionality that delivers the functional domain to or proximate a target sequence. Example functional domains that may be fused to, operably coupled to, or otherwise associated with a Cas protein can be or include, but are not limited to a nuclear localization signal (NLS) domain, a nuclear export signal (NES) domain, a translational activation domain, a transcriptional activation domain (e.g. VP64, p65, MyoD1, HSF1, RTA, and SET7/9), a translation initiation domain, a transcriptional repression domain (e.g., a KRAB domain, NuE domain, NcoR domain, and a SID domain such as a SID4X domain), a nuclease domain (e.g., FokI), a histone modification domain (e.g., a histone acetyltransferase), a light inducible/controllable domain, a chemically inducible/controllable domain, a transposase domain, a homologous recombination machinery domain, a recombinase domain, an integrase domain, and combinations thereof. Methods for generating catalytically dead Cas9 or a nickase Cas9 (WO 2014/204725, Ran et al. Cell. 2013 Sep. 12; 154(6):1380-1389), Cas12 (Liu et al. Nature Communications, 8, 2095 (2017), and Cas13 (International Patent Publication Nos. WO 2019/005884 and WO2019/060746) are known in the art and incorporated herein by reference.

In some embodiments, the functional domains can have one or more of the following activities: methylase activity, demethylase activity, translation activation activity, translation initiation activity, translation repression activity, transcription activation activity, transcription repression activity, transcription release factor activity, histone modification activity, nuclease activity, single-strand RNA cleavage activity, double-strand RNA cleavage activity, single-strand DNA cleavage activity, double-strand DNA cleavage activity, molecular switch activity, chemical inducibility, light inducibility, and nucleic acid binding activity. In some embodiments, the one or more functional domains may comprise epitope tags or reporters. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporters include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and auto-fluorescent proteins including blue fluorescent protein (BFP).

The one or more functional domain(s) may be positioned at, near, and/or in proximity to a terminus of the effector protein (e.g., a Cas protein). In embodiments having two or more functional domains, each of the two can be positioned at or near or in proximity to a terminus of the effector protein (e.g., a Cas protein). In some embodiments, such as those where the functional domain is operably coupled to the effector protein, the one or more functional domains can be tethered or linked via a suitable linker (including, but not limited to, GlySer linkers) to the effector protein (e.g., a Cas protein). When there is more than one functional domain, the functional domains can be same or different. In some embodiments, all the functional domains are the same. In some embodiments, all of the functional domains are different from each other. In some embodiments, at least two of the functional domains are different from each other. In some embodiments, at least two of the functional domains are the same as each other.

Other suitable functional domains can be found, for example, in International Patent Publication No. WO 2019/018423.

### Slit CRISPR-Cas Systems

In one example embodiment, the CRISPR-Cas system is a split CRISPR-Cas system. See e.g., Zetche et al., 2015. Nat. Biotechnol. 33(2): 139-142 and International Patent Publication WO 2019/018423, the compositions and techniques of which can be used in and/or adapted for use with the present disclosure. Split CRISPR-Cas proteins are set forth herein and in documents incorporated herein by reference in further detail herein. In certain embodiments, each part of a split CRISPR protein is attached to a member of a specific binding pair, and when bound with each other, the members of the specific binding pair maintain the parts of the CRISPR protein in proximity. In certain embodiments, each part of a split CRISPR protein is associated with an inducible binding pair. An inducible binding pair is one which is capable of being switched “on” or “off” by a protein or small molecule that binds to both members of the inducible binding pair. In some embodiments, CRISPR proteins may preferably split between domains, leaving domains intact. In particular embodiments, said Cas split domains (e.g., RuvC and HNH domains in the case of Cas9) can be simultaneously or sequentially introduced into the cell such that said split Cas domain(s) process the target nucleic acid sequence in the algae cell. The reduced size of the split Cas compared to the wild type Cas allows other methods of delivery of the systems to the cells, such as the use of cell penetrating peptides as described herein.

### DNA and RNA Base Editing

In one example embodiment, the gene editing system configured to modify the one or more target genes disclosed herein is a base editing system. In one example embodiment, a Cas protein is connected or fused to a nucleotide deaminase. As used herein, “base editing” refers generally to the process of polynucleotide modification via a CRISPR-Cas-based or Cas-based system that does not include excising nucleotides to make the modification. Base editing can convert base pairs at precise locations without generating excess undesired editing byproducts that can be made using traditional CRISPR-Cas systems. Accordingly, in one example embodiment, the base editing system edits the target gene to reduce or eliminate its expression.

In one example embodiment, the nucleotide deaminase may be a DNA base editor used in combination with a DNA binding Cas protein such as, but not limited to, Class 2 Type II and Type V systems. Two classes of DNA base editors are generally known: cytosine base editors (CBEs) and adenine base editors (ABEs). CBEs convert a C⋅G base pair into a T⋅A base pair (Komor et al. 2016. Nature. 533:420-424; Nishida et al. 2016. Science. 353; and Li et al. Nat. Biotech. 36:324-327) and ABEs convert an A⋅T base pair to a G⋅C base pair. Collectively, CBEs and ABEs can mediate all four possible transition mutations (C to T, A to G, T to C, and G to A). Rees and Liu. 2018. Nat. Rev. Genet. 19(12): 770-788, particularly at FIGS. 1b, 2a-2c, 3a-3f, and Table 1. In some embodiments, the base editing system includes a CBE and/or an ABE. In some embodiments, a polynucleotide of the present disclosure described elsewhere herein can be modified using a base editing system. Rees and Liu. 2018. Nat. Rev. Gent. 19(12):770-788. Base editors also generally do not need a DNA donor template and/or rely on homology-directed repair. Komor et al. 2016. Nature. 533:420-424; Nishida et al. 2016. Science. 353; and Gaudeli et al. 2017. Nature. 551:464-471. Upon binding to a target locus in the DNA, base pairing between the guide RNA of the system and the target DNA strand leads to displacement of a small segment of ssDNA in an “R-loop”. Nishimasu et al. Cell. 156:935-949. DNA bases within the ssDNA bubble are modified by the enzyme component, such as a deaminase. In some systems, the catalytically disabled Cas protein can be a variant or modified Cas can have nickase functionality and can generate a nick in the non-edited DNA strand to induce cells to repair the non-edited strand using the edited strand as a template. Komor et al. 2016. Nature. 533:420-424; Nishida et al. 2016. Science. 353; and Gaudeli et al. 2017. Nature. 551:464-471.

Other Example Type V base editing systems are described in International Patent Publication Nos. WO 2018/213708, WO 2018/213726, and International Patent Applications No. PCT/US2018/067207, PCT/US2018/067225, and PCT/US2018/067307, each of which is incorporated herein by reference.

In one example embodiment, the base editing system may be an RNA base editing system. As with DNA base editors, a nucleotide deaminase capable of converting nucleotide bases may be fused to a Cas protein. However, in these embodiments, the Cas protein will need to be capable of binding RNA. Example RNA binding Cas proteins include, but are not limited to, RNA-binding Cas9s such as Francisella novicida Cas9 (“FnCas9”), and Class 2 Type VI Cas systems. The nucleotide deaminase may be a cytidine deaminase or an adenosine deaminase, or an adenosine deaminase engineered to have cytidine deaminase activity. In certain example embodiments, the RNA base editor may be used to delete or introduce a post-translation modification site in the expressed mRNA. In contrast to DNA base editors, whose edits are permanent in the modified cell, RNA base editors can provide edits where finer, temporal control may be needed, for example in modulating a particular immune response. Example Type VI RNA-base editing systems are described in Cox et al. 2017. Science 358: 1019-1027, International Patent Publication Nos. WO 2019/005884, WO 2019/005886, and WO 2019/071048, and International Patent Application Nos. PCT/US20018/05179 and PCT/US2018/067207, which are incorporated herein by reference. An example FnCas9 system that may be adapted for RNA base editing purposes is described in International Patent Publication No. WO 2016/106236, which is incorporated herein by reference.

An example method for delivery of base-editing systems, including use of a split-intein approach to divide CBE and ABE into reconstitutable halves, is described in Levy et al. Nature Biomedical Engineering doi.org/10.1038/s41441-019-0505-5 (2019), which is incorporated herein by reference.

### Prime Editors

In one example embodiment, the gene editing system configured to modify the target genes is a prime editing system. See e.g. Anzalone et al. 2019. Nature. 576: 149-157; and International patent application publication No. WO2022150790A2. Prime editing advantageously provides lower off-target editing than a Cas9 nuclease system. In example embodiments, the target gene is edited to introduce a stop codon, mutate an essential residue (e.g., an active site residue in a target enzyme, a residue essential for protein-protein binding, or a residue required for modification), or introduce a frameshift that inactivates the gene. In example a regulatory sequence, such as an enhancer sequence is edited to reduce or eliminate binding of a transcription factor.

In one example embodiment, a genomic sequence in a target gene or sequence controlling expression of the target gene is replaced or deleted using a prime editing system. Like base editing systems, prime editing systems can be capable of targeted modification of a polynucleotide without generating double stranded breaks. Further prime editing systems are capable of all 12 possible combination swaps. Prime editing may operate via a “search-and-replace” methodology and can mediate targeted insertions, deletions, of all 12 possible base-to-base conversion and combinations thereof. Generally, a prime editing system, as exemplified by PE1, PE2, and PE3 (Id), can include a reverse transcriptase fused or otherwise coupled or associated with an RNA-programmable nickase and a prime-editing extended guide RNA (pegRNA) to facility direct copying of genetic information from the extension on the pegRNA into the target polynucleotide. Embodiments that can be used with the present disclosure include these and variants thereof. Prime editing can have the advantage of lower off-target activity.

In some embodiments, the prime editing guide molecule can specify both the target polynucleotide information (e.g., sequence) and contain a new polynucleotide cargo that replaces target polynucleotides. To initiate transfer from the guide molecule to the target polynucleotide, the PE system can nick the target polynucleotide at a target side to expose a 3′hydroxyl group, which can prime reverse transcription of an edit-encoding extension region of the guide molecule (e.g., a prime editing guide molecule or peg guide molecule) directly into the target site in the target polynucleotide. See e.g., Anzalone et al. 2019. Nature. 576: 149-157, particularly at FIGS. 1b, 1c, related discussion, and Supplementary discussion.

In some embodiments, a prime editing system can be composed of a Cas polypeptide having nickase activity, a reverse transcriptase, and a guide molecule. The Cas polypeptide can lack nuclease activity. The guide molecule can include a target binding sequence as well as a primer binding sequence and a template containing the edited polynucleotide sequence. The guide molecule, Cas polypeptide, and/or reverse transcriptase can be coupled together or otherwise associate with each other to form an effector complex and edit a target sequence. In some embodiments, the Cas polypeptide is a Class 2, Type V Cas polypeptide. In some embodiments, the Cas polypeptide is a Cas9 polypeptide (e.g., is a Cas9 nickase). In some embodiments, the Cas polypeptide is fused to the reverse transcriptase. In some embodiments, the Cas polypeptide is linked to the reverse transcriptase.

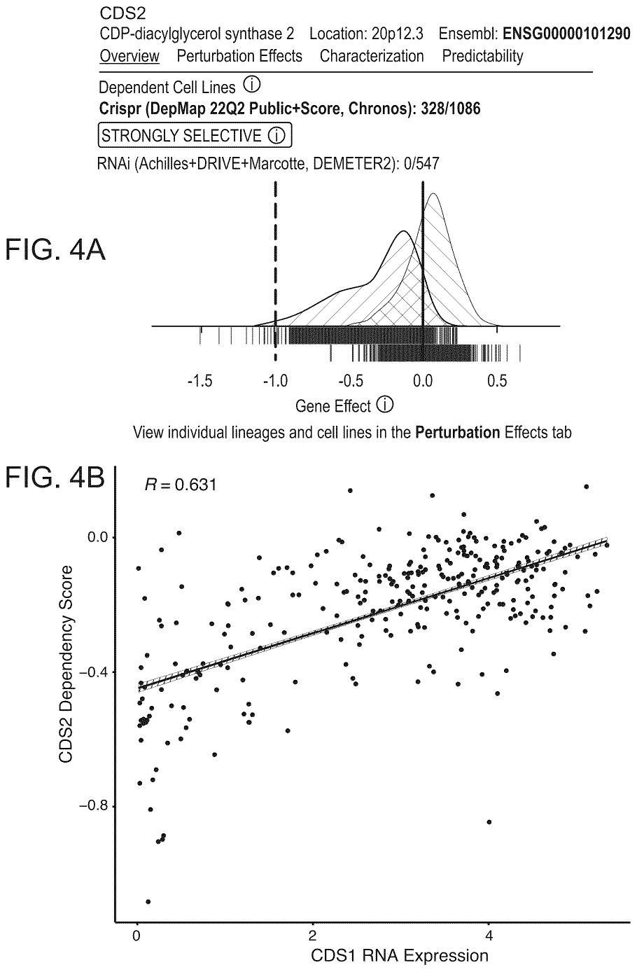

In some embodiments, the prime editing system can be a PE1 system or variant thereof, a PE2 system or variant thereof, or a PE3 (e.g., PE3, PE3b) system. See e.g., Anzalone et al. 2019. Nature. 576: 149-157, particularly at pgs. 2-3, FIGS. 2a, 3a-3f, 4a-4b, Extended data FIGS. 3a-3b, 4,

The peg guide molecule can be about 10 to about 200 or more nucleotides in length, such as 10 to/or 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 or more nucleotides in length. Optimization of the peg guide molecule can be accomplished as described in Anzalone et al. 2019. Nature. 576: 149-157, particularly at pg. 3, FIG. 2a-2b, and Extended Data FIGS. 5a-c.

Prime editing can also include a system that uses a prime editor (PE) protein and two prime editing guide RNAs (pegRNAs), such that, the two pegRNAs template the synthesis of complementary DNA flaps on opposing strands of genomic DNA, which replace the endogenous DNA sequence between the PE-induced nick sites. See, e.g., Anzalone A V, Gao X D, Podracky C J, et al. Programmable deletion, replacement, integration and inversion of large DNA sequences with twin prime editing. Nat Biotechnol. 2022; 40(5):731-740. Thus, use of two pegRNAs allows for larger insertions or deletions because of the two overlapping 3′ flaps created by the two nicked sites. The system can be combined with a site-specific serine recombinase to allow targeted integration of gene-sized DNA plasmids (>5,000 bp) and targeted sequence inversions of 40 kb in human cells. Id. In one example embodiment, the system can be used to insert or replace a sequence into one or more target genes. In example embodiments, the insertion or replacement results in an inactive target gene or less active form of the target gene. In one example embodiment, the system is used to replace all or a portion of the entire target gene. In one example embodiment, the system is used to replace all or a portion of an enhancer controlling the target gene expression.

### CRISPR-Directed Integrase

In example embodiments, the prime editing system inserts a serine integrase attachment site for large, multiplexed gene insertion without reliance on DNA repair pathways. See, e.g., Yarnall M T N, Ioannidi E I, Schmitt-Ulms C, et al. Drag-and-drop genome insertion of large sequences without double-strand DNA cleavage using CRISPR-directed integrases [published online ahead of print, 2022 Nov. 24] Nat Biotechnol. 2022. This system is a variation of prime editing that includes all of the components of prime editing, but with an integrase. Serine integrases typically insert sequences containing an attP attachment site into a target containing the related attB attachment site. By using programmable genome editing to place integrase landing sites at desired locations in the genome, this system directly guides the activity of the associated integrase to the specific genomic site. In one embodiment, pegRNAs including attB sequences are used to insert the sites at desired locations in the genome. In one embodiment, the system uses a Cas enzyme-reverse transcriptase-integrase fusion protein to directly recruit the integrase to the target site.

“Uni-directional recombinases” or “integrases” refer to recombinase enzymes whose recognition sites are destroyed after the recombination has taken place. The term “integrase” refers to a type of recombinase. In other words, the sequence recognized by the recombinase is changed into one that is not recognized by the recombinase upon recombination. As a result, once a sequence is subjected to recombination by the uni-directional recombinase, the continued presence of the recombinase cannot reverse the previous recombination event.

Typically, two different sites are involved (in regards to recombination termed “complementary sites”), one present in the target nucleic acid (e.g., a chromosome or episome of a eukaryote) and another on the nucleic acid that is to be integrated at the target recombination site. The terms “attB” and “attP,” which refer to attachment (or recombination) sites originally from a bacterial target (attachment site of bacteria) and a phage donor (attachment site of phage), respectively, are used herein although recombination sites for particular enzymes may have different names. The two attachment sites can share as little sequence identity as a few base pairs. The recombination sites typically include left and right arms separated by a core or spacer region. Thus, an attB recombination site consists of BOB′, where B and B′ are the left and right arms, respectively, and O is the core region. Similarly, attP is POP′, where P and P′ are the arms and O is again the core region. Upon recombination between the attB and attP sites, and concomitant integration of a nucleic acid at the target, the recombination sites that flank the integrated DNA are referred to as “attL” and “attR.” The attL and attR sites, using the terminology above, thus consist of BOP′ and POB′, respectively. In some representations herein, the “O” is omitted and attB and attP, for example, are designated as BB′ and PP′, respectively.

In example embodiments, the recombinase of the present disclosure is a serine integrase. In example embodiments, serine integrases specifically recombine when recognizing the two attachment sites specific for the integrase. In example embodiments, the heterologous sites are referred to as attP and attB, however, these terms refer to the specific sequences recognized by the specific integrase and do not refer to a single consensus sequence. Serine integrases mediate site-specific recombination between short recognition sites located in phage genomes and bacterial chromosomes, respectively, the attachment site of phage (attP) and attachment site of bacteria (attB) (i.e., the target sites of the integrase), to form the hybrid attachment sites attL and attR. Unlike Cre and Flp recombinases that catalyze reversible site-specific recombination reactions, serine integrases are unidirectional and catalyze only attP and attB recombination without RDF or Xis accessory proteins. Thus, in the absence of any accessory factors, integrase is unidirectional. In addition, DNA substrates identified by serine integrases (attP and attB) are relatively short (30-50 bp) and have a minimal length of approximately 34-40 base pairs (bp) (Groth A C et al., Proc. Natl. Acad. Sci. USA 97, 5995-6000 (2000)). The compatibility of distinct DNA topological structures is also quite different from recognition of DNA by Hin recombinase or Tn3 resolvase. Serine integrases recognize DNA substrates specifically, not at random, but can facilitate recombination at sequences with partial identity with wild-type recombination sites, termed pseudo attachment sites (either pseudo attP or pseudo attB). A “pseudo-recombination site” is a DNA sequence recognized by a recombinase enzyme such that the recognition site differs in one or more base pairs from the wild-type recombinase recognition sequence and/or is present as an endogenous sequence in a genome that differs from the genome where the wild-type recognition sequence for the recombinase resides. “Pseudo attP site” or “pseudo attB site” refer to pseudo sites that are similar to wild-type phage or bacterial attachment site sequences, respectively, for phage integrase enzymes. “Pseudo att site” is a more general term that can refer to either a pseudo attP site or a pseudo attB site. Specific attB and attP sequences for use in the present disclosure include all wildtype sequences as well as pseudo attB and attP sequences.

Recombination sites used in the present methods include those recognized by unidirectional, site-directed recombinases (e.g., integrases). Non-limiting examples of serine integrases and recombination sites applicable to the present disclosure include φC31 integrase, Bxb1, φBT1 integrase, A118, TP901-1, and R4 and the corresponding recombination sites for each (see, e.g., Groth, A. C. and Calos, M. P. (2004) J. Mol. Biol. 335, 667-678; Lei, et al., FEBS Lett. 2018 April; 592(8):1389-1399; Singh, et al., Attachment Site Selection and Identity in Bxb1 Serine Integrase-Mediated Site-Specific Recombination, PLoS Genet. 2013 May; 9(5):e1003490; and Gupta, et al., Nucleic Acids Res. 2007 May; 35(10): 3407-3419). Additional serine recombinases and recombination sites may be any of those disclosed in US 20180346934A1 and US 2010/0190178. In certain embodiments, a functional domain of the serine integrase is used.

In one example embodiment, the system can be used to insert or replace a sequence into one or more target genes. In example embodiments, the insertion or replacement results in an inactive target gene or less active form of the target gene. In one example embodiment, the system is used to replace all or a portion of the entire target gene. In one example embodiment, the system is used to replace all or a portion of an enhancer controlling the target gene expression.

### CRISPR Associated Transposase (CAST) Systems

In one example embodiment, the gene editing system configured to modify the one or more target genes is a CRISPR associated transposase system (CAST). In one example embodiment, the CAST system can be used to insert or replace a sequence into one or more target genes. In example embodiments, the insertion or replacement results in an inactive target gene or less active form of the target gene. In one example embodiment, a CAST system is used to replace all or a portion of an enhancer controlling the target gene expression. CAST system can include a Cas protein that is catalytically inactive, or engineered to be catalytically active, and further comprises a transposase (or subunits thereof) that catalyze RNA-guided DNA transposition. Such systems are able to insert DNA sequences at a target site in a DNA molecule without relying on host cell repair machinery. CAST systems can be Class 1 or Class 2 CAST systems. An example Class 1 system is described in Klompe et al. Nature, doi:10.1038/s41586-019-1323, which is in incorporated herein by reference. An example Class 2 system is described in Strecker et al. Science. 10/1126/science. aax9181 (2019), and PCT/US2019/066835 which are incorporated herein by reference.

### OMEGA Systems

In one example embodiment, the gene editing system configured to modify the one or more target genes is a transposon-encoded RNA-guided nuclease system, referred to herein as OMEGA (obligate mobile element-guided activity). See, e.g., Altae-Tran H, Kannan S, Demircioglu F E, et al. The widespread IS200/IS605 transposon family encodes diverse programmable RNA-guided endonucleases. Science. 2021; 374(6563):57-65. OMEGA systems include, but are not limited to IscB, IsrB, TnpB systems.

In some embodiments, the nucleic acid-guided nucleases herein may be an IscB protein (see, e.g., International patent application publication No. WO 2022/087494A1; and Altae-Tran H, et al. 2021). An IscB protein may comprise an X domain and a Y domain as described herein. In some examples, the IscB proteins may form a complex with one or more guide molecules. In some cases, the IscB proteins may form a complex with one or more hRNA molecules which serve as a scaffold molecule and comprise guide sequences. In some examples, the IscB proteins are CRISPR-associated proteins, e.g., the loci of the nucleases are associated with an CRISPR array. In some examples, the IscB proteins are not CRISPR-associated. In some examples, the IscB protein may be homolog or ortholog of IscB proteins described in Kapitonov V V et al., ISC, a Novel Group of Bacterial and Archaeal DNA Transposons That Encode Cas9 Homologs, J Bacteriol. 2015 Dec. 28; 198(5):797-807. doi: 10.1128/JB.00783-15, which is incorporated by reference herein in its entirety.

In some embodiments, the nucleic acid-guided nucleases herein may be an IsrB (Insertion sequence RuvC-like OrfB) protein (see, e.g., International patent application publication No. WO 2022/087494A1; and Altae-Tran H, et al. 2021). IsrB refers to a group of shorter, ˜350 aa IscB homologs that are also encoded in IS200/605 superfamily transposons. These proteins contain a PLMP domain and split RuvC but lack the HNH domain.

In some embodiments, the nucleic acid-guided nucleases herein may be a TnpB protein (see, e.g., International patent application publication No. WO 2022/159892A1; and Altae-Tran H, et al. 2021). TnpB is a putative endonuclease distantly related to IscB and thought to be the ancestor of Cas12, the type V CRISPR effector. The TnpB system comprises a TnpB polypeptide and a nucleic acid component capable of forming a complex with the TnpB polypeptide and directing the complex to a target polynucleotide. The TnpB systems and TnpB/nucleic acid component complexes may also be referred to herein as OMEGA (Obligate Mobile Element Guided Activity) systems or complexes, or W systems or complexes for short. TnpB systems are a distinct type of W system, which further include IscB, IsrB, and IshB systems. The nucleic acid component of W systems is structurally distinct from other RNA-guided nucleases, such as CRISPR-Cas systems, and may also be referred to as a wRNA. In certain example embodiments, the TnpB systems are RNA-predominate, that is the nucleic acid component makes a larger contribution to the overall size of the TnpB complex relative to other RNA-guided nuclease systems such as CRISPR-Cas. Also, given the more minimal structural features of TnpB relative other known programmable nucleases such as CRISPR-Cas, the polynucleotide binding pocket is open and more accessible, which can facilitate greater access to and ability to manipulate, modify, edit, remove, or delete nucleotides at a target region on the bound polynucleotide.

### Epigenetic Editing

In one example embodiment, the one or more agents is an epigenetic modification polypeptide comprising a DNA binding domain linked to or otherwise capable of associating with an epigenetic modification domain such that binding of the DNA binding domain at target sequence on genomic DNA (e.g., chromatin) results in one or more epigenetic modifications by the epigenetic modification domain that increases or decreases expression of the one or more polypeptides disclosed herein. As used herein, “linked to or otherwise capable of associating with” refers to a fusion protein or a recruitment domain or an adaptor protein, such as an aptamer (e.g., MS2) or an epitope tag. The recruitment domain or an adaptor protein can be linked to an epigenetic modification domain or the DNA binding domain (e.g., an adaptor for an aptamer). The epigenetic modification domain can be linked to an antibody specific for an epitope tag fused to the DNA binding domain. An aptamer can be linked to a guide sequence.

In example embodiments, the DNA binding domain is a programmable DNA binding protein linked to or otherwise capable of associating with an epigenetic modification domain. Programmable DNA binding proteins for modifying the epigenome include, but are not limited to CRISPR systems, transcription activator-like effectors (TALEs), Zn finger proteins and meganucleases (see, e.g., Thakore P I, Black J B, Hilton I B, Gersbach C A. Editing the epigenome: technologies for programmable transcription and epigenetic modulation. Nat Methods. 2016; 13(2):127-137; and described further herein). In example embodiments, the DNA binding domain is a nuclease-deficient RNA-guided DNA endonuclease enzyme or a nuclease-deficient endonuclease enzyme. In example embodiments, a CRISPR system having an inactivated nuclease activity (e.g., dCas) is used as the DNA binding domain.

In example embodiments, the epigenetic modification domain is a functional domain and includes, but is not limited to a histone methyltransferase (HMT) domain, histone demethylase domain, histone acetyltransferase (HAT) domain, histone deacetylation (HDAC) domain, DNA methyltransferase domain, DNA demethylation domain, histone phosphorylation domain (e.g., serine and threonine, or tyrosine), histone ubiquitylation domain, histone sumoylation domain, histone ADP ribosylation domain, histone proline isomerization domain, histone biotinylation domain, histone citrullination domain (see, e.g., Epigenetics, Second Edition, 2015, Edited by C. David Allis; Marie-Laure Caparros; Thomas Jenuwein; Danny Reinberg; Associate Editor Monika Lachlan; Dawson M A, Kouzarides T. Cancer epigenetics: from mechanism to therapy. Cell. 2012; 150(1):12-27; Syding L A, Nickl P, Kasparek P, Sedlacek R. CRISPR/Cas9 Epigenome Editing Potential for Rare Imprinting Diseases: A Review. Cells. 2020; 9(4):993; and Zhang Y. Transcriptional regulation by histone ubiquitination and deubiquitination. Genes Dev. 2003; 17(22):2733-2740). Example epigenetic modification domains can be obtained from, but are not limited to chromatin modifying enzymes, such as, DNA methyltransferases (e.g., DNMT1, DNMT3a and DNMT3b), TET1, TET2, thymine-DNA glycosylase (TDG), GCN5-related N-acetyltransferases family (GNAT), MYST family proteins (e.g., MOZ and MORF), and CBP/p300 family proteins (e.g., CBP, p300), Class I HDACs (e.g., HDAC 1-3 and HDAC8), Class II HDACs (e.g., HDAC 4-7 and HDAC 9-10), Class III HDACs (e.g., sirtuins), HDAC11, SET domain containing methyltransferases (e.g., SET7/9 (KMT7, NCBI Entrez Gene: 80854), KMT5A (SET8), MMSET, EZH2, and MLL family members), DOT1L, LSD1, Jumonji demethylases (e.g., KDM5A (JARID1A), KDM5C (JARIDIC), and KDM6A (UTX)), kinases (e.g., Haspin, VRK1, PKCα, PKCβ, PIM1, IKKα, Rsk2, PKB/Akt, Aurora B, MSK1/2, JNK1, MLTKα, PRK1, Chk1, Dlk/ZIP, PKCδ, MST1, AMPK, JAK2, Abl, BMK1, CaMK, S6K1, SIK1), Ubp8, ubiquitin C-terminal hydrolases (UCH), the ubiquitin-specific processing proteases (UBP), and poly(ADP-ribose) polymerase 1 (PARP-1). See, also, U.S. Pat. No. 11,001,829B2 for additional domains.

In example embodiments, histone acetylation is targeted to a target sequence using a CRISPR system (see, e.g., Hilton I B, et al. Epigenome editing by a CRISPR-Cas9-based acetyltransferase activates genes from promoters and enhancers. Nat Biotechnol. 2015). In example embodiments, histone deacetylation is targeted to a target sequence (see, e.g., Cong et al., 2012; and Konermann S, et al. Optical control of mammalian endogenous transcription and epigenetic states. Nature. 2013; 500:472-476). In example embodiments, histone methylation is targeted to a target sequence (see, e.g., Snowden A W, Gregory P D, Case C C, Pabo C O. Gene-specific targeting of H3K9 methylation is sufficient for initiating repression in vivo. Curr Biol. 2002; 12:2159-2166; and Cano-Rodriguez D, Gjaltema R A, Jilderda L J, et al. Writing of H3K4Me3 overcomes epigenetic silencing in a sustained but context-dependent manner. Nat Commun. 2016; 7:12284). In example embodiments, histone demethylation is targeted to a target sequence (see, e.g., Kearns N A, Pham H, Tabak B, et al. Functional annotation of native enhancers with a Cas9-histone demethylase fusion. Nat Methods. 2015; 12(5):401-403). In example embodiments, histone phosphorylation is targeted to a target sequence (see, e.g., Li J, Mahata B, Escobar M, et al. Programmable human histone phosphorylation and gene activation using a CRISPR/Cas9-based chromatin kinase. Nat Commun. 2021; 12(1):896). In example embodiments, DNA methylation is targeted to a target sequence (see, e.g., Rivenbark A G, et al. Epigenetic reprogramming of cancer cells via targeted DNA methylation. Epigenetics. 2012; 7:350-360; Siddique A N, et al. Targeted methylation and gene silencing of VEGF-A in human cells by using a designed Dnmt3a-Dnmt3L single-chain fusion protein with increased DNA methylation activity. J Mol Biol. 2013; 425:479-491; Bernstein D L, Le Lay J E, Ruano E G, Kaestner K H. TALE-mediated epigenetic suppression of CDKN2A increases replication in human fibroblasts. J Clin Invest. 2015; 125:1998-2006; Liu X S, Wu H, Ji X, et al. Editing DNA Methylation in the Mammalian Genome. Cell. 2016; 167(1):233-247.e17; Stepper P, Kungulovski G, Jurkowska R Z, et al. Efficient targeted DNA methylation with chimeric dCas9-Dnmt3a-Dnmt3L methyltransferase. Nucleic Acids Res. 2017; 45(4):1703-1713; and Pflueger C., Tan D., Swain T., Nguyen T., Pflueger J., Nefzger C., Polo J. M., Ford E., Lister R. A modular dCas9-SunTag DNMT3A epigenome editing system overcomes pervasive off-target activity of direct fusion dCas9-DNMT3A constructs. Genome Res. 2018; 28:1193-1206). In example embodiments, DNA demethylation is targeted to a target sequence using a CRISPR system (see, e.g., TET1, see Xu et al, Cell Discov. 2016 May 3; 2: 16009; Choudhury et al, Oncotarget. 2016 Jul. 19; 7(29):46545-46556; and Kang J G, Park J S, Ko J H, Kim Y S. Regulation of gene expression by altered promoter methylation using a CRISPR/Cas9-mediated epigenetic editing system. Sci Rep. 2019; 9(1):11960). In example embodiments, DNA demethylation is targeted to a target sequence (see, e.g., TDG, see, Gregory D J, Zhang Y, Kobzik L, Fedulov A V. Specific transcriptional enhancement of inducible nitric oxide synthase by targeted promoter demethylation. Epigenetics. 2013; 8:1205-1212).

Example epigenetic modification domains can be obtained from, but are not limited to transcription activators, such as, VP64 (see, e.g., Ji Q, et al. Engineered zinc-finger transcription factors activate OCT4 (POU5F1), SOX2, KLF4, c-MYC (MYC) and miR302/367. Nucleic Acids Res. 2014; 42:6158-6167; Perez-Pinera P, et al. Synergistic and tunable human gene activation by combinations of synthetic transcription factors. Nat Methods. 2013; 10:239-242; Farzadfard F, Perli S D, Lu T K. Tunable and multifunctional eukaryotic transcription factors based on CRISPR/Cas. ACS Synth Biol. 2013; 2:604-613; Black J B, Adler A F, Wang H G, et al. Targeted Epigenetic Remodeling of Endogenous Loci by CRISPR/Cas9-Based Transcriptional Activators Directly Converts Fibroblasts to Neuronal Cells. Cell Stem Cell. 2016; 19(3):406-414; and Maeder M L, Linder S J, Cascio V M, Fu Y, Ho Q H, Joung J K. CRISPR RNA-guided activation of endogenous human genes. Nat Methods. 2013; 10(10):977-979), p65 (see, e.g., Liu P Q, et al. Regulation of an endogenous locus using a panel of designed zinc finger proteins targeted to accessible chromatin regions. Activation of vascular endothelial growth factor A. J Biol Chem. 2001; 276:11323-11334; and Konermann S, et al. Genome-scale transcriptional activation by an engineered CRISPR-Cas9 complex. Nature. 2015; 517:583-588), HSF1, and RTA (see, e.g., Chavez A, et al. Highly efficient Cas9-mediated transcriptional programming. Nat Methods. 2015; 12:326-328). Example epigenetic modification domains can be obtained from, but are not limited to transcription repressors, such as, KRAB (see, e.g., Beerli R R, Segal D J, Dreier B, Barbas C F., 3rd Toward controlling gene expression at will: specific regulation of the erbB-2/HER-2 promoter by using polydactyl zinc finger proteins constructed from modular building blocks. Proc Natl Acad Sci USA. 1998; 95:14628-14633; Cong L, Zhou R, Kuo Y C, Cunniff M, Zhang F. Comprehensive interrogation of natural TALE DNA-binding modules and transcriptional repressor domains. Nat Commun. 2012; 3:968; Gilbert L A, et al. CRISPR-mediated modular RNA-guided regulation of transcription in eukaryotes. Cell. 2013; 154:442-451; and Yeo N C, Chavez A, Lance-Byrne A, et al. An enhanced CRISPR repressor for targeted mammalian gene regulation. Nat Methods. 2018; 15(8):611-616).

In example embodiments, the epigenetic modification domain linked to a DNA binding domain recruits an epigenetic modification protein to a target sequence. In example embodiments, a transcriptional activator recruits an epigenetic modification protein to a target sequence. For example, VP64 can recruit DNA demethylation, increased H3K27ac and H3K4me. In example embodiments, a transcriptional repressor protein recruits an epigenetic modification protein to a target sequence. For example, KRAB can recruit increased H3K9me3 (see, e.g., Thakore P I, D'Ippolito A M, Song L, et al. Highly specific epigenome editing by CRISPR-Cas9 repressors for silencing of distal regulatory elements. Nat Methods. 2015; 12(12):1143-1149). In an example embodiment, methyl-binding proteins linked to a DNA binding domain, such as MBD1, MBD2, MBD3, and MeCP2 recruits an epigenetic modification protein to a target sequence. In an example embodiment, Mi2/NuRD, Sin3A, or Co-REST recruit HDACs to a target sequence.

In example embodiments, the epigenetic modification domain can be a eukaryotic or prokaryotic (e.g., bacteria or Archaea) protein. In example embodiments, the eukaryotic protein can be a mammalian, insect, plant, or yeast protein and is not limited to human proteins (e.g., a yeast, insect, plant chromatin modifying protein, such as yeast HATs, HDACs, methyltransferases, etc.

In one aspect of the disclosure, is provided a fusion protein (epigenetic modification polypeptide) comprising from N-terminus to C-terminus, an epigenetic modification domain, an XTEN linker, and a nuclease-deficient RNA-guided DNA endonuclease enzyme or a nuclease-deficient endonuclease enzyme.

In aspects, the epigenetic modification polypeptide further comprises a transcriptional activator. In aspects, the transcriptional activator is VP64, p65, RTA, or a combination of two or more thereof. In another aspect, the epigenetic modification polypeptide further comprises one or more nuclear localization sequences. In embodiments, the epigenetic modification polypeptide comprises the nuclease-deficient RNA-guided DNA endonuclease enzyme. In embodiments, the fusion protein comprises the nuclease-deficient DNA endonuclease enzyme.

In some embodiments, the functional domains associated with the adaptor protein or the CRISPR enzyme is a transcriptional activation domain comprising VP64, p65, MyoD1, HSF1, RTA or SET7/9. Other references herein to activation (or activator) domains in respect of those associated with the adaptor protein(s) include any known transcriptional activation domain and specifically VP64, p65, MyoD1, HSF1, RTA or SET7/9 (see, e.g., U.S. Pat. No. 11,001,829B2).

In certain embodiments, the present disclosure provides a fusion protein comprising from N-terminus to C-terminus, an RNA-binding sequence, an XTEN linker, and a transcriptional activator. In aspects, the transcriptional activator is VP64, p65, RTA, or a combination of two or more thereof. In aspects, the fusion protein further comprises a demethylation domain, a nuclease-deficient RNA-guided DNA endonuclease enzyme or a nuclease-deficient endonuclease enzyme, a nuclear localization sequence, or a combination of two or more thereof. In embodiments, the fusion protein comprises the nuclease-deficient RNA-guided DNA endonuclease enzyme. In embodiments, the fusion protein comprises the nuclease-deficient DNA endonuclease enzyme.

In certain embodiments, the present disclosure provides a method of activating a target nucleic acid sequence in a cell, the method comprising: (i) delivering a first polynucleotide encoding a epigenetic modification polypeptide described herein including embodiments thereof to a cell containing the silenced target nucleic acid; and (ii) delivering to the cell a second polynucleotide comprising: (a) a sgRNA or (b) a cr:tracrRNA; thereby reactivating the silenced target nucleic acid sequence in the cell. In aspects, the sgRNA comprises at least one MS2 stem loop. In aspects, the second polynucleotide comprises a transcriptional activator. In aspects, the second polynucleotide comprises two or more sgRNA.

### Zinc Finger Nucleases

In some embodiments, the polynucleotide is modified using a Zinc Finger nuclease or system thereof. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

ZFPs can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

### TALE Nucleases

In some embodiments, a TALE nuclease or TALE nuclease system can be used to modify a polynucleotide. In some embodiments, the methods provided herein use isolated, non-naturally occurring, recombinant or engineered DNA binding proteins that comprise TALE monomers or TALE monomers or half monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, “TALE monomers” or “monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X₁₋₁₁—(X₁₂X₁₃)—X₁₄₋₃₃ or ₃₄ or ₃₅, where the subscript indicates the amino acid position and X represents any amino acid. X₁₂X₁₃ indicate the RVDs. In some polypeptide monomers, the variable amino acid at position 13 is missing or absent and in such monomers, the RVD consists of a single amino acid. In such cases the RVD may be alternatively represented as X*, where X represents X₁₂ and (*) indicates that X₁₃ is absent. The DNA binding domain comprises several repeats of TALE monomers and this may be represented as (X₁₋₁₁—(X₁₂X₁₃)—X₁₄₋₃₃ or ₃₄ or ₃₅), where in an advantageous embodiment, z is at least 5 to 40. In a further advantageous embodiment, z is at least 10 to 26.

The TALE monomers can have a nucleotide binding affinity that is determined by the identity of the amino acids in its RVD. For example, polypeptide monomers with an RVD of NI can preferentially bind to adenine (A), monomers with an RVD of NG can preferentially bind to thymine (T), monomers with an RVD of HD can preferentially bind to cytosine (C) and monomers with an RVD of NN can preferentially bind to both adenine (A) and guanine (G). In some embodiments, monomers with an RVD of IG can preferentially bind to T. Thus, the number and order of the polypeptide monomer repeats in the nucleic acid binding domain of a TALE determines its nucleic acid target specificity. In some embodiments, monomers with an RVD of NS can recognize all four base pairs and can bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011).

The polypeptides used in methods of the disclosure can be isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In some embodiments, polypeptide monomers having RVDs RN, NN, NK, SN, NH, KN, HN, NQ, HH, RG, KH, RH and SS can preferentially bind to guanine. In some embodiments, polypeptide monomers having RVDs RN, NK, NQ, HH, KH, RH, SS and SN can preferentially bind to guanine and can thus allow the generation of TALE polypeptides with high binding specificity for guanine containing target nucleic acid sequences. In some embodiments, polypeptide monomers having RVDs HH, KH, NH, NK, NQ, RH, RN and SS can preferentially bind to guanine and thereby allow the generation of TALE polypeptides with high binding specificity for guanine containing target nucleic acid sequences. In some embodiments, the RVDs that have high binding specificity for guanine are RN, NH RH and KH. Furthermore, polypeptide monomers having an RVD of NV can preferentially bind to adenine and guanine. In some embodiments, monomers having RVDs of H*, HA, KA, N*, NA, NC, NS, RA, and S* bind to adenine, guanine, cytosine and thymine with comparable affinity.

The predetermined N-terminal to C-terminal order of the one or more polypeptide monomers of the nucleic acid or DNA binding domain determines the corresponding predetermined target nucleic acid sequence to which the polypeptides of the disclosure will bind. As used herein the monomers and at least one or more half monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases, this region may be referred to as repeat 0. In animal genomes, TALE binding sites do not necessarily have to begin with a thymine (T) and polypeptides of the disclosure may target DNA sequences that begin with T, A, G or C. The tandem repeat of TALE monomers always ends with a half-length repeat or a stretch of sequence that may share identity with only the first 20 amino acids of a repetitive full-length TALE monomer and this half repeat may be referred to as a half-monomer. Therefore, it follows that the length of the nucleic acid or DNA being targeted is equal to the number of full monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

An exemplary amino acid sequence of a N-terminal capping region is:

(SEQ ID NO: 18)
M D P I R S R T P S P A R E L L S G P Q P D G V Q
P T A D R G V S P P A G G P L D G L P A R R T M S
R T R L P S P P A P S P A F S A D S F S D L L R Q
F D P S L E N T S L F D S L P P F G A H H T E A A
T G E W D E V Q S G L R A A D A P P P T M R V A V
T A A R P P R A K P A P R R R A A Q P S D A S P A
A Q V D L R T L G Y S Q Q Q Q E K I K P K V R S T
V A Q H H E A L V G H G F T H A H I V A L S Q H P
A A L G T V A V K Y Q D M I A A L P E A T H E A I
V G V G K Q W S G A R A L E A L L T V A G E L R G
P P L Q L D T G Q L L K I A K R G G V T A V E A V
H A W R N A L T G A P L N

An exemplary amino acid sequence of a C-terminal capping region is:

(SEQ ID NO: 19)
R P A L E S I V A Q L S R P D P A L A A L T N D H
L V A L A C L G G R P A L D A V K K G L P H A P A
L I K R T N R R I P E R T S H R V A D H A Q V V R
V L G F F Q C H S H P A Q A F D D A M T Q F G M S
R H G L L Q L F R R V G V T E L E A R S G T L P P
A S Q R W D R I L Q A S G M K R A K P S P T S T Q
T P D Q A S L H A F A D S L E R D L D A P S P M H
E G D Q T R A S

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the disclosure.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full-length capping region, while fragments that include the C-terminal 20 amino acids retain greater than 50% of the efficacy of the full-length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies can be generated by any of a number of computer programs known in the art, which include but are not limited to BLAST or FASTA. Suitable computer programs for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In some embodiments described herein, the TALE polypeptides of the disclosure include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the disclosure may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4X domain or a Kruppel-associated box (KRAB) or fragments of the KRAB domain. In some embodiments, the effector domain is an enhancer of transcription (i.e., an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the disclosure may include any combination of the activities described herein.

### Meganucleases

In some embodiments, a meganuclease or system thereof can be used to modify a polynucleotide. Meganucleases, which are endodeoxyribonucleases characterized by a large recognition site (double-stranded DNA sequences of 12 to 40 base pairs). Exemplary methods for using meganucleases can be found in U.S. Pat. Nos. 8,163,514, 8,133,697, 8,021,867, 8,119,361, 8,119,381, 8,124,369, and 8,129,134, which are specifically incorporated herein by reference.

### ARCUS Based Editing

In one example embodiment, a target gene is modified with an ARCUS base editing system. Exemplary methods for using ARCUS can be found in U.S. Pat. No. 10,851,358, US Publication No. 2020-0239544, and WIPO Publication No. 2020/206231 which are incorporated herein by reference.

### RNAi and Antisense Oligonucleotides (ASO)

In certain embodiments, a target gene is targeted with RNAi or antisense oligonucleotides (ASO). As used herein, “gene silencing” or “gene silenced” in reference to an activity of an RNAi molecule, for example a siRNA or miRNA refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%. Additionally, inhibitory nucleic acid molecules such as RNAi and ASOs can be used in vivo (see, e.g., Yan Y, Liu X Y, Lu A, Wang XY, Jiang L X, Wang J C. Non-viral vectors for RNA delivery. J Control Release. 2022; 342:241-279).

As used herein, the term “RNAi” refers to any type of interfering RNA, including but not limited to, siRNAi, shRNAi, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein). The term “RNAi” can include both gene silencing RNAi molecules, and also RNAi effector molecules which activate the expression of a gene.

As used herein, a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full-length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g., about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA” are used interchangeably herein are endogenous RNAs, some of which are known to regulate the expression of protein-coding genes at the posttranscriptional level. Endogenous microRNAs are small RNAs naturally present in the genome that are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated herein by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 1 16:281-297), comprises a dsRNA molecule.

Antisense therapy is a form of treatment that uses antisense oligonucleotides (ASOs) to target messenger RNA (mRNA). ASOs are capable of altering mRNA expression through a variety of mechanisms, including ribonuclease H mediated decay of the pre-mRNA, direct steric blockage, and exon content modulation through splicing site binding on pre-mRNA (see, e.g., Crooke S T, Liang X H, Baker B F, Crooke R M. Antisense technology: A review. J Biol Chem. 2021; 296:100416. doi:10.1016/j.jbc.2021.100416). Antisense oligonucleotides (ASO) generally inhibit their target by binding target mRNA and sterically blocking expression by obstructing the ribosome. ASOs can also inhibit their target by binding target mRNA thus forming a DNA-RNA hybrid that can be a substance for RNase H. Commonly used antisense mechanisms to degrade target RNAs include RNase H1-dependent and RISC-dependent mechanisms. Preferred ASOs include Locked Nucleic Acid (LNA), Peptide Nucleic Acid (PNA), and morpholinos.

### Small Molecules

In certain embodiments, a target gene is targeted with a small molecule inhibitor. In certain embodiments, receptors are targeted with small molecules that block ligand binding. In certain embodiments, a target gene is targeted with a degrader molecule. The term “small molecule” refers to compounds, preferably organic compounds, with a size comparable to those organic molecules generally used in pharmaceuticals. The term excludes biological macromolecules (e.g., proteins, peptides, nucleic acids, etc.). Preferred small organic molecules range in size up to about 5000 Da, e.g., up to about 4000, preferably up to 3000 Da, more preferably up to 2000 Da, even more preferably up to about 1000 Da, e.g., up to about 900, 800, 700, 600 or up to about 500 Da. In certain embodiments, the small molecule may act as an antagonist or agonist (e.g., blocking an enzyme active site or activating a receptor by binding to a ligand binding site).

### Small Molecule Degraders

In example embodiments, the one or more target genes specific to mesenchymal sensitivity is targeted with a small molecule degrader. One type of small molecule applicable to the present disclosure is a degrader molecule (see, e.g., Ding, et al., Emerging New Concepts of Degrader Technologies, Trends Pharmacol Sci. 2020 July; 41(7):464-474). The terms “degrader” and “degrader molecule” refer to all compounds capable of specifically targeting a protein for degradation (e.g., ATTEC, AUTAC, LYTAC, or PROTAC, reviewed in Ding, et al. 2020). Proteolysis Targeting Chimera (PROTAC) technology is a rapidly emerging alternative therapeutic strategy with the potential to address many of the challenges currently faced in modern drug development programs. PROTAC technology employs small molecules that recruit target proteins for ubiquitination and removal by the proteasome (see, e.g., Zhou et al., Discovery of a Small-Molecule Degrader of Bromodomain and Extra-Terminal (BET) Proteins with Picomolar Cellular Potencies and Capable of Achieving Tumor Regression. J. Med. Chem. 2018, 61, 462-481; Bondeson and Crews, Targeted Protein Degradation by Small Molecules, Annu Rev Pharmacol Toxicol. 2017 Jan. 6; 57: 107-123; and Lai et al., Modular PROTAC Design for the Degradation of Oncogenic BCR-ABL Angew Chem Int Ed Engl. 2016 Jan. 11; 55(2): 807-810). In certain embodiments, LYTACs are particularly advantageous for cell surface proteins. PROTACs can be synthesized for any target of interest, as evidenced by the hundreds of PROTACS available (see, e.g., Weng G, Cai X, Cao D, et al. PROTAC-DB 2.0: an updated database of PROTACs. Nucleic Acids Res. 2023; 51(D1):D1367-D1372). PROTACs have been demonstrated to be safe, efficacious, and to have clinical efficacy with meaningful benefits for patients (see, e.g., Bekes M, Langley D R, Crews C M. PROTAC targeted protein degraders: the past is prologue. Nat Rev Drug Discov. 2022; 21(3):181-200). PROTACs can be designed using fully synthetic, rationally designed small molecules. Id. In example embodiments, any druggable gene described herein can be targeted by rationale design starting with the drugs that bind to the gene products. In other example embodiments, the targeting molecule does not need to inhibit the gene product and small molecule libraries can easily be screened for molecules that bind to the target. Example target polypeptide binding moieties are also disclosed for example in, Sun et al., Signal Transduction and Targeted Therapy, 4:64 (2019), which provides exemplary proteins and corresponding ligands (i.e. target polypeptide binding moieties, see in particular FIGS. 5-48, which is incorporated herein by reference.

In example embodiments, the one or more agents capable of inhibiting the expression or activity of PTK2 is a PROTAC molecule (see, e.g., Cromm P M, Samarasinghe K T G, Hines J, Crews C M. Addressing Kinase-Independent Functions of Fak via PROTAC-Mediated Degradation. J Am Chem Soc. 2018; 140(49):17019-17026; Law R P, Nunes J, Chung C W, et al. Discovery and Characterisation of Highly Cooperative FAK-Degrading PROTACs. Angew Chem Int Ed Engl. 2021; 60(43):23327-23334; Gao H, Wu Y, Sun Y, Yang Y, Zhou G, Rao Y. Design, Synthesis, and Evaluation of Highly Potent FAK-Targeting PROTACs. ACS Med Chem Lett. 2019; 11(10):1855-1862; Gao H, Zheng C, Du J, et al. FAK-targeting PROTAC as a chemical tool for the investigation of non-enzymatic FAK function in mice. Protein Cell. 2020; 11(7):534-539; Popow J, Arnhof H, Bader G, et al. Highly Selective PTK2 Proteolysis Targeting Chimeras to Probe Focal Adhesion Kinase Scaffolding Functions. J Med Chem. 2019; 62(5):2508-2520; Ren C, Sun N, Liu H, et al. Discovery of a Brigatinib Degrader SIAIS164018 with Destroying Metastasis-Related Oncoproteins and a Reshuffling Kinome Profile. J Med Chem. 2021; 64(13):9152-9165; and Donovan K A, Ferguson F M, Bushman J W, et al. Mapping the Degradable Kinome Provides a Resource for Expedited Degrader Development. Cell. 2020; 183(6):1714-1731.e10).

In example embodiments, the one or more agents capable of inhibiting the expression or activity of MARK3 is a PROTAC molecule (see, e.g., Donovan K A, Ferguson F M, Bushman J W, et al. Mapping the Degradable Kinome Provides a Resource for Expedited Degrader Development. Cell. 2020; 183(6):1714-1731.e10).

In example embodiments, the one or more agents capable of inhibiting the expression or activity of CDC42 is a PROTAC molecule (see, e.g., Donovan K A, Ferguson F M, Bushman J W, et al. Mapping the Degradable Kinome Provides a Resource for Expedited Degrader Development. Cell. 2020; 183(6):1714-1731.e10).

### PHICS

In example embodiments, target proteins are targeted with chimeric molecules that recruit enzymes (e.g., kinases or phosphatases) to the target protein by a similar mechanism as PROTACs (see, e.g., Shoba V M, Munkanatta Godage D N P, Chaudhary S K, Deb A, Siriwardena S U, Choudhary A. Synthetic Reprogramming of Kinases Expands Cellular Activities of Proteins. Angew Chem Int Ed Engl. 2022; 61(29):e202202770; and International patent application publication No. WO 2021/142351A1). Phosphorylation-inducing chimeric small molecules (PHICS) can enable a kinase to act at a new cellular location or phosphorylate non-native substrates (neo-substrates)/sites (neo-phosphorylations). PHICS are formed by linking small-molecule binders of the kinase or the phosphatase and the target protein. The molecule that binds the target protein is the same as for PROTACs described herein and can be rationally designed in the same way. In example embodiments, modulating modifications at sites that regulate the target protein or at neo-sites inactivates or reduces the function of the target protein.

In example embodiments, CDS2 is a phosphoprotein and has post translation modifications at positions 21 (phosphoserine), 31 (phosphothreonine), 33 (phosphoserine), 35 (phosphoserine), 37 (phosphoserine), and 51 (phosphothreonine). In example embodiments, CDS2 is targeted with a kinase. In example embodiments, CDS2 is targeted with a phosphatase.

In example embodiments, ELMO2 is a phosphoprotein and has post translation modifications at positions 48 (phosphothreonine), 503 (phosphoserine), and 717 (phosphothreonine). In example embodiments, ELMO2 is targeted with a kinase. In example embodiments, ELMO2 is targeted with a phosphatase.

In example embodiments, ITGAV is a glycoprotein and has 13 N-Linked glycans modifications at positions 74, 290, 296, 488, 554, 615, 704, 835, 851, 874, 945, 973, and 980. In example embodiments, ITGAV is targeted with a glycosyltransferase. In example embodiments, ITGAV is targeted with a glycosylase.

### Methods for Detecting Target Genes or Metabolites

In example embodiments herein are methods of detecting correlated genes or metabolites in an EMT cancer (e.g., FIG. 3). In example embodiments herein are methods of detecting correlated genes or metabolites in an EMT cancer to determine tumors that have mesenchymal cell states that have increased dependency upon CDS2 (e.g., tumors more vulnerable to CDS2 inhibition) (e.g., FIGS. 5A-5C and FIGS. 6A-6C). In example embodiments, the metabolites or genes or gene products are referred to as biomarkers.

Biomarkers are useful in methods of monitoring, diagnosing, prognosing and/or staging treatment response in a subject by detecting a first level of expression, activity and/or function of one or more biomarker and comparing the detected level to a control of level wherein a difference in the detected level and the control level indicates that the presence of a treatment response in the subject. The terms “diagnosis” and “monitoring” are commonplace and well-understood in medical practice. By means of further explanation and without limitation the term “diagnosis” generally refers to the process or act of recognising, deciding on or concluding on a disease or condition in a subject on the basis of symptoms and signs and/or from results of various diagnostic procedures (such as, for example, from knowing the presence, absence and/or quantity of one or more biomarkers characteristic of the diagnosed disease or condition). The terms “prognosing” or “prognosis” generally refer to an anticipation on the progression of a disease or condition and the prospect (e.g., the probability, duration, and/or extent) of recovery. A good prognosis of the diseases or conditions taught herein may generally encompass anticipation of a satisfactory partial or complete recovery from the diseases or conditions, preferably within an acceptable time period. A good prognosis of such may more commonly encompass anticipation of not further worsening or aggravating of such, preferably within a given time period. A poor prognosis of the diseases or conditions as taught herein may generally encompass anticipation of a substandard recovery and/or unsatisfactorily slow recovery, or to substantially no recovery or even further worsening of such. The term “monitoring” generally refers to the follow-up of a disease or a condition in a subject for any changes which may occur over time. For example, changes in cancer subtype can be monitored during treatment, which can then direct treatment as described herein.

The biomarkers of the present disclosure are useful in methods of identifying patient populations at risk or suffering from an adverse tumor response based on a detected level of expression, activity and/or function of one or more biomarkers (e.g., resistance and/or elevated presence of cancer cells in a mesenchymal state). The biomarkers of the present disclosure are useful in methods of identifying patient populations that have a vulnerability, such as sensitivity to a drug or drug combination, including inhibitors of a target gene (e.g., CDS2). These biomarkers are also useful in monitoring subjects undergoing treatments and therapies for suitable or aberrant response(s) to determine efficaciousness of the treatment or therapy and for selecting or modifying therapies and treatments that would be efficacious in treating, delaying the progression of or otherwise ameliorating a symptom. The biomarkers provided herein are useful for selecting a group of patients at a specific state of a disease with accuracy that facilitates selection of treatments.

Distinct reference values may represent the prediction of a risk (e.g., an abnormally elevated risk) of having a given disease or condition as taught herein. In another example, distinct reference values may represent the diagnosis of such disease or condition of varying severity. In yet another example, distinct reference values may represent a good prognosis for a given disease or condition as taught herein vs. a poor prognosis for said disease or condition. In a further example, distinct reference values may represent varyingly favourable or unfavourable prognoses for such disease or condition. Reference values may be established according to known procedures previously employed for other cell populations, biomarkers and gene or gene product signatures. For example, a reference value may be established in an individual or a population of individuals characterised by a particular diagnosis, prediction and/or prognosis of said disease or condition (i.e., for whom said diagnosis, prediction and/or prognosis of the disease or condition holds true). Such population may comprise without limitation 2 or more, 10 or more, 100 or more, or even several hundred or more individuals.

A “deviation” of a first value from a second value may generally encompass any direction (e.g., increase: first value>second value; or decrease: first value<second value) and any extent of alteration.

For example, a deviation may encompass a decrease in a first value by, without limitation, at least about 10% (about 0.9-fold or less), or by at least about 20% (about 0.8-fold or less), or by at least about 30% (about 0.7-fold or less), or by at least about 40% (about 0.6-fold or less), or by at least about 50% (about 0.5-fold or less), or by at least about 60% (about 0.4-fold or less), or by at least about 70% (about 0.3-fold or less), or by at least about 80% (about 0.2-fold or less), or by at least about 90% (about 0.1-fold or less), relative to a second value with which a comparison is being made.

For example, a deviation may encompass an increase of a first value by, without limitation, at least about 10% (about 1.1-fold or more), or by at least about 20% (about 1.2-fold or more), or by at least about 30% (about 1.3-fold or more), or by at least about 40% (about 1.4-fold or more), or by at least about 50% (about 1.5-fold or more), or by at least about 60% (about 1.6-fold or more), or by at least about 70% (about 1.7-fold or more), or by at least about 80% (about 1.8-fold or more), or by at least about 90% (about 1.9-fold or more), or by at least about 100% (about 2-fold or more), or by at least about 150% (about 2.5-fold or more), or by at least about 200% (about 3-fold or more), or by at least about 500% (about 6-fold or more), or by at least about 700% (about 8-fold or more), or like, relative to a second value with which a comparison is being made.

Preferably, a deviation may refer to a statistically significant observed alteration. For example, a deviation may refer to an observed alteration which falls outside of error margins of reference values in a given population (as expressed, for example, by standard deviation or standard error, or by a predetermined multiple thereof, e.g., ±1×SD or ±2×SD or ±3×SD, or ±1×SE or ±2×SE or ±3×SE). Deviation may also refer to a value falling outside of a reference range defined by values in a given population (for example, outside of a range which comprises ≥40%, ≥50%, ≥60%, ≥70%, ≥75% or ≥80% or ≥85% or ≥90% or ≥95% or even ≥100% of values in said population).

In a further embodiment, a deviation may be concluded if an observed alteration is beyond a given threshold or cut-off. Such threshold or cut-off may be selected as generally known in the art to provide for a chosen sensitivity and/or specificity of the prediction methods, e.g., sensitivity and/or specificity of at least 50%, or at least 60%, or at least 70%, or at least 80%, or at least 85%, or at least 90%, or at least 95%.

For example, receiver-operating characteristic (ROC) curve analysis can be used to select an optimal cut-off value of the quantity of a given immune cell population, biomarker or gene or gene product signatures, for clinical use of the present diagnostic tests, based on acceptable sensitivity and specificity, or related performance measures which are well-known per se, such as positive predictive value (PPV), negative predictive value (NPV), positive likelihood ratio (LR+), negative likelihood ratio (LR−), Youden index, or similar.

In one embodiment, the biomarkers may be detected by immunoassays (described further herein), immunofluorescence (IF), immunohistochemistry (IHC), fluorescence activated cell sorting (FACS), mass spectrometry (MS), mass cytometry (CyTOF), any gene or transcript sequencing method, including but not limited to, RNA-seq, single cell RNA-seq, single nucleus RNA-seq, spatial transcriptomics, spatial proteomics, quantitative RT-PCR, single cell qPCR, FISH, RNA-FISH, MERFISH (multiplex (in situ) RNA FISH), Nanostring, in situ hybridization (ISH), CRISPR-effector system mediated screening assay (e.g. SHERLOCK assay), compressed sensing, and any combination thereof. Other methods including absorbance assays and colorimetric assays are known in the art and may be used herein. detection may comprise primers and/or probes or fluorescently bar-coded oligonucleotide probes for hybridization to RNA (see e.g., Geiss G K, et al., Direct multiplexed measurement of gene expression with color-coded probe pairs. Nat Biotechnol. 2008 March; 26(3):317-25). Other methods include microfluidics/nanotechnology sensors, and aptamer capture assay.

The present disclosure also may comprise a kit with a detection reagent that binds to one or more biomarkers or can be used to detect one or more biomarkers.

### Non-Invasive Detection

In preferred embodiments, the methods of detection are non-invasive. As used herein, non-invasive refers to a non-surgical procedure (i.e., no cutting or entering a body part using medical instruments). A blood test, taking a stool sample, or taking a sample with a swab is considered non-invasive herein. In example embodiments, biomarkers are measured in the blood, serum or plasma of the subject. In example embodiments, biomarkers are measured in the stool of the subject. In the context of the present disclosure, “blood” includes any blood fraction, for example serum, that can be analyzed according to the methods described herein. Serum is a standard blood fraction that can be tested. By measuring blood levels of a particular marker, it is meant that any appropriate blood fraction can be tested to determine blood levels and that data can be reported as a value present in that fraction. As a non-limiting example, the blood levels of a marker can be presented as pg/mL serum.

### Cell Free Transcripts

In an example embodiment, the biomarker is detected as a cell free transcript present in a blood sample (see, e.g., Larson M H, Pan W, Kim H J, et al. A comprehensive characterization of the cell-free transcriptome reveals tissue- and subtype-specific biomarkers for cancer detection. Nat Commun. 2021; 12(1):2357; Koh W, Pan W, Gawad C, et al. Noninvasive in vivo monitoring of tissue-specific global gene expression in humans. Proc Natl Acad Sci USA. 2014; 111(20):7361-7366; Qin J, Williams T L, Fernando M R. A novel blood collection device stabilizes cell-free RNA in blood during sample shipping and storage. BMC Res Notes. 2013; 6:380; and van Treijen M J C, Korse C M, van Leeuwaarde R S, et al. Blood Transcript Profiling for the Detection of Neuroendocrine Tumors: Results of a Large Independent Validation Study. Front Endocrinol (Lausanne). 2018; 9:740).

### Circulating Tumor Cells

In an example embodiment, the biomarker is detected in circulating tumor cells present in a blood sample. As used herein, “circulating tumor cell” or “CTC” refers to tumor cells which are shed from a tumor and present in the blood, i.e. in circulation. Cell markers (e.g. marker genes) can be used to identify and/or isolate CTCs from other components of the blood. Other example embodiments for isolating, capturing, or enriching a circulating tumor cell from a patient has been described (see, e.g., U.S. Pat. No. 10,900,083B2; US Patent application publication US20190107542A1; U.S. Pat. No. 10,022,659B2; and US Patent application publication US20210405055A1).

### Exosomes or Ribonucleoprotein (RNP) Complexes

In an example embodiment, the biomarker is detected in exosomes or ribonucleoprotein (RNP) complexes present in a blood sample. The terms “exosomes”, “micro-vesicles” and “extracellular vesicles” are herein used interchangeably. They refer to extracellular vesicles, which are generally of between 30 and 200 nm, for example in the range of 50-100 nm in size. In some aspects, the extracellular vesicles can be in the range of 20-300 nm in size, for example 30-250 nm in size, for example 50-200 nm in size. In some aspects, the extracellular vesicles are defined by a lipidic bilayer membrane. Common exosomal separation techniques include ultracentrifugation, density gradient centrifugation, dead-end filtration (DEF), tangential flow filtration (TFF), size-exclusion chromatography, and immunoaffinity (see, e.g., Chen J, Li P, Zhang T, et al. Review on Strategies and Technologies for Exosome Isolation and Purification. Front Bioeng Biotechnol. 2022; 9:811971). Exosomes may be purified as described (see, e.g., US Patent application publication US20180066307A1; and US Patent application publication US20190285618A1). RNPs (ribonucleoprotein particles) are complexes formed between RNA (including protein-coding mRNAs and non-protein-coding RNAs) and RNA-binding proteins (RBPs).

### Immunoassays

Immunoassay methods are based on the reaction of an antibody to its corresponding target or analyte and can detect the analyte in a sample depending on the specific assay format. To improve specificity and sensitivity of an assay method based on immunoreactivity, monoclonal antibodies are often used because of their specific epitope recognition. Polyclonal antibodies have also been successfully used in various immunoassays because of their increased affinity for the target as compared to monoclonal antibodies Immunoassays have been designed for use with a wide range of biological sample matrices. Immunoassay formats have been designed to provide qualitative, semi-quantitative, and quantitative results. In example embodiments, immunoassays can be used for non-invasive detection.

Quantitative results may be generated through the use of a standard curve created with known concentrations of the specific analyte to be detected. The response or signal from an unknown sample is plotted onto the standard curve, and a quantity or value corresponding to the target in the unknown sample is established.

Numerous immunoassay formats have been designed. ELISA or EIA can be quantitative for the detection of an analyte/biomarker. This method relies on attachment of a label to either the analyte or the antibody and the label component includes, either directly or indirectly, an enzyme. ELISA tests may be formatted for direct, indirect, competitive, or sandwich detection of the analyte. Other methods rely on labels such as, for example, radioisotopes (I¹²⁵) or fluorescence. Additional techniques include, for example, agglutination, nephelometry, turbidimetry, Western blot, immunoprecipitation, immunocytochemistry, immunohistochemistry, flow cytometry, Luminex assay, and others (see ImmunoAssay: A Practical Guide, edited by Brian Law, published by Taylor & Francis, Ltd., 2005 edition). Other advanced techniques, such as nonradioactive in situ hybridization (ISH), can be combined with immunochemistry to identify specific DNA or RNA molecules with fluorescent probes or tags that can be used for immunofluorescence and enzyme-linked fluorescence amplification.

Exemplary assay formats include enzyme-linked immunosorbent assay (ELISA), radioimmunoassay, fluorescent, chemiluminescence, and fluorescence resonance energy transfer (FRET) or time resolved-FRET (TR-FRET) immunoassays. Examples of procedures for detecting biomarkers include biomarker immunoprecipitation followed by quantitative methods that allow size and peptide level discrimination, such as gel electrophoresis, capillary electrophoresis, planar electrochromatography, and the like.

Exemplary assay formats also include ELISA and Luminex LabMAP immunoassays. The ELISA and Luminex LabMAP immunoassays are examples of sandwich assays. The term “sandwich assay” refers to an immunoassay where the antigen is sandwiched between two binding reagents, which are typically antibodies. The first binding reagent/antibody being attached to a surface and the second binding reagent/antibody comprising a detectable group. Examples of detectable groups include, for example and without limitation: fluorochromes, enzymes, epitopes for binding a second binding reagent (for example, when the second binding reagent/antibody is a mouse antibody, which is detected by a fluorescently-labeled anti-mouse antibody), for example an antigen or a member of a binding pair, such as biotin. The surface may be a planar surface, such as in the case of a typical grid-type array (for example, but without limitation, 96-well plates and planar microarrays), as described herein, or a non-planar surface, as with coated bead array technologies, where each “species” of bead is labeled with, for example, a fluorochrome (such as the Luminex technology described herein and in U.S. Pat. Nos. 6,599,331, 6,592,822 and 6,268,222), or quantum dot technology (for example, as described in U.S. Pat. No. 6,306,610).

In the bead-type immunoassays, such as the Luminex LabMAP system, the system incorporates polystyrene microspheres that are dyed internally with two spectrally distinct fluorochromes. Using precise ratios of these fluorochromes, an array is created consisting of 100 different microsphere sets with specific spectral addresses. Each microsphere set can possess a different reactant on its surface. Because microsphere sets can be distinguished by their spectral addresses, they can be combined, allowing up to 100 different analytes to be measured simultaneously in a single reaction vessel. A third fluorochrome coupled to a reporter molecule quantifies the biomolecular interaction that has occurred at the microsphere surface. Microspheres are interrogated individually in a rapidly flowing fluid stream as they pass by two separate lasers in the Luminex analyzer. High-speed digital signal processing classifies the microsphere based on its spectral address and quantifies the reaction on the surface in a few seconds per sample. The bead-type immunoassays are preferable for a number of reasons. As compared to ELISAs, costs and throughput are far superior. As compared to typical planar antibody microarray technology (for example, in the nature of the BD Clontech Antibody arrays, commercially available form BD Biosciences Clontech of Palo Alto, Calif.), the beads are far superior for quantitation purposes because the bead technology does not require pre-processing or titering of the plasma or serum sample, with its inherent difficulties in reproducibility, cost and technician time. For this reason, although other immunoassays, such as, without limitation, ELISA, RIA and antibody microarray technologies, are capable of use in the context of the present disclosure, but they are not preferred.

Methods of detecting and/or quantifying a detectable label or signal generating material depend on the nature of the label. The products of reactions catalyzed by appropriate enzymes (where the detectable label is an enzyme; see above) can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers.

Any of the methods for detection can be performed in any format that allows for any suitable preparation, processing, and analysis of the reactions. This can be, for example, in multi-well assay plates (e.g., 96 wells or 384 wells) or using any suitable array or microarray. Stock solutions for various agents can be made manually or robotically, and all subsequent pipetting, diluting, mixing, distribution, washing, incubating, sample readout, data collection and analysis can be done robotically using commercially available analysis software, robotics, and detection instrumentation capable of detecting a detectable label.

### Histology

Histology, also known as microscopic anatomy or microanatomy, is the branch of biology which studies the microscopic anatomy of biological tissues. Histology is the microscopic counterpart to gross anatomy, which looks at larger structures visible without a microscope. Although one may divide microscopic anatomy into organology, the study of organs, histology, the study of tissues, and cytology, the study of cells, modern usage places these topics under the field of histology. In medicine, histopathology is the branch of histology that includes the microscopic identification and study of diseased tissue. Biological tissue has little inherent contrast in either the light or electron microscope. Staining is employed to give both contrast to the tissue as well as highlighting particular features of interest. When the stain is used to target a specific chemical component of the tissue (and not the general structure), the term histochemistry is used. Antibodies can be used to specifically visualize proteins, carbohydrates, and lipids. This process is called immunohistochemistry, or when the stain is a fluorescent molecule, immunofluorescence. This technique has greatly increased the ability to identify categories of cells under a microscope. Other advanced techniques, such as nonradioactive in situ hybridization (ISH), can be combined with immunochemistry to identify specific DNA or RNA molecules with fluorescent probes or tags that can be used for immunofluorescence and enzyme-linked fluorescence amplification.

### Spatial Detection

In example embodiments, biomarkers are detected using a spatial detection method, in particular, for identifying subtypes in a tissue sample. An example spatial detection platform includes the digital spatial profiler (DSP), GeoMx DSP, which is built on Nanostring's digital molecular barcoding core technology and is further extended by linking the target complementary sequence probe to a unique DSP barcode through a UV cleavable linker (see, e.g., Li X, Wang C Y. From bulk, single-cell to spatial RNA sequencing. Int J Oral Sci. 2021; 13(1):36). A pool of such barcode-labeled probes is hybridized to mRNA targets that are released from fresh or FFPE tissue sections mounted on a glass slide. The slide is also stained using fluorescent markers (i.e., fluorescently conjugated antibodies) and imaged to establish tissue “geography” using the GeoMx DSP instrument. After the regions-of-interest (ROIs) are selected, the DSP barcodes are released via UV exposure and collected from the ROIs on the tissue. These barcodes are sequenced through standard NGS procedures. The identity and number of sequenced barcodes can be translated into specific mRNA molecules and their abundance, respectively, and then mapped to the tissue section based on their geographic location. The DSP barcode can also be linked to antibodies to detect proteins. An example spatial detection platform includes the CosMx Spatial Molecular Imager (Nanostring) platform, which enables high-plex (˜1,000 genes) spatial transcriptomics and proteomics at single cell and subcellular resolution (see, e.g., He, et al., High-plex Multiomic Analysis in FFPE at Subcellular Level by Spatial Molecular Imaging, bioRxiv 2021.11.03.467020). Other spatial detection methods or platform applicable to the present disclosure have been described (see, e.g., Li X, Wang C Y. From bulk, single-cell to spatial RNA sequencing. Int J Oral Sci. 2021; 13(1):36. Published 2021 Nov. 15. doi:10.1038/s41368-021-00146-0). The spatial data used in the present disclosure can be any spatial data. Methods of generating spatial data of varying resolution are known in the art, for example, ISS (Ke, R. et al. In situ sequencing for RNA analysis in preserved tissue and cells. Nat. Methods 10, 857-860 (2013)), MERFISH (Chen, K. H., Boettiger, A. N., Moffitt, J. R., Wang, S. & Zhuang, X. Spatially resolved, highly multiplexed RNA profiling in single cells. Science 348, (2015)), smFISH (Codeluppi, S. et al. Spatial organization of the somatosensory cortex revealed by cyclic smFISH. biorxiv.org/lookup/doi/10.1101/276097 (2018) doi:10.1101/276097), osmFISH (Codeluppi, S. et al. Spatial organization of the somatosensory cortex revealed by osmFISH. Nat. Methods 15, 932-935 (2018)), STARMap (Wang, X. et al. Three-dimensional intact-tissue sequencing of single-cell transcriptional states. Science 361, eaat5691 (2018)), Targeted ExSeq (Alon, S. et al. Expansion Sequencing: Spatially Precise In Situ Transcriptomics in Intact Biological Systems. biorxiv.org/lookup/doi/10.1101/2020.05.13.094268 (2020) doi:10.1101/2020.05.13.094268), seqFISH+ (Eng, C.-H. L. et al. Transcriptome-scale super-resolved imaging in tissues by RNA seqFISH+. Nature (2019) doi:10.1038/s41586-019-1049-y.), Spatial Transcriptomics methods (e.g., Spatial Transcriptomics (ST))(see, e.g., Stihl, P. L. et al. Visualization and analysis of gene expression in tissue sections by spatial transcriptomics. Science 353, 78-82 (2016))(now available commercially as Visium); Visium Spatial Capture Technology, 10× Genomics, Pleasanton, CA; WO2020047007A2; WO2020123317A2; WO2020047005A1; WO2020176788A1; and WO2020190509A9), Slide-seq (Rodriques, S. G. et al. Slide-seq: A scalable technology for measuring genome-wide expression at high spatial resolution. Science 363, 1463-1467 (2019)), or High Definition Spatial Transcriptomics (Vickovic, S. et al. High-definition spatial transcriptomics for in situ tissue profiling. Nat. Methods 16, 987-990 (2019)). In certain embodiments, proteomics and spatial patterning using antenna networks is used to spatially map a tissue specimen and this data can be further used to align single cell data to a larger tissue specimen (see, e.g., US20190285644A1). In certain embodiments, the spatial data can be immunohistochemistry data or immunofluorescence data.

### MS Methods

In example embodiments, nuclear magnetic resonance (NMR), liquid chromatography-mass spectrometry (LC-MS), or gas chromatography-mass spectrometry (GC-MS)] is used to determine metabolite abundance (see, e.g., Han J, Li Q, Chen Y, Yang Y. Recent Metabolomics Analysis in Tumor Metabolism Reprogramming. Front Mol Biosci. 2021; 8:763902). In example embodiments, the abundance of metabolites is determined by liquid chromatography/mass spectrometry (LC/MS) based metabolomics. In example embodiments, Fourier transform infrared (FT-IR) spectroscopy is used to detect metabolites (see, e.g., Xiao J F, Zhou B, Ressom H W. Metabolite identification and quantitation in LC-MS/MS-based metabolomics. Trends Analyt Chem. 2012; 32:1-14).

Gene detection may also be evaluated using mass spectrometry (MS) methods. A variety of configurations of mass spectrometers can be used to detect biomarker values. Several types of mass spectrometers are available or can be produced with various configurations. In general, a mass spectrometer has the following major components: a sample inlet, an ion source, a mass analyzer, a detector, a vacuum system, and instrument-control system, and a data system. Difference in the sample inlet, ion source, and mass analyzer generally define the type of instrument and its capabilities. For example, an inlet can be a capillary-column liquid chromatography source or can be a direct probe or stage such as used in matrix-assisted laser desorption. Common ion sources are, for example, electrospray, including nanospray and microspray or matrix-assisted laser desorption. Common mass analyzers include a quadrupole mass filter, ion trap mass analyzer and time-of-flight mass analyzer. Additional mass spectrometry methods are well known in the art (see Burlingame et al., Anal. Chem. 70:647 R-716R (1998); Kinter and Sherman, New York (2000)).

Protein biomarkers and biomarker values can be detected and measured by any of the following: electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), tandem time-of-flight (TOF/TOF) technology, called ultraflex III TOF/TOF, atmospheric pressure chemical ionization mass spectrometry (APCI-MS), APCI-MS/MS, APCI-(MS).sup.N, atmospheric pressure photoionization mass spectrometry (APPI-MS), APPI-MS/MS, and APPI-(MS).sup.N, quadrupole mass spectrometry, Fourier transform mass spectrometry (FTMS), quantitative mass spectrometry, and ion trap mass spectrometry.

Sample preparation strategies are used to label and enrich samples before mass spectroscopic characterization of protein biomarkers and determination biomarker values. Labeling methods include but are not limited to isobaric tag for relative and absolute quantitation (iTRAQ) and stable isotope labeling with amino acids in cell culture (SILAC). Capture reagents used to selectively enrich samples for candidate biomarker proteins prior to mass spectroscopic analysis include but are not limited to aptamers, antibodies, nucleic acid probes, chimeras, small molecules, an F(ab′)₂ fragment, a single chain antibody fragment, an Fv fragment, a single chain Fv fragment, a nucleic acid, a lectin, a ligand-binding receptor, affybodies, nanobodies, ankyrins, domain antibodies, alternative antibody scaffolds (e.g. diabodies etc.) imprinted polymers, avimers, peptidomimetics, peptoids, peptide nucleic acids, threose nucleic acid, a hormone receptor, a cytokine receptor, and synthetic receptors, and modifications and fragments of these.

### Single Cell Sequencing

In certain embodiments, the disclosure involves single cell RNA sequencing (see, e.g., Kalisky, T., Blainey, P. & Quake, S. R. Genomic Analysis at the Single-Cell Level. Annual review of genetics 45, 431-445, (2011); Kalisky, T. & Quake, S. R. Single-cell genomics. Nature Methods 8, 311-314 (2011); Islam, S. et al. Characterization of the single-cell transcriptional landscape by highly multiplex RNA-seq. Genome Research, (2011); Tang, F. et al. RNA-Seq analysis to capture the transcriptome landscape of a single cell. Nature Protocols 5, 516-535, (2010); Tang, F. et al. mRNA-Seq whole-transcriptome analysis of a single cell. Nature Methods 6, 377-382, (2009); Ramskold, D. et al. Full-length mRNA-Seq from single-cell levels of RNA and individual circulating tumor cells. Nature Biotechnology 30, 777-782, (2012); and Hashimshony, T., Wagner, F., Sher, N. & Yanai, I. CEL-Seq: Single-Cell RNA-Seq by Multiplexed Linear Amplification. Cell Reports, Cell Reports, Volume 2, Issue 3, p 666-673, 2012).

In certain embodiments, the disclosure involves plate based single cell RNA sequencing (see, e.g., Picelli, S. et al., 2014, “Full-length RNA-seq from single cells using Smart-seq2” Nature protocols 9, 171-181, doi:10.1038/nprot.2014.006).

In certain embodiments, the disclosure involves high-throughput single-cell RNA-seq. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. January; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Rosenberg et al., “Single-cell profiling of the developing mouse brain and spinal cord with split-pool barcoding” Science 15 Mar. 2018; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; Gierahn et al., “Seq-Well: portable, low-cost RNA sequencing of single cells at high throughput” Nature Methods 14, 395-398 (2017); and Hughes, et al., “Highly Efficient, Massively-Parallel Single-Cell RNA-Seq Reveals Cellular States and Molecular Features of Human Skin Pathology” bioRxiv 689273; doi: doi.org/10.1101/689273, all the contents and disclosure of each of which are herein incorporated by reference in their entirety.

In certain embodiments, the disclosure involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14(10):955-958; International Patent Application No. PCT/US2016/059239, published as WO2017164936 on Sep. 28, 2017; International Patent Application No. PCT/US2018/060860, published as WO/2019/094984 on May 16, 2019; International Patent Application No. PCT/US2019/055894, published as WO/2020/077236 on Apr. 16, 2020; Drokhlyansky, et al., “The enteric nervous system of the human and mouse colon at a single-cell resolution,” bioRxiv 746743; doi: doi.org/10.1101/746743; and Drokhlyansky E, Smillie C S, Van Wittenberghe N, et al. The Human and Mouse Enteric Nervous System at Single-Cell Resolution. Cell. 2020; 182(6):1606-1622.e23, which are herein incorporated by reference in their entirety.

### Hybridization Assays

Such applications are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of a signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively. Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the biomarkers whose expression is being assayed is contacted with target nucleic acids as described above. Contact is carried out under hybridization conditions, e.g., stringent hybridization conditions as described above, and unbound nucleic acid is then removed. The resultant pattern of hybridized nucleic acids provides information regarding expression for each of the biomarkers that have been probed, where the expression information is in terms of whether or not the gene is expressed and, typically, at what level, where the expression data, i.e., expression profile, may be both qualitative and quantitative.

Optimal hybridization conditions will depend on the length (e.g., oligomer vs. polynucleotide greater than 200 bases) and type (e.g., RNA, DNA, PNA) of labeled probe and immobilized polynucleotide or oligonucleotide. General parameters for specific (i.e., stringent) hybridization conditions for nucleic acids are described in Sambrook et al., supra, and in Ausubel et al., “Current Protocols in Molecular Biology”, Greene Publishing and Wiley-interscience, NY (1987), which is incorporated in its entirety for all purposes. When the cDNA microarrays are used, typical hybridization conditions are hybridization in 5×SSC plus 0.2% SDS at 65 C for 4 hours followed by washes at 25° C. in low stringency wash buffer (1×SSC plus 0.2% SDS) followed by 10 minutes at 25° C. in high stringency wash buffer (0.1SSC plus 0.2% SDS) (see Shena et al., Proc. Natl. Acad. Sci. USA, Vol. 93, p. 10614 (1996)). Useful hybridization conditions are also provided in, e.g., Tijessen, Hybridization With Nucleic Acid Probes”, Elsevier Science Publishers B.V. (1993) and Kricka, “Nonisotopic DNA Probe Techniques”, Academic Press, San Diego, Calif. (1992).

Further embodiments are illustrated in the following Examples which are given for illustrative purposes only and are not intended to limit the scope of the disclosure.

### EXAMPLES

### Example 1—Dependencies in Tumors that have Undergone an Epithelial-to-Mesenchymal Transition (EMT)

Applicants analyzed correlations between the composite EMT gene sets (Byers, L. A., et al., 2013. An Epithelial-Mesenchymal Transition Gene Signature Predicts Resistance to EGFR and PI3K Inhibitors and Identifies Axl as a Therapeutic Target for Overcoming EGFR Inhibitor Resistance. Clinical Cancer Research 19, 279-290; Groger, C. J., et al., 2012. Meta-Analysis of Gene Expression Signatures Defining the Epithelial to Mesenchymal Transition during Cancer Progression. PLoS ONE 7, e51136; and Taube, J. H., et al., 2010. Core epithelial-to-mesenchymal transition interactome gene-expression signature is associated with claudin-low and metaplastic breast cancer subtypes. Proc. Natl. Acad. Sci. U.S.A. 107, 15449-15454) and dependency scores (FIG. 1). Correlations between the composite EMT gene sets and dependency scores highlighted CDS2 as a top scoring positively correlated hit in epithelial cell lines. CDS2 was negatively correlated in mesenchymal cell lines. Thus, the mesenchymal cell state is dependent on CDS2.

Applicants analyzed correlations between the EMT signatures and drug sensitivities (FIG. 2). These correlations highlight YM-155 as the top positively correlated compound with the composite EMT signatures for the PRISM drug testing cohort. These correlations highlight niclosamide as the top positively correlated compound with the composite EMT signatures for the CTD² drug testing cohort.

Applicants analyzed the correlation of the RNA expression levels for genes and the EMT signatures (FIGS. 3A, 3B, and 3C). Higher expression of positively correlated genes in a subject indicates a higher EMT score, leading to treatment resistance in more mesenchymal subjects (i.e., EMT_UP). Positive correlations indicate increased RNA expression for that gene with an increasing EMT score, while negative correlations indicate decreased RNA expression for that gene with an increasing EMT score. Detection of the genes can provide early detection of treatment resistance. Applicants analyzed the correlation of metabolites and the EMT signatures (FIG. 3D). Positive correlations indicate a higher metabolite concentration with an increasing EMT score, while negative correlations indicate a lower metabolite concentration with an increasing EMT score. Higher metabolite concentration of positively correlated metabolites in a subject indicates a higher EMT score, leading to treatment resistance in more mesenchymal subjects (i.e., EMT_UP). Detection of the metabolite concentration can provide early detection of treatment resistance.

Applicants show a strong selective dependency on CDS2 in cell lines with low expression of CDS1 (FIG. 4). The dependency is seen more clearly with CRISPR targeting of CDS2 in cell lines as compared to RNAi.

Applicants analyzed all co-dependencies with CDS2 (FIG. 5A). Cell lines dependent on CDS2 are also dependent on a co-dependent gene that is positively correlated (lighter shade). The dependencies do not necessarily depend upon the expression level of the correlated and anti-correlated genes. Applicants analyzed CDS2 dependencies for which high/low RNA expression is correlated with decreased/increased dependency (lighter shade)(FIG. 5B). Thus, lower expression of positively correlated genes is correlated with dependency on CDS2. Low RNA expression of positively correlated genes is associated with an increase in CDS2 dependency. High RNA expression of positively correlated genes is associated with a decrease in CDS2 dependency. The correlation between CDS1 expression and CDS2 dependency is unidirectional (FIG. 5C). As CDS1 expression increases CDS2 dependency decreases, but as CDS2 expression increases there is no change in CDS1 dependency.

Applicants determined a correlation between CDS2 dependency and metabolite levels (FIG. 6A). Cells having a greater CDS2 dependency have an increase in the negatively correlated metabolic levels. Positive correlation of CDS2 RNA expression and the Cancer Cell Line Encyclopedia (CCLE) metabolite levels imply an increase in CDS2 expression is associated with an increase in metabolite levels.

FIG. 7 illustrates Epithelial-to-mesenchymal transition (EMT) and mesenchymal-to-Epithelial transition (MET). Markers for the epithelial state and mesenchymal state are shown, as well as key transcription factors required for the transition. Applicants have identified target genes and drug candidates that can target specific vulnerabilities in the mesenchymal state. Thus, the drug resistant cells can be eliminated and a patient can be effectively treated with a standard of care treatment. For example, treatment of a subject with niclosamide, YM-155, and/or an inhibitor of CDS2 to target the mesenchymal state can reduce or eliminate the population of drug-resistant mesenchymal state cancer cells that would otherwise be present in an untreated subject having the same cancer, thereby improving therapeutic outcomes for subjects also administered (either co-administered or in series/sequence) a standard of care treatment (as known in the art and described elsewhere herein).

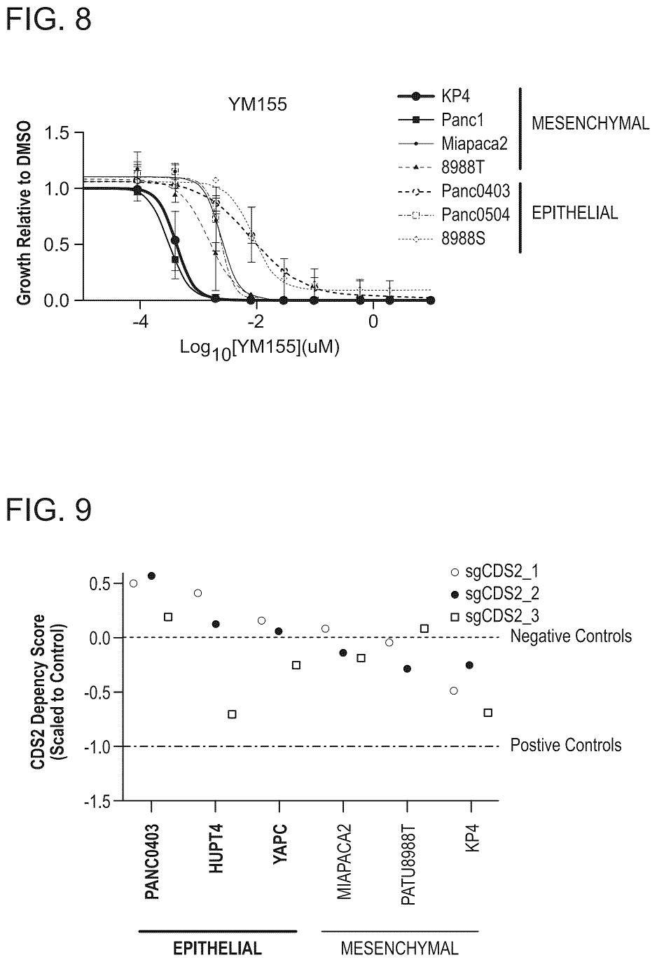

Validation studies were performed for two mesenchymal selective dependencies. In the first validation study, YM-155 was shown to be selective for inhibiting growth of mesenchymal cancer cells, as compared to epithelial cancer cells, with cell growth of each cancer cell line assessed using a cell-titer-glo assay (FIG. 8)

In the second validation study, three independent sgRNAs targeting CDS2 for knockdown (sgCDS2_1, sgCDS2_2, sgCDS2_3) in CRISPR-Cas experiments demonstrated that mesenchymal cells exhibited greater dependency on CDS2 than epithelial cells exhibited (FIG. 9).

### Example 2—Methods

Data Curation. All analyses were performed using publicly available data downloaded from the Dependency Map web portal, corresponding to the 22Q2 release. Drug testing data was from the 19Q4 release of PRISM and the most recent release (2015) of CTD (Gröger, C. J., et al., 2012). EMT gene sets were taken from the supplementals of the Byers (2013), Groger (2012), and Taube (2010) EMT related publications. Gene sets were broken down into up (mesenchymal) and down (epithelial) sub-gene sets. If up or down is not labeled in a figure, the composite gene set (union of the up and down lists) was used for scoring. Only carcinoma coded cell lines were used in analyses. These carcinoma lines were then subset further, when indicated, based on KRAS mutation or pancreas lineage.

Computational Methods. Analyses were performed using the statistical programming language R (v4.0.3, macOS 12.5.1, platform: x86_64-apple-darwin17.0 (64-bit)). EMT scores were generated using the GSVA package (v1.38.2) and Pearson correlations were calculated using the base cor function. All plots were generated using ggplot2 (v3.3.6). Data manipulation was performed using the dplyr (v1.0.8) and reshape2 (v.1.4.4) packages.

Various modifications and variations of the described methods, pharmaceutical compositions, and kits of the disclosure will be apparent to those skilled in the art without departing from the scope and spirit of the disclosure. Although the disclosure has been described in connection with specific embodiments, it will be understood that it is capable of further modifications and that the disclosure as claimed should not be unduly limited to such specific embodiments. Indeed, various modifications of the described modes for carrying out the disclosure that are obvious to those skilled in the art are intended to be within the scope of the disclosure. This application is intended to cover any variations, uses, or adaptations of the disclosure following, in general, the principles of the disclosure and including such departures from the present disclosure come within known customary practice within the art to which the disclosure pertains and may be applied to the essential features herein before set forth.

1-4. (canceled)

5. A method for treating a subject having a cancer capable of an epithelial-to-mesenchymal transition (an EMT cancer), the method comprising:
administering to the subject one or more agents capable of inhibiting the expression or activity of CDS2, ELMO2, ITGAV, AP1M1, FERMT2, CHMP4B, PTK2, ITGB5, PRKAR1A, ACTR1A, SRF, ZEB1, MTOR, DNM1L PSMB7, MOB4, TEAD1, DOCK5, ACTR3, BCAR1, UFM1, TIPARP, ILK, RSU1, WWTR1, NCKAP1, ATP6V1C1, MYH9, FOSL1, LIMS1, GNB1, EFR3A, ARHGEF7, WDR7, PKN2, CEMIP2, TEAD1, CTDNEP1, MARK3, VCL, CDC42, BCAR1, EGLN1, UFM1, GPX4, and/or CRK,
thereby treating the subject having the EMT cancer.

6-35. (canceled)

36. The method of claim 5, comprising administering to the subject one or more agents capable of inhibiting CDS2 expression or activity.

37. The method of claim 5, further comprising:
identifying the cancer as an EMT cancer by detecting in the cancer the expression of one or more genes identified as positively correlated with EMT Down signatures and/or as negatively correlated with EMT Up signatures,
wherein the one or more genes identified as positively correlated with EMT Down signatures are selected from among PTPN6, ILDR1, TSTD1, LCN2, MYO6, MFSD6, AMN, DDR1, PPL, C2orf15, AREG, CD9, FAM160A1, ENPP5, CD24, SMIM22, RAB25, PRRG2, CLDN4, RNF223, PLA2G4F, LLMO3, MAP7, MAL2, CBLC, KRTCAP3, MACC1, F11R, SPINT1, CNKSR1, CGN, IRF6, ESRP2, MPZL3, OVOL2, BSPRY, PRSS22, FXYD3, TJP2, PKP2, TMC5, RNF43, USP43, DLG3, PRKCZ, PKP3, ANO9, GRB7, JUP, CDH3, C11orf52, MYO5B, PATJ, TSPAN1, ZNF165, EPHA1, ELF3, BICDL22, TJP3, MUC20, MARVELD2, TC2N, CDS1, CLDN7, GRHL2, C1orf116, EPN3, PSD4, FAAH, OVOL1, SYNE4, IQANK1, MARVELD3, CRB3, KDF1, PRSS8, MAPK3, MAPK13, S100A14, ERBB3, GALNS, KRT19, EPS8L1, TMC4, ESRP1, SOWAHB, ST14, CDC42BPG, RAB17, CDH1, SH2D3A, LSR, B3GN3T, C1orf210, MPZL2, FUT3, SPINT2, C6orf132, PLEKHA7, TMC5, ICA1, TPD52L1, KLK8, SFTA2, KRT8, LAD1, IRF6, KLF5, PRR15, BICDL2, LIPH, LSR, FXYD3, EPCAM, MAL2, C2N, SPINT1, TCM5, ASS1, COBL, ACSL5, GPX2, MYZAP, KIAA1217, SCNN1A, B3GNT3, MISP, VGLL1, ITGB6, DOK7, HSH2D, SYT8, SH3YL1, SFN, TMEM125, TTC9, CST6, ACOT11, TUBA4A, PERP, ABHD11, BTC, CJB3, TNK1, LRRC1, ARRDC1, ANXA3, C11orf52, TCM4, ADIRF, LPAR5, GJB4, GJB5, MCTP2, TRIM31, HID1, DAPP1, SLPI, MST1R, LY75, CRYBG2, FUCA1, EPHA1, BIK, C1orf210, JUP, DDR1, LRG1, VAMP8, F3. CD9, TNK1, ILDR1, ITGB4, ESRP1, GALNT3, RNF223 MPZL2, EPCAM, B3GNT3, ST14, PTK7, GPR87, KCNK6, UPK2, HSH2D, KLF5, TMPRSS4, FRK, FAM83F, KRT19, TMC4, TC2N, PRRG2, RBM47, TC22, ITGB6, and TMEM125, and
wherein the one or more genes identified as negatively correlated with EMT Up signatures are selected from among PTCH2, TET3, AUNIP, E2F2, BEND3, CCSAP, INPP5J, RMND5B, PHF8, RAB11FIP4, OTUD3, KCNJ11, ZBTB39, STRBP, FZD3, SNK1, STRBP, PPFIA3, ADGRL1, FEM1A, MYCL, CHTOP, TICRR, CDS1, LHX4, PCNT, TCLD2, HDHD3, STRBP, CAMSAP3, C2orf15, DENND1C, IQANK1, MARVELD3, ZDHHC23, LNX2, ESRP2, BSPRY, LLGL2, SKY, CRB3, MAP7, MYH14, ERBB3, KDF1, OVOL1, HSD11B2, GLS2, MARVELD2, ARHGAP8, PRR5, CDS1, AP1Ar, EPB41, TET3, ESRP2, GAN, CDC42BPG, POU2F1, DDI2, ARHGAP8, STRBP, RREB1, LRRC8B, LLGL2, GRTP1, HOOK1, BSPRY, RDH13, SPATA2L, MCM9, PRR5, and RDH13.

38. The method of claim 5, further comprising:
identifying the cancer as an EMT cancer by detecting in the cancer or in the subject the presence of one or more of the following metabolites: stearoylcarnitine, C16.0.SM, betaine, palmitoylcarnitine, adenosine, 6.phosphogluconate, 1.methylnicotinamide, anthranilic.acid, carnosine, and/or sorbitol.

39. A method for identifying and treating a subject having a cancer capable of an epithelial-to-mesenchymal transition (an EMT cancer), the method comprising:
(a) detecting the expression in the cancer of one or more genes identified as positively correlated with CDS2 dependency selected from among PKP3, CDS1, MAL2, ELF3, IRF6, PATJ, CDH1, MAP7, ARHGEF16, ARHGEF5, TNK1, CNKSR1, ARHGAP8, CLDN4, IQANK1, CDH3, OVOL1, C11orf52, GRB7, GRHL2, C6orf132, MARVELD3, ESRP1, MPZL2, CHMP4C, TMEM125, TACSTD2, MYO5B, PRSS22, S100A14, SRP2, CRB3, EPCAM, RAB25, CLBC, TMEM184A, CLDN7, PRSS8, PRRG2, and SFN; and
(b) administering to the subject one or more agents capable of inhibiting CDS2 expression or activity,
thereby identifying and treating the subject having the EMT cancer.

40. The method of claim 39, wherein the one or more genes identified as positively correlated with CDS2 dependency is detected in the cancer below a reference value, wherein the reference value is determined by comparison to an appropriate control sample lacking the EMT cancer.

41. The method of claim 39, wherein the one or more genes comprises CDS1.

42. A method for identifying and treating a subject having a cancer capable of an epithelial-to-mesenchymal transition (an EMT cancer), the method comprising:
(a) detecting in the subject one or more metabolites identified as negatively correlated with CDS2 dependency, wherein the one or more metabolites is selected from among oxalate, C38.4.PC, AMP, C40.6.PC, C56.5.TAG, C50.0.TAG, trimethylamine.N.oxide, C58.7.TAG, C56.7.TAG, C36.4.PC.A, UMP, C22.1.SM, dCMP, C56.6.TAG, C58.6.TAG, C36.4.PC.B, C58.8.TAG, cytidine, CMP, C38.6.PC, alpha.glycophosphate, arachidonyl_carnitine, C38.5.PC, and C58.8.TAG; and
(b) administering to the subject one or more agents capable of inhibiting CDS2 expression or activity,
thereby identifying and treating the subject having the EMT cancer.

43. The method of claim 42, wherein the one or more metabolites identified as negatively correlated with CDS2 dependency is detected in the subject above a reference value, wherein the reference value is determined by comparison to an appropriate control subject or sample lacking the EMT cancer.

44. The method of claim 5, wherein the administering step is performed prior to, concurrently with, or after a primary cancer treatment.

45. The method of claim 5, further comprising:
detecting cancer cells that express a mesenchymal signature after the administering step and comparing to the number of cancer cells expressing a mesenchymal signature before the administering step, wherein the treatment is identified as efficacious in the subject if the number of tumor cells expressing a mesenchymal signature decreases.

46. The method of claim 5, wherein the one or more agents are one or more small molecules that reduce the activity or expression of CDS2 or the one or more genes.

47. The method of claim 46, wherein the one or more small molecules is capable of binding to the active site of CDS2, wherein the active site is an antagonistic analog of 1-stearoyl-2-arachidonoyl-sn-phosphatidic acid, or an anionic phospholipid end product or analog thereof, or phosphatidylinositol-(4,5)-bisphosphate or derivatives thereof.

48. The method of claim 46, wherein the one or more small molecules is a small molecule degrader capable of degrading CDS2 or the one or more genes.

49. The method of claim 5, wherein the one or more agents is an RNAi or an antisense oligonucleotide (ASO).

50. The method of claim 5, wherein the one or more agents is a transcriptional repressor system comprising a DNA binding element linked to or otherwise capable of complexing with a transcriptional repressor and configured to bind an enhancer of CDS2 or configured to bind the one or more genes.

51. The method of claim 5, wherein the one or more agents is an epigenetic modification polypeptide comprising a DNA binding element linked to or otherwise capable of associating with an epigenetic modification domain such that binding of the DNA binding element at a target sequence on gDNA results in one or more epigenetic modifications by the epigenetic modification domain that decreases expression of CDS2 or the one or more genes.

52. The method of claim 51, wherein the DNA binding element comprises a zinc finger protein or a DNA-binding domain thereof, a TALE protein or a DNA-binding domain thereof, or a Cas nuclease protein or a DNA-binding domain thereof.

53. The method of claim 5, wherein the one or more agents is a gene editing system configured to modify CDS2 or the one or more genes, an enhancer associated with CDS2 or the one or more genes, or a mRNA encoding CDS2 or the one or more genes, such that expression or activity of CDS2 or the one or more genes is reduced.

54. The method of claim 53, wherein the gene editing system is a zinc finger nuclease, a TALEN, a meganuclease, or a CRISPR-Cas system, a base editing system, CRISPR prime editing system, or a CRISPR-associated transposase (CAST) system.
